(19) United States
(12) Patent Application Publication
(10) Pub. No.: US 2006/0160848 A1
(43) Pub. Date: Jul. 20, 2006

(54) Method of controlling damage mediated by alpha, beta-unsaturated aldehydes

(76) Inventors: Philip C. Burcham, Valley View (AU); Frank R. Fontaine, Kenmore (AU); Simon M. Pyke, Bellevue Heights (AU); Lisa Kaminskas, Seaton (AU); Ian Musgrave, Largs North (AU)

Correspondence Address: BIRCH STEWART KOLASCH & BIRCH, PO BOX 747, FALLS CHURCH, VA, 22040-0747, US

(21) Appl. No.: 10/882,187

(22) Filed: Jul. 2, 2004

Related U.S. Application Data
(63) Continuation-in-part of application No. PCT/AU02/00900, filed on Jul. 5, 2002.

(30) Foreign Application Priority Data
Jan. 4, 2002 (AU) PR9813
Mar. 28, 2002 (AU) PS1415

(51) Int. Cl. A61K 31/47; A61K 31/15

(52) U.S. Cl. 514/310; 514/646; 514/664; 514/313

(57) ABSTRACT

This invention relates to a method for inhibiting the reaction of an α,β-unsaturated aldehyde with a biological molecule, the method including the step of administering hydralazine and/or dihydralazine in an amount that is effective to reduce the rate of reaction of the α,β-unsaturated aldehyde with the biological molecule.

### FIELD OF THE INVENTION

The present invention relates to methods of controlling damage in biological systems due to exposure to α,β-unsaturated aldehydes, and in particular to methods of reducing and/or reversing cell damage resulting from exposure to α,β-unsaturated aldehydes. The present invention also relates to methods for identifying molecules capable of reducing and/or reversing the damage to cells due to exposure to α,β-unsaturated aldehydes.

It will become apparent from the following description that the methods according to the present invention are most likely to relate to damage to biological systems due to exposure to acrolein. However, it must be appreciated that the invention is not to be limited in its application to damage to biological systems due to exposure to only acrolein.

### BACKGROUND OF THE INVENTION

Acrolein (I) is one of a number of α,β-unsaturated aldehydes that are known to be highly toxic and which are produced from a number of exogenous and endogenous sources. The medical significance of α,β-unsaturated aldehyde formation is likely to be considerable. In the case of acrolein, the molecule contributes to cell and tissue damage in individuals exposed to acrolein containing toxicants (eg smoke) and also in various diseases, conditions and states involving exposure to endogenous acrolein.

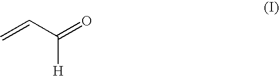

Acrolein is produced endogenously as a product of the peroxidation of unsaturated lipids, as well as during polyamine catabolism and the biotransformation of allyl compounds. Acrolein is also a pollutant produced during the combustion of biological matter, such as occurs during cigarette smoking, and the combustion of non-biological matter, such as occurs during combustion of plastics.

Acrolein is only one of a number of aldehydes that are produced during peroxidation of unsaturated lipids. Lipid peroxidation typically accompanies any condition involving overproduction (or impaired detoxification) of oxygen radicals, i.e. during oxidative stress. Other lipid-derived α,β-unsaturated aldehydes that are produced during oxidative stress include malondialdehyde, 4-hydroxydialkenals such as 4-hydroxynonenal, dienals, and a range of other 2-alkenals including crotonaldehyde. The chemical and toxicological properties of α,β-unsaturated aldehydes such as malondialdehyde and 4-hydroxynonenal have been studied most extensively. However, the role of acrolein is receiving increasing attention, as this molecule appears to be the most toxicologically significant aldehyde produced during lipid peroxidation.

Despite the knowledge of the gross toxicological properties of acrolein, the mechanism underlying its toxic effects is not well understood at a molecular level. Acrolein is toxic to a wide range of cell types and it is thought that this property arises at least in part because of the relative ease with which acrolein reacts with many of the biological molecules that are found in cells, including protein and DNA. Indeed, among all the α,β-unsaturated aldehydes produced in vivo, acrolein appears to be the strongest electrophile, and as such shows the highest reactivity with nucleophiles such as the sulfhydryl group of cysteine, the imidazole group of histidine and the amino group of lysine.

It appears that the α,β-unsaturated bond reacts rapidly with nucleophiles to form 1,4-addition adducts (Michael addition adducts), as shown in Scheme I for the reaction of acrolein with the amino group of a lysine residue.

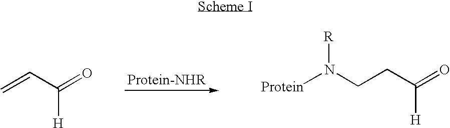

In the case of acrolein, it has been suggested that acrolein reacts with lysine residues proteins to form a number of intermediate products, such as mono- and bis-adducts, and that a cyclic adduct in which two molecules of acrolein are incorporated into the lysine side chain is eventually formed. The cyclic adduct formed (N^{α}-acetyl-N^{ε}-(3-formyl-3,4-dehydropiperidino)lysine) has been termed a FDP-lysine adduct and it has been postulated that it forms as shown in Scheme II.

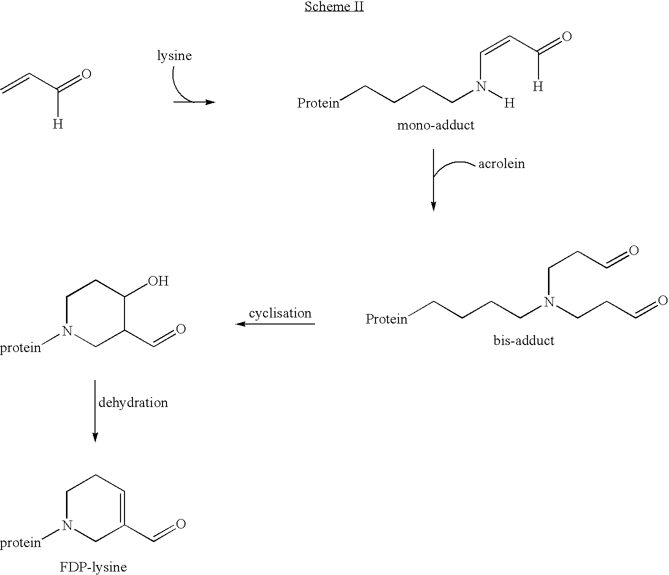

The toxicological significance of acrolein is likely to be due to the fact that acrolein shows a very pronounced ability to react with proteins. The products produced by the reaction of acrolein with proteins that cause toxicity are not well understood. The formation of FDP-lysine, or one or more of its precursors (for example mono- and bis-adducts), may be a major contributor to acrolein mediated toxicity.

Acrolein is well known to toxicology on account of its major contribution to the toxic properties of smoke and exhaust fumes. Acrolein is present in smoke produced upon combustion of a wide range of biological matter, including wood and tobacco, and upon combustion of non-biological matter including fossil fuels and plastics. Acrolein is also produced during photochemical oxidation of hydrocarbons in the atmosphere.

Although smoke contains a large number of noxious substances, the pathological effects of smoke exposure in victims are largely due to only a subset of the chemicals present. In particular, toxic aldehydes present within smoke are likely to contribute in large part to the pathological effects resulting from exposure to smoke. Indeed, animal data indicates that the presence of high levels of acrolein (10 to 250 ppm depending on the source of smoke and combustion conditions) in smoke plays a key role in the fatal lung injury seen in smoke exposure victims. Epithelial cells in the lung are highly vulnerable to damage by acrolein, and this can result in a breakdown of the integrity of the lung, leading to alveolar flooding and fatal pulmonary oedema. Serious irritation of the human lung results from exposure to air containing just 1 ppm acrolein. The Threshold Limit Value for safe workplace exposure to acrolein set by the American Conference of Governmental Industrial Hygienists (ACGIH) is just 0.1 ppm, among the lowest of all values for any compound.

Acrolein is also of considerable medical significance. Acrolein has a role in producing some of the serious side-effects that plague cancer patients receiving the anticancer drug, cyclophosphamide. Cyclophosphamide is used in the treatment of a diverse range of human tumours, including leukemias, lymphomas and multiple carcinomas (eg. breast, lung, ovary, cervix, etc). In addition, cyclophosphamide is used as an antiinflammatory agent in patients with advanced rheumatoid arthritis. It is also sometimes used as an immunosupressive in organ transplant recipients. The metabolic fate of cyclophosphamide in the body involves cytochrome P450-catalysed oxidation of the drug to a 4-hydroxy derivative, as shown in scheme III. The 4-hydroxy derivative undergoes a tautomerisation reaction to form aldophosphamide, an unstable intermediate that fragments to generate a nitrogen mustard derivative as well as acrolein. The acrolein so produced causes many of the toxic side-effects seen in chemotherapy patients receiving this drug. These include toxicity to the bladder (cystitis), and at higher doses, damage to the lungs, heart, liver and kidneys. Delayed toxic outcomes also occur in cyclophosphamide patients, such as leukemia, teratogenicity and sterility.

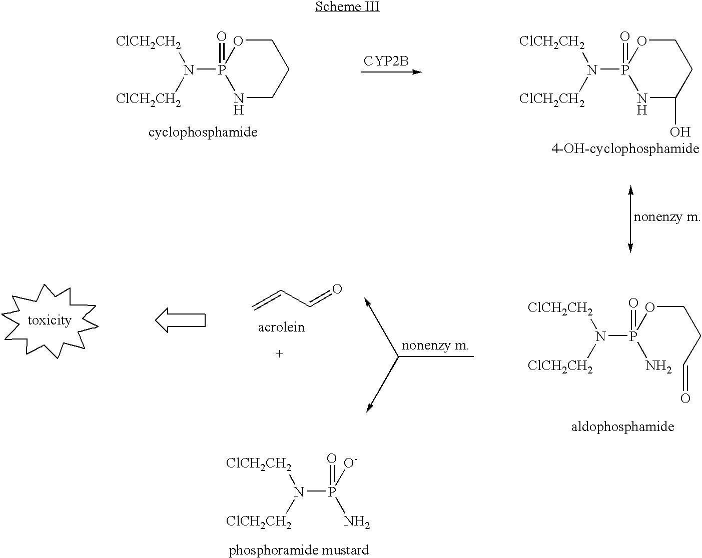

Acrolein has been identified as a significant mediator of cell and protein damage during oxidative damage to polyunsaturated fatty acids in cell membranes (lipid peroxidation). Since unsaturated lipids are very susceptible to damage by oxygen radicals, lipid peroxidation typically accompanies any cellular condition involving overproduction (or impaired detoxification) of oxygen radicals. Such a situation is termed “oxidative stress”. Although a number of reactive aldehydes form during lipid peroxidation, including malondialdehyde, 4-hydroxyalkenals such as 4-hydroxynonenal, dienals, and a range of other 2-alkenals, the pronounced electrophilicity of acrolein means that this molecule is among the most toxicologically-significant of these aldehydic products.

Because acrolein and other α,β-unsaturated aldehydes are formed as a by-product of oxidative membrane damage, it is likely that these molecules participate in any condition, state or disease in which oxidative stress features strongly. Evidence for an association of oxidative stress has been made in over 100 medical conditions. Oxidative stress is likely to play an especially significant role in chronic, degenerative diseases or conditions that accompany the ageing process. These include conditions such as neoplastic diseases, neurodegenerative diseases (eg. Alzheimer's, Parkinson's, Huntington's etc), CNS indications such as mild cognitive impairment and incipient dementia, vascular diseases (eg. atherosclerosis, stroke), diabetic complications (eg. nephropathy, retinopathy, vasculopathy etc), alcoholic liver disease, and ischemic tissue injury.

Indeed, acrolein has also been shown to contribute to cell and protein damage in a number of conditions and diseases including (i) acute or chronic smoke intoxication (ii) smoke-induced pulmonary oedema; (iii) atherosclerosis; (iv) Alzheimer's disease; (v) diabetic renal disease; (vi) dermal photodamage; and (vii) some forms of cell transformation and neoplasia. The participation of acrolein in these diseases and conditions may be either via exposure to exogenous acrolein sources, or via endogenous production via lipid peroxidation.

A major target for cell damage by chronic exposure to endogenously-produced acrolein is the CNS. Such acrolein production may contribute to the neuronal injury seen in chronic neurodegenerative diseases such as Alzheimer's disease and Parkinson's. A clear increase in extractable acrolein and protein-bound acrolein has been observed at sites of neuronal damage in the brains of Alzheimer's patients.

As discussed above, there are many situations in which α,β-unsaturated aldehydes such as acrolein are produced exogenously or endogenously and which may detrimentally affect biological systems by reacting with biomolecules (such as proteins) within the biological system. Accordingly, there is a need for reagents and/or methods that can be used to reduce the damage mediated by α,β-unsaturated aldehydes and as such reduce the effects of damage mediated by α,β-unsaturated aldehydes in a biological system. There is also a need to identify methods that may be used to screen new reagents that may be useful in reducing the damage mediated by α,β-unsaturated aldehyde in a biological system.

### SUMMARY OF THE INVENTION

The present invention provides a method for inhibiting the reaction of an α,β-unsaturated aldehyde with a biological molecule, the method including the step of administering hydralazine and/or dihydralazine in an amount that is effective to reduce the rate of reaction of the α,β-unsaturated aldehyde with the biological molecule.

The present invention also provides a method for reducing the damage mediated by an α,β-unsaturated aldehyde in a biological system, the method including the step of administering hydralazine and/or dihydralazine in an amount that is effective to reduce the damage mediated by the α,β-unsaturated aldehyde in the biological system.

The present invention further provides a method for reversing the damage mediated by α,β-unsaturated aldehyde in a biological system, the method including the step of administering hydralazine and/or dihydralazine in an amount that is effective to reduce the concentration of pre-existing adducts of the α,β-unsaturated aldehyde with a biological molecule.

The present invention also provides a method for treating a disease or condition associated with damage mediated by an α,β-unsaturated aldehyde in a subject, the method including the step of administering a pharmaceutically effective amount of hydralazine and/or dihydralazine.

The present invention also provides a method for determining the extent of damage mediated by an α,β-unsaturated aldehyde in a biological system, the method including the step of determining the concentration of an α,β-unsaturated aldehyde-modified protein in the biological system.

The present invention further provides a method for determining the extent of reversible damage mediated by an α,β-unsaturated aldehyde in a biological system, the method including the step of determining the concentration of an α,β-unsaturated aldehyde-modified protein that is reversibly modified in the biological system.

The present invention also provides a method for identifying a molecule capable of reducing the concentration of an acrolein-modified protein in a cell, the method including the steps of:
(a) exposing the cell to a test molecule;
(b) determining the ability of the test molecule to reduce the concentration of an acrolein-modified protein in the cell; and
(c) identifying the test molecule as a molecule capable of reducing the concentration of an acrolein-modified protein in the cell.

The present invention also provides a method for identifying a molecule capable of protecting a cell against toxicity due to acrolein exposure, the method including the steps of:
(a) exposing the cell to a toxic concentration of acrolein or an acrolein precursor;
(b) exposing the cell so treated to a test molecule;
(c) determining the ability of the test molecule to reduce toxicity due to exposure to acrolein or the acrolein precursor; and
(d) identifying the test molecule as a molecule capable of protecting the cell against toxicity due to exposure to acrolein.

The present invention also provides a method for identifying a molecule capable of reversing the formation of an acrolein-protein adduct, the method including the steps of:
(a) contacting a protein molecule with acrolein so as to allow the formation of an acrolein-protein adduct;
(b) contacting the acrolein-protein adduct with a test molecule;
(c) determining the ability of the test molecule to reverse the formation of the acrolein-protein adduct; and
(d) identifying the test molecule as a molecule capable of reversing the formation of the acrolein-protein adduct.

The present invention arises out of studies into scavenging agents that may react with α,β-unsaturated aldehydes and thereby prevent or minimise the reaction of α,β-unsaturated aldehydes with intracellular biological molecules. In particular, it has been found that hydralazine and dihydralazine are particularly effective at reducing and/or inhibiting the deleterious effects of acrolein-mediated damage in biological systems. Without being bound by theory, it appears that not only are hydralazine and dihydralazine capable of acting as efficient scavengers of α,β-unsaturated aldehydes such as acrolein, but these two compounds may also be able to reverse the deleterious effects of the damage mediated by α,β-unsaturated aldehydes, by reacting with pre-existing α,β-unsatutated aldehyde-modified molecules and thereby preventing the formation of deleterious by-products. This reaction of hydralazine and/or dihydralazine with the pre-existing α,β-unsaturated aldehyde-modified molecules may result in a molecule that is substantially similar to the starting molecule before modification, or alternatively, results in another molecule that is not deleterious to the biological system.

For example, it has been found that mouse liver cells that have not been exposed to exogenous acrolein still produce acrolein-modified lysine containing proteins. Acrolein production in this case is thought to be a result of endogenous lipid peroxidation and concomitant endogenous acrolein production. It has been found that treatment of these cells with hydralazine or dihydralazine results in a lowering of the concentration of acrolein modified proteins, indicating that hydralazine or dihydralazine is able to break down lysine-acrolein adducts after they had begun to form, thus effectively reversing the effects of acrolein damage in cells.

Accordingly, hydralazine and dihydralazine may be able to reduce the effects of the damage caused by acrolein by not only preventing further damage by acrolein, but also by reversing the effects of acrolein damage that has already occurred.

The mechanism(s) by which hydralazine and dihydralazine are able to break down acrolein-lysine adducts is not fully understood. However, it appears that hydralazine and dihydralazine are active at a relatively early stage of adduct formation, and most probably prior to formation of the relatively stable, cyclic FDP-lysine adduct. In fact, in vitro studies indicate that no effective reversal of acrolein-lysine adducts appears possible when treatment with hydralazine does not commence before 60 minutes after exposure of proteins to a high concentration of acrolein.

Various terms that will be used throughout the specification have meanings that will be well understood by a skilled addressee. However, for ease of reference, some of these terms will now be defined.

The term “biological molecule” as used throughout the specification is to be understood to mean any molecule present in a cell that has the capacity to chemically react with one or more α,β-unsaturated aldehyde molecules. The term includes proteins, DNA, peptides, polypeptides, amino acids, mRNA, rRNA and tRNA and other molecules containing a nucleophilic group capable of reacting with an α,β-unsaturated aldehyde. In this regard, a method of inhibiting the reaction of an α,β-unsaturated aldehyde with a biological molecule is to be understood to mean any method that results in a reduction in the rate of reaction of an α,β-unsaturated aldehyde with the biological molecule.

It will also be appreciated that a biological molecule modified by reaction with an α,β-unsaturated aldehyde may be referred to as an “α,β-unsaturated aldehyde-modified molecule” or an “α,β-unsaturated aldehyde-molecule adduct”. For example, a protein modified by reaction with acrolein may be referred to as an “acrolein-modified protein” or an “acrolein-protein adduct”. Additionally, the reaction of acrolein with a lysine residue in a protein may be referred to as an “acrolein-lysine adduct”.

The phrase “damage mediated by an α,β-unsaturated aldehyde” as used throughout the specification is to be understood to mean the reaction of an α,β-unsaturated aldehyde with one or more molecules present in a cell, the reaction directly or indirectly producing a chemical product that is in some way damaging to a cell, is deleterious to a cell or is toxic to a cell. The chemical product of the reaction may not necessarily be damaging, deleterious or toxic in itself, but may give rise to a further chemical product (by way of further reactions and/or metabolism of the first product) that is damaging, deleterious or toxic to a cell.

In this regard, a reduction in the damage mediated by an α,β-unsaturated aldehyde is to be understood to mean a reduction in the damage that occurs in a biological system as a result of the reaction of an α,β-unsaturated aldehyde with a biological molecule being inhibited and/or a reduction in the extent of pre-existing damage by the partial or complete reversal of the number of pre-existing adducts of biological molecules with the α,β-unsaturated aldehyde. As will be appreciated, a reduction in such damage will result in an alleviation of the effects that the damage has on the biological system.

For example, the rate of reaction of acrolein with a biological molecule may be reduced by a compound that traps acrolein. Alternatively, once acrolein has reacted with a biological molecule and formed a chemical product that is deleterious to the cell, a further reaction may occur with the compound that converts the pre-existing deleterious product into one that is not deleterious to the cell.

The term “biological system” as used throughout the specification is to be understood to mean any cellular or multi-cellular system, and includes isolated cells to whole organisms. For example, the biological system may be isolated mouse hepatocyte cells, rat neuronal cells, human lung epithelial cells, a tissue in an animal or human subject suffering the effects of either acute or chronic exposure to either exogenous or endogenous acrolein, or an entire animal or human subject suffering the effects of either acute or chronic exposure to either exogenous or endogenous acrolein.

The term “hydralazine” as used throughout the specification is to be understood to mean the following chemical compound or any derivatives of the following compound that are functionally equivalent to it in terms of their ability to react with a biological molecule:

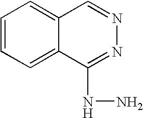

The term “dihydralazine” as used throughout the specification is to be understood to mean the following chemical compound or any derivatives of the following compound that are functionally equivalent to it in terms of their ability to react with a biological molecule:

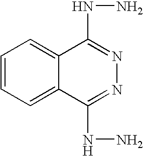

### BRIEF DESCRIPTION OF THE FIGURES

FIG. 1 shows the kinetics of acrolein-trapping by various amine compounds.

FIG. 2 shows the attenuation of allyl alcohol (AA) toxicity in mouse hepatocytes by hydralazine (HYD, Panel A) and dihydralazine (DIH, Panel B) from 5 to 50 μM.

FIG. 3 shows immunodetection of acrolein-modified lysine groups in proteins extracted from mouse hepatocytes after a 15 minutes exposure to acrolein alone or in combination with various concentrations of hydralazine or hydralazine alone.

FIG. 4 shows an immunoassay for “adduct-breaking” activity for various amine compounds. The assay substrate used was bovine serum albumin (BSA) that had been briefly pretreated with acrolein.

FIG. 5 shows the progressive loss of susceptibility of acrolein-lysine adducts to “adduct-breaking” actions of hydralazine with extended incubation at 37° C. The model protein BSA was treated with acrolein and incubated for up to 180 mins, incubated with hydralazine for 30 minutes, and aliquots removed and assessed for stability to hydralazine using immunoassay.

FIG. 6 shows that hydralazine displays concentration-dependent cytoprotective potency during both the “adduction” and “postadduction” phases of allyl alcohol toxicity in mouse hepatocytes.

FIG. 7 shows representative assay data obtained using the m-aminophenol assay for acrolein. Panel A shows a typical standard curve. Panel B shows the effect of spiking various dilutions of smoke extract with 20 nmol/mL acrolein.

FIG. 8 shows attenuation of LDH leakage in mouse hepatocytes exposed to 50 μM smoke-derived acrolein equivalents in the presence of hydralazine (HYD, Panel A) and dihydralazine (DIH, Panel B). Both drugs were added to give concentrations of 25, 50 and 100 μM.

FIG. 9 shows plasma sorbitol dehydrogenase (SDH) activities in the plasma 4 hours following the co-administration of allyl alcohol (AA, 100 mg/kg) and hydralazine (HYD, 0,100, 200 & 300 μmol/kg) to mice.

FIG. 10 shows the protection against cytotoxicity due to allylamine administration in rat neuronal cells by dihydralazine. Panel A shows the concentration-dependent decrease in viability of PC12 cells following a 24 hrs incubation in the presence of 2 to 200 μM allylamine. Panel B shows the protection against the cytotoxicity of 45 μM allylamine by PC12 cells after a 24 hr incubation in the presence of 0.1 to 100 μM dihydralazine.

FIG. 11 shows LDH leakage from isolated mouse hepatocytes after an 18-hr incubation in the presence of various concentrations of cyclophosphamide in Panel A (CPA, 0,100 to 2500 μM). Effect of proadifen (50 μM) on LDH leakage from isolated hepatocytes after an overnight incubation in the presence of 250 μM cyclophosphamide is shown in Panel B.

FIG. 12 shows the effect of various concentrations (10 to 100 μM) of hydralazine (Panel A) or dihydralazine (Panel B) on LDH leakage from isolated mouse hepatocytes after an 18-hr incubation in the presence of cyclophosphamide (CPA, 250 μM).

FIG. 13 shows loss of acrolein-lysine adducts in mouse hepatocytes accompanies protection against acute acrolein toxicity by hydralazine. (A) Cells were exposed to 0.5 mM acrolein in the presence and absence of 0.3 to 3 mM hydralazine, with aliquots of culture media removed for LDH determination at the times shown. Each data point represents the mean±S.E. of 3 independent observations. The various treatments are: controls, ⋄; 3 mM hydralazine, ♦; 0.5 mM acrolein, ▴; acrolein+0.3 mM hydralazine, ●; acrolein+1.0 mM hydralazine, ▪; acrolein+3.0 mM hydralazine, □. A (***) indicates significant difference between acrolein-treated cells and other treatments at the time point indicated (Bonferroni's post test, p<0.001). (B) Acrolein-lysine adducts were measured at 15 min, prior to overt loss of membrane integrity. The designations for the various lanes are: 1, control cells; 2, 3 mM hydralazine only; 3, 0.5 mM acrolein only; 4, 0.5 mM acrolein+0.3 mM hydralazine; 5, 0.5 mM acrolein+1.0 mM hydralazine; 6, 0.5 mM acrolein+3.0 mM hydralazine. The depicted blot is representative of results obtained in 2 independent experiments.

FIG. 14 shows electrospray ionization-mass spectrometry (ESI-MS) spectra obtained during analysis of acrolein- and hydralazine-modified preproenkephalin fragment 128 to 140 (PPE). (A) Effect of a 30 min reaction with acrolein, showing new ions due to formation of a Schiff base adduct (1), mono- (2) and bis-Michael (4) addition adducts, and the cyclised adduct FDP-lysine (3). (B) Addition of hydralazine generated several new ions due to hydrazone formation, namely (5) (formed from the mono-Michael adduct), (6) (derived from FDP-lysine) and (7) (derived from the bis-Michael adduct). Suggested structures for each species are shown in the bottom panel. The spectra shown are representative of results obtained during 5 independent replicates of the experiments.

FIG. 15 shows Immunochemical detection of hydralazine-trapped acrolein adducts in BSA. (A) Immunoreactivity of hydralazine/acrolein/KLH antiserum in a direct ELISA using either unmodified BSA (solid bars), acrolein-modified BSA (diagonal stripes), hydralazine-modified BSA (clear bars), or acrolein/hydralazine-modified BSA (horizontal stripes) as absorbed antigen. Acrolein/hydralazine-modified BSA was prepared by reacting BSA (2 mg/ml) with 5 mM acrolein (25 min) before 10 mM hydralazine was added for an additional 4 h. (B) Competitive ELISA using polyamino acid inhibitors to facilitate epitope characterization. The inhibitors were prepared as described in the Materials and Methods. The treatments were: unmodified polylysine (▾), unmodified polyhistidine, (♦), acrolein/hydralazine-modified polylysine (▪) and acrolein/hydralazine-modified polyhistidine (▴). Data are expressed as a percentage of control. In (A) and (B), the depicted data (mean±SE) were obtained during 2 independent experiments performed in quadruplicate. (C) Concentration-dependent adduct-trapping by hydralazine in acrolein-premodified BSA. BSA (10 mg/ml) was treated with 1 mM acrolein for 25 min at 37° C. in 50 mM sodium phosphate (pH 7.0) prior to the addition of hydralazine to give 0 (Lane 1), 50 (Lane 2), 100 (Lane 3), 250 (Lane 4) or 500 μM (Lane 5). After an additional 30 min reaction at 37° C., aliquots containing 20 μg BSA were resolved via SDS/PAGE and assessed via Western blot analysis.

FIG. 16 shows that adduct-trapping accompanies cytoprotection against acrolein-mediated toxicity by hydralazine. (A) Attenuation of LDH leakage during simultaneous exposure to allyl alcohol and hydralazine. (B) Attenuation of LDH leakage by hydralazine when present only during the “postadduction phase” of allyl alcohol toxicity. The treatments in (A) and (B) are: controls, ⋄; 100 μM allyl alcohol, ♦; 50 μM hydralazine, ▴; allyl alcohol+5 μM hydralazine, ●; allyl alcohol+10 μM hydralazine, ▪; allyl alcohol+25 μM hydralazine, Δ; allyl alcohol+50 μM hydralazine, ◯. In (A) and (B), each data point represents the mean±S.E. of 3 independent observations. In (A) and (B), differences between allyl alcohol only-treated cells and other treatments at various time points are indicated as follows (* p<0.05; ** p<0.01; *** p<0.001, Bonferroni's post test). In (C) and (D), cells were pretreated for 25 min with 100 μM allyl alcohol, then subsequently with hydralazine for 30 min. Next, cell lysates were prepared and 40 μg (C) or 60 μg (D) protein was resolved via SDS/PAGE (12.5% acrylamide gel). Western blot analysis was performed as described in the Materials and Methods. (C) The relevant lane designations are: 1: control—no allyl alcohol pretreatment; 2: no allyl alcohol pretreatment, 50 μM hydralazine in second phase; 3: allyl alcohol-pretreated only; 4: allyl alcohol-pretreated, then 5 μM hydralazine; 5: allyl alcohol-pretreated, then 10 μM hydralazine; 6: allyl alcohol-pretreated, then 25 μM hydralazine; 7: allyl alcohol-pretreated, then 50 μM hydralazine. (D) Detection of adduct-trapping at low hydralazine concentrations after loading 50% more protein per lane during SDS/PAGE. The lane contents are; 1: allyl alcohol-pretreated, then 2 μM hydralazine; 2: allyl alcohol-pretreated, then 4 μM hydralazine, 3: allyl alcohol-pretreated, then 6 μM hydralazine; 4: allyl alcohol-pretreated, then 8 μM hydralazine; 5: allyl alcohol-pretreated, then 10 μM hydralazine. The blots in (C) and (D) are representative of results obtained during 2 to 3 independent replicates of the experiment. (E) Results obtained during densitometric analysis of 3 hydralazine-labelled proteins highlighted in Panel D (see arrows).

FIG. 17 shows protection against allyl alcohol hepatotoxicity in mice. Hydralazine prevents elevations in plasma SDH (Panel A) and GPT (Panel B) activities but not hepatic GSH depletion (Panel C) in mice 4 hours after concurrent dosing with 90 mg/kg allyl alcohol (AA, i.p). Hydralazine (HYD; 100, 200 or 300 μmol/kg) was co-administered as a single i.p. dose with AA. Control mice received PBS, AA or 300 μmol/kg HYD (HYD300). Data are represented as mean±SEM of 6 to 8 animals per group. Data from treated animals was compared to controls via 1 way ANOVA followed by Dunn's (Panels A and B) or Dunnett's (Panel C) post-hoc tests. ** p<0.01, *** p<0.001 compared to vehicle control. †p<0.05, ††p<0.01 compared to M-treated mice.

FIG. 18 shows loss of hepatoprotection with delayed hydralazine administration. Mice received AA (90 mg/kg, i.p.) followed either immediately [co], 20 or 30 minutes later by hydralazine (HYD; 200 μmol/kg, i.p.). Four hours after the initial injection, animals were sacrificed for the determination of plasma SDH (Panel A) and liver GSH (Panel B). Values are reported as mean±SEM of 5 to 9 animals per group. SDH data from treated animals was compared to controls (PBS-treated) by 1-way ANOVA with a Dunn's post-hoc test whereas GSH data from treated mice was compared to control by a 1 way ANOVA with a Dunnett's post-hoc test. ** p<0.01, *** p<0.0001 compared to vehicle control.

FIG. 19 shows strong adduct-trapping accompanies hepatoprotection by hydralazine. Western blot showing dose-dependent adduct-trapping in liver proteins (125 μg/lane) of mice 60 minutes after concurrent administration of allyl alcohol (AA, 90 mg/kg) and hydralazine (HYD; 100-200 μmol/kg). Drug-trapped adducts were detected using rabbit antiserum raised against hydralazine/acrolein-modified KLH. The location of MW Markers was determined using Kaleidoscope prestained markers from BioRad (Hercules, CA). Lanes correspond to: (1) vehicle-treated, (2) AA-treated, (3) 100 μmol/kg HYD, (4) 200 μmol/kg HYD, (5 & 6) M plus 100 μmol/kg HYD and (7 & 8) AA plus 200 μmol/kg HYD. The arrows highlight two proteins (26 and 31 kDa) that were analyzed via densitometry.

FIG. 20 shows immunohistochemical detection of adduct-trapping in mouse liver. Images depict the distribution of hydralazine-stabilized, acrolein-adducted proteins in the right medial liver lobe of mice treated with AA and hydralazine. The various panels represent the following: Panel A (200× magnification)—liver section from a control, vehicle-treated animal. Panel B (200×)—liver section from a mouse 4 hours after it received 300 μmol/kg (1E)-acrylaldehyde 1-[1-phthalazinyl]-hydrazone. Panel C (200×)—section from mouse co-administered with M and 300 μmol/kg HYD; Panel D (400×) liver section from mouse co-administered AA and 100 μmol/kg HYD (N=nucleus, CM=cell membrane); Panels E and F (200×)—slices as per Panel C except the primary antibody was pre-incubated with 2 mg/mL hydralazine/acrolein-modified poly-L-lysine (Panel E) or hydralazine/acrolein-modified poly L-histidine (Panel F).

FIG. 21 shows that acrolein causes cross-linking of RNase A. Panel A shows the results of cross-linking studies using Coomassie Blue staining. Lane 1 is unmodified protein, lane 2 shows protein reacted with 0.75 mM acrolein, lane 3 shows protein reacted with 1.5 mM acrolein, lane 4 shows protein reacted with 3 mM protein, lane 5 shows protein reacted with 6 mM acrolein, lane 6 shows protein reacted with 12 mM acrolein, lane 7 shows methylated protein alone, lane 8 shows methylated protein reacted with 1.5 mM acrolein, and lane 9 shows 3 methylated protein reacted with 3 mM acrolein. Panel B shows Western analysis of an identical blot of acrolein treated Rnase A using rabbit antiserum selective for acrolein-modified lysine residues.

FIG. 22 shows the time course of lysine adduction and cross-linking by acrolein. RNase A was reacted with 3 mM acrolein over a time period of 4 hours. Panel A shows the results of the time course using Coomassie Blue staining. Lane 1 shows the reaction at 0 hours, lane 2 shows the reaction at 0.5 hours, lane 3 shows the reaction at 1.0 hour, lane 4 shows the reaction at 1.5 hours, lane 5 shows the reaction at 2.0 hours, lane 6 shows the reaction at 2.6 hours, lane 7 shows the reaction at 3.0 hours, lane 8 shows the reaction at 3.5 hours and lane 9 shows the reaction at 4.0 hours. Panel B shows Western analysis of an identical blot of acrolein treated Rnase A using rabbit antiserum selective for acrolein-modified lysine residues.

FIG. 23 shows that hydralzine inhibits cross-linking by trapping early adducts. RNase A was reacted with 3.2 mM acrolein. At 30 and 120 minutes after commencement of the reaction, aliquotes of the reaction were treated with hydralazine, to give a final concentration of 0.3, 1 or 3 mM hydralazine. Panel A shows the results using Coomassie Blue staining. Lane 1 shows unmodified RNase, lane 2 shows unmodified RNase and 3 mM hydralazine, lane 3 shows the reaction of acrolein modified RNAse after 30 minutes and treated with buffer, lane 4 shows the reaction of acrolein modified RNAse after 30 minutes and treated with 0.3 mM hydralazine, lane 5 shows the reaction of acrolein modified RNAse after 30 minutes and treated with 1 mM hydralazine, lane 6 shows the reaction of acrolein modified RNAse after 30 minutes and treated with 3 mM hydralazine, lane 7 shows the reaction of acrolein modified RNAse after 120 minutes and treated with buffer, lane 8 shows shows the reaction of acrolein modified RNAse after 120 minutes and treated with 0.3 mM hydralazine, lane 9 shows the reaction of acrolein modified RNAse after 120 minutes and treated with 1 mM hydralazine, and lane 10 shows the reaction of acrolein modified RNAse after 120 minutes and treated with 3 mM hydralazine. Panel B shows a similar gel after Western blotting with the antibody that detects acrolein-lysine modifications, and Panel C shows another gel after Western blotting with the antibody that detects hydralazine-trapped adducts.

FIG. 24 shows that hydralazine affords cytoprotection and induces adduct-trapping in PC-12 cells. Panel A shows the results of concurrent allylamine and hydralzine exposure. Panel B shows the results of a 4 hour delayed exposure of cells treated with allylamine to hydralazine. Panel C shows that a Western blot of cell proteins treated with hydralazine and allylamine using an antibody that detects hydralazine-trapped adducts. Lane 1 shows molecular weight markers, lane 2 shows PC12 cell proteins after treatment with 100 μM allylamine and 100 μM hydralazine, lane 3 shows PC12 cell proteins after treatment with 100 μM allylamine, lanes 4 and 5 are control lanes, lanes 6 and show PC12 cell proteins after treatment with 80 μM allylamine and 100 μM hydralazine, and lanes 8 and 9 show PC12 cell proteins after treatment with 80 μM allylamine.

FIG. 25 shows percent of remaining acrolein (Panel A) or crotonaldehyde (Panel B) in solution after reaction with equimolar scavengers at 37° C. in buffered solution. Data represented as mean±SEM, n=3.

FIG. 26 shows protection against allyl alcohol (AA, 100 μM) toxicity in isolated primary mouse hepatocytes by hydralazine (HYD, Panel A) and dihydralazine (DH, Panel B). AA and the protective hydrazines were co-incubated in RMPI medium for up to 3 hours. Aliquots of supernatant were taken at hourly intervals for assessment of LDH leakage from the cytoplasm into the culture medium. The concentration of hydrazine used is indicated in the legend as the number next to HYD or DH (μM). Data are represented as mean±SEM of the % of LDH leakage from the cytoplasm at each time point, n=3 observations.

FIG. 27 shows protection against crotyl alcohol (CA, 500 μM) toxicity in isolated primary mouse hepatocytes by hydralazine (HYD, Panel A) and dihydralazine (DH, Panel B). CA and the protective hydrazines were co-incubated in RMPI medium for up to 3 hours. Aliquots of supernatant were taken at hourly intervals for assessment of LDH leakage from the cytoplasm into the culture medium. The concentration of hydrazine used is indicated in the legend as the number next to HYD or DH (μM). Data are represented as mean±SEM of the % of LDH leakage from the cytoplasm at each time point, n=3 observations.

FIG. 28 shows hydralazine (HYD) mediated protection against hepatocellular toxicity induced by pentenal (Pent, 1 mM, Panel A), propargyl alcohol (PP, 1 mM, Panel B) and MDA (10 mM, Panel C). Hydralazine was co-incubated with the aldehydes and culture media from propargyl alcohol and pentenal treated cells taken at hourly intervals for 3 hours to assess lactate dehydrogenase leakage (% LDH) as an indicator of cell death. LDH was assessed at 2 hourly intervals from 12 to 18 hours for MDA. The concentrations of hydralazine added to the cells are indicated in the figure legends (in μM). Data are expressed as mean±SEM (3 replicates) of the % LDH leakage from the cytoplasm at each time point.

FIG. 29 shows scavenging of free acrolein from buffered solution at 37° C. by structurally diverse hydrazines (Panel A) and hydralazine analogues (Panel B). Acrolein (500 μM) was incubated with equimolar amounts of each of the hydrazine scavengers for up to 30 minutes and aliquots of the reaction mixture taken at 10 minute intervals for assessment of free acrolein by a HPLC assay. Data are represented as mean±SEM of remaining acrolein in solution, n=3 observations. Data for both hydralazine and dihydralazine is included in both panels to facilitate comparisons.

FIG. 30 shows protection against crotyl alcohol (CA, 500 μM) induced hepatocyte cell death afforded by 1-hydrazinoisoquinoline (HIQ, Panel A), 2-hydrazinoquinoline (HQL, Panel B), 4-hydrazinoquinazoline (HQZ, Panel C), 1,1-diphenylhydrazine (DPH, d) and benzylhydrazine (BH, e). Samples of culture media were taken 1, 2 and 3 hours after the co-addition of CA and the hydrazines (1-100 μM) for analysis of LDH leakage from the cytoplasm. The concentration of hydrazine used (μM) is indicated in the Figure legends. Data are represented as mean±SEM of the % lactate dehydrogenase leakage, n=3 observations.

### GENERAL DESCRIPTION OF THE INVENTION

As mentioned above, in one form the present invention provides a method of inhibiting the reaction of an α,β-unsaturated aldehyde with a biological molecule, the method including the step of administering hydralazine and/or dihydralazine in an amount that is effective to reduce the rate of reaction of the α,β-unsaturated aldehyde with the biological molecule.

The α,β-unsaturated aldehyde in the various forms of the present invention may be a substituted or non-substituted α,β-unsaturated aldehyde. Preferably, the α,β-unsaturated aldehyde is acrolein, malondialdehyde, 4-hydroxyalkenals including 4-hydroxynonenal, dienals, 2-alkenals, or the reactive α,β-unsaturated aldehyde tautomers of these compounds. Most preferably the α,β-unsaturated aldehyde is acrolein.

The biological molecule in the various forms of the invention may be any molecule present in a cell that has the capacity to chemically react with one or more α,β-unsaturated aldehyde molecules, including proteins, DNA, peptides, polypeptides, amino acids, mRNAs, rRNAs and tRNAs. Preferably, the biological molecule is a protein. More preferably, the biological molecule is a protein including one or more lysine residues, cysteine residues, or histidine residues, or a protein or polypeptide containing any combination of these residues. Most preferably, the biological molecule is a protein or polypeptide including one or more lysine residues.

The administration of hydralazine and/or dihydralazine in the various forms of the present invention may be within any time suitable to produce the desired effect. Preferably, administration occurs within 4 hours of exposure to an α,β-unsaturated aldehyde, more preferably within 2 hours, even more preferably within 1 hour, and most preferably within 30 minutes. It will be appreciated that the time periods referred to relate to the introduction of hydralazine or dihydralazine at the site of the damage mediated by an α,β-unsaturated aldehyde. The hydralazine or dihydralazine may be administered orally, parenterally, by inhalation or by any other suitable means and therefore transit time of the drug must be taken into account. Both hydralazine and dihydralazine have been used clinically as anti-hypertensive agents and therefore the pharmacological parameters of both compounds are understood.

The amount of hydralazine or dihydralazine used in the various forms of the present invention is not particularly limited, so long as it is within such an amount that generally exhibits a pharmacologically therapeutic effect. Preferably, the administration of hydralazine and/or dihydralazine to a subject is in the range from 0.1 to 100 μmol/kg. Most preferably, the administration of hydralazine and/or dihydralazine to a subject is in the range from 1 to 10 μmol/kg. The subject is preferably an animal or human subject.

The administration of hydralazine and dihyralazine in the various forms of the present invention may also include the use of one or more pharmaceutically acceptable additives, including pharmaceutically acceptable salts, amino acids, polypeptides, polymers, solvents, buffers, excipients and bulking agents.

In another form, the present invention provides a method for reducing damage mediated by an α,β-unsaturated aldehyde in a biological system, the method including the step of administering hydralazine and/or dihydralazine in an amount that is effective to reduce the damage mediated by the α,β-unsaturated aldehyde in the biological system.

The damage mediated by the α,β-unsaturated aldehyde in the biological system in the various forms of the present invention is damage that results from the formation of one or more adducts of a biological molecule with the α,β-unsaturated aldehyde. Preferably, the damage mediated by the α,β-unsaturated aldehyde is damage that results from the formation of one or more protein adducts with the α,β-unsaturated aldehyde in the biological system. More preferably, the damage mediated by the α,β-unsaturated aldehyde is damage that results from the formation of one or more protein adducts with acrolein in the biological system. Most preferably, the damage mediated by the α,β-unsaturated aldehyde is damage that results from the formation of one or more adducts of acrolein with one or more lysine residues of one or more proteins.

As will be appreciated, in the case of damage mediated by adducts of acrolein with lysine residues, to effect a reduction in damage the administration of hydralazine and/or dihydralazine should occur prior to substantial formation of FDP-lysine in the biological system.

The biological system in the various forms of the present invention may be any cellular or multi-cellular system and includes isolated cells to whole organisms. Preferably, the biological system is a cellular or multi-cellular system including cells derived from hepatocytes, neuronal cells, lung epithelial cells, cells undergoing oxidative stress, cells having been exposed to smoke, or cells associated with the following conditions, diseases or states, or cells associated with the onset of such conditions, diseases or states: chronic and/or degenerative diseases that accompany the ageing process (for example Alzheimer's, Parkinson's, Huntington's disease); CNS indications such as mild cognitive impairment or incipient dementia; neoplastic diseases; neurodegenerative diseases; vascular diseases (for example atherosclerosis, stroke); diabetic complications (for example nephropathy, retinopathy, vasculopathy); alcoholic liver disease; ischemic tissue injury; cells susceptible to injury during cyclophosphamide chemotherapy including bladder, ovary, breast, cervix and lung cells; cells susceptible to damage due to acute or chronic smoke inhalation, including gingivial cells; smoke-induced pulmonary oedema; atherosclerosis; diabetic renal disease; dermal photodamage; and cell transformation.

Preferably, the biological system is a multi-cellular system including cells susceptible to damage associated with the early stages of degenerative diseases or conditions that accompany the ageing process, such as Alzheimer's disease., or a multi-cellular system including cells associated with damage due to acute or chronic smoke exposure.

More preferably, the biological system is an animal or human subject suffering from a disease, condition or state that is associated with oxidative stress. More preferably, the biological system is an animal or human subject suffering from a disease, condition or state that is associated with either acute or chronic exposure to either exogenous or endogenous acrolein. More preferably, the biological system is an animal or human subject suffering from one or more of the following diseases or conditions: chronic and/or degenerative diseases that accompany the ageing process; neoplastic diseases; neurodegenerative diseases (for example Alzheimer's, Parkinson's, Huntington's disease); CNS indications such as mild cognitive impairment or incipient dementia; vascular diseases (for example stroke); diabetic complications (for example nephropathy, retinopathy, vasculopathy); alcoholic liver disease; ischemic tissue injury; cells susceptible to injury during cyclophosphamide chemotherapy including bladder, ovary, breast, cervix and lung cells; conditions due to acute or chronic smoke inhalation, including conditions involving gingivial cells; smoke-induced pulmonary oedema; atherosclerosis; diabetic renal disease; dermal photodamage; and cell transformation. Most preferably, the biological system is a human subject suffering from the acute or chronic effects of smoke inhalation or suffering from a disease, condition or state associated with the early stages of degenerative diseases or conditions that accompany the ageing process.

As discussed earlier, in a preferred form the present invention provides a method for alleviating the effects of damage mediated by acrolein in a biological system, the method including the step of administering hydralazine and/or dihydralazine in an amount that is effective to alleviate the effects of the damage mediated by acrolein. More preferably, the acrolein-mediated damage includes damage resulting from the formation of acrolein adducts to proteins.

Most preferably, the acrolein-mediated damage includes damage resulting from the formation of acrolein adducts to lysine residues of proteins. Preferably, the acrolein-mediated damage is associated with a disease, condition or state that is associated with oxidative stress.

As will be appreciated, this form of the invention may be used to reduce the damage mediated by acrolein in an animal or human subject suffering from the effects of acute or chronic damage mediated by acrolein. For example, this form of the invention may be used to reduce the damage mediated by the acute effects of smoke inhalation, such as cigarette smoking. Alternatively, this form of the invention may be used to reduce the damage mediated by acrolein in the early stages of neurodegenerative diseases such as Alzheimer's disease, to prevent the further progression of such diseases.

In a further preferred form, the present invention provides a method of reversing the damage mediated by an α,β-unsaturated aldehyde in a biological system, the method including the step of administering hydralazine and/or dihydralazine in an amount that is effective to reduce the concentration of pre-existing adducts of the α,β-unsaturated aldehyde with a biological molecule.

The biological molecule may be a protein, peptide, polypeptide, amino acid, DNA, mRNA, rRNA or tRNA. Preferably, the biological molecule is a protein. Most preferably, the biological molecule is a protein including a lysine residue.

As will be appreciated, this form of the present invention provides a method of reversing the effects of acrolein-mediated damage in a biological system. Preferably, the reversal of the effects of acrolein-mediated damage includes the administration of hydralazine and/or dihydralazine in an amount to reduce the concentration of pre-existing acrolein-protein adducts. More preferably, the reversal of the effects of acrolein-mediated damage includes administration of hydralazine and/or dihydralazine in an amount to reduce the concentration of pre-existing acrolein-lysine adducts. As will be further appreciated, in this form of the present invention the hydralazine and/or dihydralazine are administered so as to reach the site of acrolein damage prior to substantial formation of FDP-lysine adducts in the biological system.

This form of the present invention may be used to reverse the damage mediated by acrolein in a human subject suffering from the effects of acute or chronic damage mediated by acrolein. For example, these forms of the invention may be used to reverse the damage mediated by the acute effects of smoke inhalation, such as cigarette smoking. Alternatively, these forms of the invention may be used to reverse the damage mediated by acrolein in the early stages of neurodegenerative diseases such as Alzheimer's disease.

In a further preferred form, the present invention provides a method for treating a disease or condition associated with damage mediated by an α,β-unsaturated aldehyde in a subject, the method including the step of administering a pharmaceutically effective amount of hydralazine and/or dihydralazine.

This form of the present invention may also be used to reverse the damage mediated by acrolein in a human subject suffering from the effects of acute or chronic damage mediated by acrolein. For example, this form of the invention may also be used to reverse the damage mediated by acrolein in the early stages of neurodegenerative diseases such as Alzheimer's disease.

Preferably, the disease or condition associated with damage mediated by an α,β-unsaturated aldehyde is a disease or condition associated with oxidative stress. More preferably, the disease or condition associated with either acute or chronic exposure to either exogenous or endogenous acrolein. Most preferably, the disease or condition is one or more of the following diseases or conditions: chronic and/or degenerative diseases that accompany the ageing process; neoplastic diseases; neurodegenerative diseases (for example Alzheimer's, Parkinson's, Huntington's disease); CNS indications such as mild cognitive impairment or incipient dementia; vascular diseases (for example stroke); diabetic complications (for example nephropathy, retinopathy, vasculopathy); alcoholic liver disease; ischemic tissue injury; conditions associated with cyclophosphamide chemotherapy including cyclophosphamide chemotherapy of bladder, ovary, breast, cervix and lung cells; conditions due to acute or chronic smoke inhalation, including conditions involving gingivial cells; smoke-induced pulmonary oedema; atherosclerosis; diabetic renal disease; dermal photodamage; and diseases or conditions associated with cell transformation. Most preferably, the biological system is a subject suffering from the acute or chronic effects of smoke inhalation, or a subject suffering from a disease, condition or state associated with the early stages of degenerative diseases or conditions that accompany the ageing process.

For example, this form of the invention may be used to reverse the damage mediated by the acute effects of smoke inhalation in a subject, such as cigarette smoking. Preferably, the administration of hydralazine and/or dihydralazine occurs within 4 hours of exposure of the subject to smoke. More preferably, the administration of hydralazine and/or dihydralazine occurs within 2 hours of exposure of the subject to smoke. More preferably, the administration of hydralazine and/or dihydralazine occurs within 1 hour of exposure of the subject to smoke. Most preferably, the administration of hydralazine and/or dihydralazine occurs within 30 minutes of exposure of the subject to smoke.

The administration of hydralazine and/or dihydralazine to a subject is preferably in the range from 0.1 to 100 μmol/kg. Most preferably, the administration of hydralazine and/or dihydralazine to a subject is in the range from 1 to 10 μmol/kg. The subject is preferably an animal or human subject.

With regard to the administration of hydralazine and dihydralazine in the relevant forms of the present invention, hydralazine or dihydralazine can be prepared into a variety of pharmaceutical preparations in the form of, e.g., an aqueous solution, an oily preparation, a fatty emulsion, an emulsion, a gel, a dry powder etc., and these preparations can be administered as intramuscular or subcutaneous injection or as injection to the organ, or via an inhaler, or as an embedded preparation or as a transmucosal preparation through nasal cavity, rectum, uterus, vagina, lung, etc. The composition of the present invention can also be administered in the form of oral preparations (for example solid preparations such as tablets, capsules, granules or powders; liquid preparations such as syrup, emulsions or suspensions). Compositions containing hydralazine or dihydralazine may also contain a preservative, stabiliser, dispersing agent, pH controller or isotonic agent. Examples of suitable preservatives are glycerin, propylene glycol, phenol or benzyl alcohol. Examples of suitable stabilisers are dextran, gelatin, tocopherol acetate or alpha-thioglycerin. Examples of suitable dispersing agents include polyoxyethylene (20), sorbitan monoolelate (Tween 80), sorbitan sesquioleate (Span 30), polyoxyethylene (160) polyoxypropylene (30) glycol (Pluronic F68) or polyoxyethylene hydrogenated castor oil 60. Examples of suitable pH controllers include hydrochloric acid, sodium hydroxide and the like. Examples of suitable isotonic agents are glucose, D-sorbitol or D-mannitol.

A dose of hydralazine or dihydralazine according to the relevant forms of the present invention may be appropriately chosen, depending upon the amount of the composition containing the hydralazine or dihydralazine, kind of diseases or conditions to be treated, age and body weight of the patient, and frequency of administration.

The hydralazine or dihydralazine may be adminstered in the form of a composition containing a pharmaceutically acceptable carrier, diluent, excipient, suspending agent, lubricating agent, adjuvant, vehicle, delivery system, emulsifier, disintegrant, absorbent, preservative, surfactant, colorant, flavorant or sweetener.

For these purposes, the composition of the invention may be administered orally, parenterally, by inhalation spray, adsorption, absorption, topically, rectally, nasally, bucally, vaginally, intraventricularly, via an implanted reservoir in dosage formulations containing conventional non-toxic pharmaceutically-acceptable carriers, or by any other convenient dosage form. The term parenteral as used herein includes subcutaneous, intravenous, intramuscular, intraperitoneal, intrathecal, intraventricular, intrasternal, and intracranial injection or infusion techniques.

When administered parenterally, the composition will normally be in a unit dosage, sterile injectable form (solution, suspension or emulsion) which is preferably isotonic with the blood of the recipient with a pharmaceutically acceptable carrier. Examples of such sterile injectable forms are sterile injectable aqueous or oleaginous suspensions. These suspensions may be formulated according to techniques known in the art using suitable dispersing or wetting agents and suspending agents. The sterile injectable forms may also be sterile injectable solutions or suspensions in non-toxic parenterally-acceptable diluents or solvents, for example, as solutions in 1,3-butanediol. Among the acceptable vehicles and solvents that may be employed are water, saline, Ringer's solution, dextrose solution, isotonic sodium chloride solution, and Hanks' solution. In addition, sterile, fixed oils are conventionally employed as solvents or suspending mediums. For this purpose, any bland fixed oil may be employed including synthetic mono- or di-glycerides, corn, cottonseed, peanut, and sesame oil. Fatty acids such as ethyl oleate, isopropyl myristate, and oleic acid and its glyceride derivatives, including olive oil and castor oil, especially in their polyoxyethylated versions, are useful in the preparation of. injectables. These oil solutions or suspensions may also contain long-chain alcohol diluents or dispersants.

Sterile saline is a preferred carrier. The carrier may contain minor amounts of additives, such as substances that enhance solubility, isotonicity, and chemical stability, for example anti-oxidants, buffers and preservatives.

When administered orally, the composition will usually be formulated into unit dosage forms such as tablets, cachets, powder, granules, beads, chewable lozenges, capsules, liquids, aqueous suspensions or solutions, or similar dosage forms, using conventional equipment and techniques known in the art. Such formulations typically include a solid, semisolid, or liquid carrier. Exemplary carriers include lactose, dextrose, sucrose, sorbitol, mannitol, starches, gum acacia, calcium phosphate, mineral oil, cocoa butter, oil of theobroma, alginates, tragacanth, gelatin, syrup, methyl cellulose, polyoxyethylene sorbitan monolaurate, methyl hydroxybenzoate, propyl hydroxybenzoate, talc, magnesium stearate, and the like.

A tablet may be made by compressing or molding the active ingredient optionally with one or more accessory ingredients. Compressed tablets may be prepared by compressing, in a suitable machine, the active ingredient in a free-flowing form such as a powder or granules, optionally mixed with a binder, lubricant, inert diluent, surface active, or dispersing agent. Molded tablets may be made by molding in a suitable machine, a mixture of the powdered active ingredient and a suitable carrier moistened with an inert liquid diluent.

Compositions and methods of the invention also may utilize controlled release technology. The hydralazine or dihydralazine may also be administered as a sustained-release pharmaceutical. To further increase the sustained release effect, the composition may be formulated with additional components such as vegetable oil (for example soybean oil, sesame oil, camellia oil, castor oil, peanut oil, rape seed oil); middle fatty acid triglycerides; fatty acid esters such as ethyl oleate; polysiloxane derivatives; alternatively, water-soluble high molecular weight compounds such as hyaluronic acid or salts thereof (weight average molecular weight: ca. 80,000 to 2,000,000), carboxymethylcellulose sodium (weight average molecular weight: ca. 20,000 to 400,000), hydroxypropylcellulose (viscosity in 2% aqueous solution: 3 to 4,000 cps), atherocollagen (weight average molecular weight: ca. 300,000), polyethylene glycol (weight average molecular weight: ca. 400 to 20,000), polyethylene oxide (weight average molecular weight: ca. 100,000 to 9,000,000), hydroxypropylmethylcellulose (viscosity in 1% aqueous solution: 4 to 100,000 cSt), methylcellulose (viscosity in 2% aqueous solution: 15 to 8,000 cSt), polyvinyl alcohol (viscosity: 2 to 100 cSt), polyvinylpyrrolidone (weight average molecular weight: 25,000 to 1,200,000).

Alternatively, hydralazine and/or dihydralazine may be incorporated into a hydrophobic polymer matrix for controlled release over a period of days. The composition of the invention may then be molded into a solid implant, or externally applied patch, suitable for providing efficacious concentrations of hydralazine and/or dihydralazine over a prolonged period of time without the need for frequent re-dosing. Such controlled release films are well known to the art. Other examples of polymers commonly employed for this purpose that may be used include nondegradable ethylene-vinyl acetate copolymer a degradable lactic acid-glycolic acid copolymers which may be used externally or internally. Certain hydrogels such as poly(hydroxyethylmethacrylate) or poly(vinylalcohol) also may be useful, but for shorter release cycles than the other polymer release systems, such as those mentioned above.

The carrier may also be a solid biodegradable polymer or mixture of biodegradable polymers with appropriate time release characteristics and release kinetics. The composition may then be molded into a solid implant suitable for providing efficacious concentrations of hydralazine and/or dihydralazine over a prolonged period of time without the need for frequent re-dosing. The hydralazine or dihydralazine can be incorporated into the biodegradable polymer or polymer mixture in any suitable manner known to one of ordinary skill in the art and may form a homogeneous matrix with the biodegradable polymer, or may be encapsulated in some way within the polymer, or may be molded into a solid implant.

The present invention also provides a method for determining the extent of damage mediated by an α,β-unsaturated aldehyde in a biological system, the method including the step of determining the concentration of an α,β-unsaturated aldehyde-modified protein in the biological system. The diagnostic applications of such a method are readily apparent.

The detection of α,β-unsaturated aldehyde-modified proteins in the biological system in the various forms of the present invention may be by a suitable method known in the art, including detection utilising one or more suitable polyclonal or monoclonal antibodies raised to one or more α,β-unsaturated aldehyde-modified proteins. Detection of the modified proteins may be by a method that includes ELISA analysis, Western analysis, slot blot analysis or other methods of detecting proteins with the use of antibodies that are known in the art.

Alternatively, the detection of modified proteins may be by a method that includes mass spectrometry to detect adducts in the modified proteins.

In a preferred form, the present invention provides a method for determining the extent of acrolein-mediated damage in a biological system, the method including the step of determining the concentration of an acrolein-modified protein in the biological system.

Preferably, the acrolein-modified proteins include acrolein-lysine adducts. In one specific form, the acrolein-lysine adducts are FDP-lysine or one or more precursors to FDP-lysine.

The concentration of acrolein-modified products in the various forms of the present invention may be determined by a method known in the art. Preferably, the method of determining the concentration of acrolein-modified proteins includes the use of an antibody raised against an acrolein-modified protein to detect modified proteins. More preferably, the antibody is a polyclonal antibody. Most preferably, the antibody is a polyclonal antibody raised against acrolein-modified KLH (keyhole limpet hemocyanin).

The step of determining the concentration of one or more acrolein-modified proteins may be achieved by a suitable method known in the art, the method including the use of ELISA analysis, SDS/PAGE or slot blot analysis to detect adducts in the modified proteins. Alternatively, the concentration of the proteins may be determined by a method that includes mass spectrometry to detect adducts in the modified proteins.

As will be appreciated, the step of determining the concentration of one or more acrolein-modified proteins includes a step of lysing sufficient cells in the biological system to allow for detection of acrolein-modified cells. Preferably, the lysing of cells includes a step of lysing the cells in a buffer that does not include an amine buffer. More preferably, the lysing of cells includes a step of lysing the cells in a buffer that does not include a Tris buffer. In this regard, it has been determined that acrolein-modified proteins are unstable in amine buffers such as Tris buffers.

Preferably, the step of determining the concentration of one or more acrolein-modified proteins includes a step of preparing a cell lysate for analysis on a SDS/PAGE gel by preparing a buffer suitable for the loading of the cell lysate sample onto the SDS/PAGE gel. More preferably, the buffer suitable for loading does not contain reducing agents, including mercaptoethanol and/or dithiothreitol. In this regard, it has also been determined that acrolein-modified proteins are unstable in buffers containing reducing agents.

Preferably, the buffer suitable for loading is not heated prior to loading on a SDS/PAGE gel. In this regard, it has been further determined that acrolein-modified proteins present in a buffer suitable for analysis on SDS/PAGE gel are unstable to heating.

Preferably, the step of determining the concentration of one or more acrolein proteins includes a step of resolving proteins by SDS/PAGE and the use of a suitable antibody to detect the acrolein-modified proteins.

In another form, the present invention provides a method for determining the extent of reversible damage mediated by an α,β-unsaturated aldehyde in a biological system, the method including the step of determining the concentration of an α,β-unsaturated aldehydes-modified protein that is reversibly modified in the biological system.

The concentration of α,β-unsaturated aldehyde-modified proteins may be determined by a method known in the art, including the use of an antibody raised to an α,β-unsaturated aldehyde-modified protein or the use of mass spectrometry.

Preferably, the step of determining the concentration of an α,β-unsaturated aldehyde-modified protein that is reversibly modified may include the step of exposing the biological system with an amount of hydralazine and/or dihydralazine that is effective to reverse the damage in the biological system.

In a preferred form, the step of determining the concentration of acrolein-modified proteins that are reversibly modified may also include the step of exposing the biological system with an amount of hydralazine and/or dihydralazine that is effective to reverse the acrolein-mediated damage in the biological system. As will be appreciated, the concentration of reversibly modified proteins will be equal to the difference between the concentration of acrolein-modified products existing before treatment and the concentration of acrolein-modified products existing after treatment. The determination of the concentration of acrolein-modified proteins may be achieved as previously described, including the use of a method that utilises an antibody and/or mass spectrometry to detect adducts in the modified proteins.

Preferably, the step of determining the concentration of acrolein-modified proteins that are reversibly modified includes the step of detecting the concentration of proteins that include one or more FDP-lysine adducts, or one or more precursors to FDP-lysine. The concentration of reversibly modified proteins will be equal to the difference between the concentration of FDP-lysine products (or one or more precursors to FDP-lysine) existing before treatment and the concentration of FDP-lysine products (or one or more precursors to FDP-lysine) existing after treatment.

In another form, the present invention provides a method for identifying a molecule capable of reducing the concentration of an acrolein-modified protein in a cell, the method including the steps of:
(a) exposing the cell to a test molecule;
(b) determining the ability of the test molecule to reduce the concentration of an acrolein-modified protein in the cell; and
(c) identifying the test molecule as a molecule capable of reducing the concentration of an acrolein-modified protein in the cell.

As will be appreciated, molecules so identified may be tested for their ability to protect cells against the effects of acrolein-mediated damage. Accordingly, the present invention also provides molecules identified by such methods.

In one form, the acrolein-modified proteins are formed by the reaction of one or more proteins with endogenously produced acrolein. In another form, the cells are exposed to exogenous acrolein or an acrolein precursor.

The cells may be any suitable cells that allow for the determination of the concentration of one or more acrolein-modified proteins in the cell. Preferably, the cells are hepatocyte cells, neuronal cells or lung epithelial cells. More preferably, the cells are mouse hepatocyte cells, rat neuronal cells or human lung epithelial cells (for example human lung type II epithelial A549 cells). The cells may be primary cells or transformed cells. The cells may be cells cultured in vitro, or cells present in vivo in a whole animal or human.

In the case of endogenously produced acrolein, the endogenously produced acrolein is any acrolein present in a cell that is produced by one or more processes within the cell, including acrolein produced by lipid peroxidation. Accordingly, the cells must produce sufficient acrolein endogenously to allow determination of the ability of the test molecule to reduce the concentration of one or more acrolein-modified protein in the cell.

In the case of exposure of cells to exogenous acrolein, the cells will be any suitable type that allows exogenous acrolein to enter the cell and react with one or more proteins. Preferably, the cells for exposure to an acrolein precursor are hepatocyte, neuronal cells or lung epithelial cells. More preferably the cells are mouse hepatocyte cells, rat neuronal cells or human lung epithelial cells. The cells may be primary cells or transformed cells.

When the cell is to be exposed to an acrolein-precursor, the acrolein precursor is preferably allyl alcohol or allylamine. As will be appreciated, the choice of the acrolein precursor to be used will depend on the particular cell type to which the acrolein precursor is contacted. For example, in hepatocytes the acrolein precursor may be allyl alcohol. In neuronal cells, the acrolein precursor may be allylamine.

The cells for exposure to an acrolein precursor may be of any suitable type that allows the exogenously added precursor to enter the cells and be converted substantially into acrolein. Including cells engineered by recombinant means to allow the conversion of an acrolein precursor to acrolein. Preferably, the cells for exposure to an acrolein precursor are hepatocyte or neuronal cells. More preferably the cells are mouse hepatocyte cells or rat neuronal cells.

As will be appreciated, the ability of the test molecule to reduce the concentration of one or more acrolein-modified proteins will depend on the concentration of the test molecule. Accordingly, the concentration of the test molecule will be selected so as to determine the ability of the test molecule to reduce the concentration of one or more acrolein-modified proteins at the selected concentration. Preferably, the concentration of the test molecule will be less than 1 mM. More preferably the concentration of the test molecule will be less than 100 uM. Most preferably the concentration of the test molecule will be less than 10 uM. Exposure of the cells to the test molecule may be by a suitable method known in the art.

Determination of the of the ability of a test molecule to reduce the concentration of one or more acrolein-modified proteins in a cell may be by a suitable method known in the art, including the use of ELISA analysis, Western analysis or slot blot analysis with a suitable polyclonal or monoclonal antibody raised to one or more acrolein-modified proteins capable of detecting acrolein-modified proteins. Preferably, the antibody is a polyclonal antibody raised against acrolein-modified KLH.

Alternatively, the determination of the ability of a test molecule to reduce the concentration of one or more acrolein-modified proteins may be by a method that includes determination of the concentration of acrolien-modified proteins by mass spectrometry to detect adducts in the modified proteins.

Preferably, the acrolein-modified protein detected is a protein containing one or more acrolein-lysine adducts. In one specific form, the acrolein-lysine adduct is a FDP-lysine or one or more precursors to FDP-lysine.

As will be appreciated, the extent of reduction of the concentration of one or more acrolein-modified proteins will be equal to the difference between the concentration of acrolein-modified products existing before treatment and the concentration of acrolein-modified products existing after treatment with the test molecule.

The present invention also provides a method for identifying a molecule capable of protecting a cell against toxicity due to acrolein exposure, the method including the steps of:
(a) exposing the cell to a toxic concentration of acrolein or an acrolein precursor;
(b) exposing the cell so treated to a test molecule;
(c) determining the ability of the test molecule to reduce toxicity due to exposure to acrolein or the acrolein precursor; and
(d) identifying the test molecule as a molecule capable of protecting the cell against toxicity due to exposure to acrolein.

As will be appreciated, molecules so identified may be used to protect cells against the toxicity due to acrolein exposure. Accordingly, the present invention also provides molecules identified by such methods.

The cells may be any suitable cells that show toxicity due to acrolein exposure. Preferably, the cells are hepatocyte cells, neuronal cells or lung epithelial cells. More preferably, the cells are mouse hepatocyte cells, rat neuronal cells or human lung epithelial cells. The cells may be primary cells or transformed cells. The cells may be cells cultured in vitro or cells present in vivo in a whole animal or human.

The acrolein precursor is any molecule that may be taken up by a cell and converted substantially into acrolein.

The cells for exposure to acrolein may be of any suitable type that allows exogenous acrolein or the acrolein precursor to enter the cell and react with one or more proteins.

In this form of the present invention, the acrolein precursor may be taken up by a cell and converted substantially into acrolein. Toxicity may then occur as a result of acrolein-mediated damage to biological molecules in the cell. In this case, the cells may be any cells that allow the acrolein precursor to enter the cell and be converted substantially into acrolein, including cells engineered by recombinant means to allow the conversion of an acrolein precursor to acrolein.

Preferably, the acrolein -precursor is allyl alcohol or allylamine. As will be appreciated, the choice of the acrolein precursor to be used will depend on the particular cell type to which the acrolein precursor is contacted. For example, in mouse hepatocytes the acrolein precursor may be allyl alcohol. In neuronal cells, the acrolein precursor may be allylamine.

The toxicity of the acrolein precursor may be measured in a suitable manner that is known in the art and applicable to the cell type being tested. Toxicity will be understood in this context to mean any effect arising from uptake of exogenous acrolein, or the conversion of the acrolein precursor to acrolein, on the cell that is deleterious, damaging, or inhibitory. Cellular toxicity may be measured using a suitable method known in the art, including the use of probes for membrane integrity, cellular metabolic status or mitochondrial activity. Preferably, the toxicity is measured by the extent of leakage of a molecule from the treated cell or by the presence of an enzyme marker that is diagnostic of acrolein toxicity. More preferably, toxicity is measured by the extent of leakage of LDH from a cell or the activity of the enzyme sorbitol dehydrogenase.

As will be appreciated, a concentration of acrolein or the acrolein precursor must be selected that is toxic to the cells. Exposure of the cells to a toxic concentration of acrolein or the acrolein precursor may be by a suitable method known in the art, including the direct contact of acrolein or the acolein precursor with the cells, or alternatively, the expression of an acrolein precursor intracellularly.

As will be also appreciated, the ability of the test molecule to protect cells against toxicity will depend on the concentration of the test molecule. Accordingly, the concentration of the test molecule will be selected so as to determine the ability of the test molecule to provide protection at the selected concentration. Preferably, the concentration of the test molecule will be less than 1 mM. More preferably the concentration of the test molecule will be less than 100 uM. Most preferably the concentration of the test molecule will be less than 10 uM. Exposure of the cells to the test molecule may be by a suitable method well known in the art. The cells may be exposed to the test compound before, concurrently, or after the addition of acrolein or the acrolein precursor to the cells.

In a preferred form, the present invention provides a method for identifying a molecule capable of protecting a cell against the toxicity due to acrolein exposure by reversing the effect of acrolein-mediated damage to one or more biological molecules. Preferably, the biological molecule is a protein. More preferably, the biological molecule is a protein including one or more lysine residues.

In this form of the present invention, the test molecule will be added an amount of time after the addition of acrolein or the acrolein precursor sufficient to allow reversible modification to occur. In this regard, the cell is preferably exposed to the test molecule within 2 hours of exposure to the acrolein precursor. More preferably, the cell is exposed to the test molecule within 1 hour of exposure to the acrolein precursor. Most preferably, the cell is exposed to the test molecule within 30 minutes of exposure to the acrolein precursor.

In the case of toxicity due to the formation of acrolein adducts with lysine residues of one or more proteins, preferably the contacting of the test molecule occurs prior to the substantial formation of FDP-lysine after exposing the cell to a toxic concentration of acrolein or an acrolein precursor.

Confirmation of the ability of a test molecule to protect against toxicity due to reversible acrolein modification may be by the determination of the extent of reversible modification to one or more biological molecules in the cells exposed to the acrolein precursor. Confirmation of the reversible modification of a protein may be by a suitable method known in the art, including the determination of the extent of reversible modification by a method that includes the detection of modified biological molecules by mass spectrometry to detect adducts.

For example, detection of acrolein-modified proteins in the biological system may be by a suitable procedure, such as detection utilising one or more suitable polyclonal or monoclonal antibodies raised to one or more acrolein-modified proteins. Detection of the proteins may be by ELISA analysis, Western analysis, slot blot analysis or other methods of detecting proteins with the use of antibodies that are well known in the art. Preferably, the antibody is a polyclonal antibody raised against acrolein-modified KLH. Alternatively, the detection of modified proteins may be by a method that includes mass spectrometry to detect adducts in the modified proteins.

Preferably, the acrolein-modified protein detected for assessment of the reversible modification is a protein containing one or more acrolein-lysine adducts. In one specific form, the acrolein-lysine adduct is a FDP-lysine or one or more precursors to FDP-lysine.

As will appreciated, the extent of reversibly modified proteins will be equal to the difference between the concentration of acrolein-modified products existing before treatment and the concentration of acrolein-modified products existing after treatment.

Preferably, the step of determining the concentration of acrolein-modified proteins that are reversibly modified includes the step of detecting the concentration of proteins that include one or more FDP-lysine adducts, or detecting the concentration of one or more precursors to FDP-lysine. Once again, the concentration of reversibly modified proteins will be equal to the difference between the concentration of FDP-lysine products (or one or more precursors to FDP-lysine) existing before treatment and the concentration of FDP-lysine products (or one or more precursors to FDP-lysine) existing after treatment.

The present invention also provides a method for identifying a molecule capable of reversing the formation of an acrolein-protein adduct, the method including the steps of:
(a) contacting a protein molecule with acrolein so as to allow the formation of an acrolein-protein adduct;
(b) contacting the protein molecule so modified with a test molecule;
(c) determining the ability of the test molecule to reverse the formation of the acrolein-protein adduct; and
(d) identifying the test molecule as a molecule capable of reversing the formation of an acrolein-protein adduct.

In this context, a molecule capable of reversing the formation of an acrolein-protein adduct will be understood to mean a molecule capable of reacting with one or more of the acrolein-protein adducts and thereby (i) substantially regenerate the protein molecule as it existed before modification and/or (ii) prevent the formation of one or more acrolein-protein products that are deleterious to a cell. For example, the molecule may be capable of preventing the formation of FDP-lysine adducts with proteins.

As will be appreciated, molecules so identified may be used to reverse the formation of acrolein-protein adducts in cells and thereby reduce the damage mediated by acrolein exposure. Accordingly, the present invention also provides molecules identified by such methods.

The method according to this form of the invention may be performed either in an cell-free system or in cells. For example, the method may be performed in a suitable solution with one or more peptide, polypeptide or protein substrates and acrolein. An exemplary substrate is bovine serum albumin. Preferably, the substrate is substantially pure. As will be appreciated, the substrates will contain one or more groups capable of reacting with acrolein, such as lysine groups, histidine groups or cysteine groups.

Alternatively, the method according to this form of the invention may be performed in cells. Preferably the cells are animal or human cells. In this case the formation of acrolein-protein adducts may occur by the reaction of acrolein or an acrolein precursor with one or more proteins in the cell. The contacting of the protein with acrolein or an acrolein precursor may be by a suitable method known in the art, including the direct contact of the acolein precursor with the cells, or alternatively, the expression of the acrolein precursor intracellularly. The acrolein precursor is any molecule that may be taken up by a cell and converted substantially into acrolein. Preferably, the acrolein precursor is allyl alcohol or allylamine.

The cells may be cells in vitro in culture or in vivo cells in a whole organism. The cells are preferably isolated cells. More preferably, the cells are hepatocytes, neuronal cells. Most preferably, the cells are mouse hepatocytes, rat neuronal cells or lung epithelial cells.

As will be appreciated, if this form of the present invention is performed in cells, the choice of the acrolein precursor to be used will depend on the particular cell type to which the acrolein precursor is contacted. For example, in mouse hepatocytes the acrolein precursor may be allyl alcohol. In neuronal cells, the acrolein precursor may be allylamine.

Contacting the cells with the test molecule may be by a suitable method known in the art, including the direct contact of the test molecule with the cells.

The ability to detect acrolein-protein adducts and the ability to reverse the formation of adducts will depend on the respective concentrations of the protein molecule, acrolein or the acrolein precursor, and the test molecule.

As will also be appreciated, the test molecule will be contacted with the acrolein-protein adducts sometime after the reaction of acrolein (or acrolein precursor) with the protein molecule. Preferably, the test molecule is contacted within 2 hours of exposure to acrolein or the acrolein precursor. More preferably, the test molecule is contacted within 1 hour of exposure to acrolein or the acrolein precursor. Most preferably, the test molecule is contacted within 30 minutes of exposure to acrolein or the acrolein precursor.

The ability of a test molecule to reverse the formation of acrolein-protein adducts may be determined by the extent of reversible modification to one or more peptides, polypeptides or proteins exposed to acrolein (or the acrolein precursor). The detection of acrolein-modified proteins containing one or more acrolein adducts may be by a suitable procedure, such as detection utilising one or more suitable polyclonal or monoclonal antibodies raised to an acrolein-modified protein. Detection of the proteins may be by ELISA analysis, Western analysis, slot blot analysis or other methods of detecting proteins with the use of antibodies that are known in the art. Preferably, the antibody is a polyclonal antibody raised against acrolein-modified KLH. Alternatively, the detection of modified proteins may be by a method that includes mass spectrometry to detect adducts in the modified proteins.

Preferably, the acrolein-modified proteins detected for assessment of the reversible modification are proteins containing acrolein-lysine adducts. In one specific form, the acrolein-lysine adducts are FDP-lysine or one or more precursors to FDP-lysine. In this case, the contacting of the test molecule with the acrolein-lysine adduct occurs prior to the substantial formation of FDP-lysine.

In the case of this form of the present invention being performed in an in vitro cell-free system, acrolein-modified proteins that are reversibly modified may be prepared for analysis by incorporating the mixture into a buffer suitable for the loading of the sample onto a SDS/PAGE gel. More preferably, the buffer suitable for loading does not contain reducing agents, including mercaptoethanol and/or dithiothreitol. Preferably the buffer suitable for loading is not heated prior to loading on a SDS/PAGE gel.

The acrolein-modified proteins may then be analysed by resolving the proteins by SDS/PAGE and the use of a suitable antibody to detect the acrolein-modified proteins.

In the case of the method of this form of the invention being performed in cells, the step of detecting one or more acrolein-modified proteins that are reversibly modified includes a step of lysing sufficient cells to allow for detection of acrolein-modified proteins. Preferably, the lysing of cells includes a step of lysing the cells in a buffer that does not include an amine buffer. More preferably the lysing of cells includes a step of lysing the cells in a buffer that does not include a Tris buffer.

Further, the step of detecting in cells one or more acrolein-modified proteins that are reversibly modified may include a step of preparing a cell lysate for analysis on a SDS/PAGE gel by preparing a buffer suitable for the loading of the cell lysate sample onto the SDS/PAGE gel. More preferably, the buffer suitable for loading does not contain reducing agents, including mercaptoethanol and/or dithiothreitol. Preferably the buffer suitable for loading is not heated prior to loading on a SDS/PAGE gel.

The acrolein-modified proteins may then be analysed by resolving the proteins by SDS/PAGE and the use of a suitable antibody to detect the acrolein-modified proteins, as described above. Alternatively, the detection of modified proteins may be by a method that includes mass spectrometry to detect adducts in the modified proteins.

As will appreciated, the extent of reversibly modified proteins will be equal to the difference between the concentration of acrolein-modified products existing before treatment and the concentration of acrolein-modified products existing after treatment with the test molecule.

Preferably, the step of determining the extent of acrolein-modified proteins that are reversibly modified includes the step of detecting the concentration of proteins that include one or more FDP-lysine adducts (or one or more precursors to FDP-lysine). Once again, the concentration of reversibly modified proteins will be equal to the difference between the concentration of FDP-lysine products (or one or more precursors to FDP-lysine) existing before treatment and the concentration of FDP-lysine products (or one or more precursors to FDP-lysine) existing after treatment.

The present invention also provides a method of preventing and/or treating a disease or condition associated with damage mediated by an α,β-unsaturated aldehyde in a subject, the method including the step of administering to the subject a therapeutically effective amount of a hydrazino compound.

Preferably, the hydrazino compound is a compound with the following chemical formula:

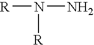

or a pharmaceutically acceptable salt thereof; wherein R is H; aryl; substituted aryl including hydrazino-substituted aryl, hydroxy-substituted aryl, and nitro-substituted aryl; heteroaryl; substituted heteroaryl including hydrazino-substituted heteroaryl, hydroxy-substituted heteroaryl, and nitro-substituted heteroaryl; benzyl; anilino; alkylbenzene; C₁ to C₈ alkyl; or C₅ to C₈ cycloalkyl.

Preferably, the hydrazino compound is selected from the group consisting of is selected from the group consisting of 1,1-diphenylhydrazine, hydrazinoisoquinoline, naphthylhydrazine, phenylhydrazine, hydrazinoquinazoline, hydrazinoquinoline, dihydralazine, hydralazine, 1,2-diphenylhydrazine, 2,4-dinitro-phenylhydrazine, benzylhydrazine, hydrazinopyridine, dimethylhydrazine, and aminoguanidine.

Preferably, the α,β-unsaturated aldehyde is acrolein, malondialdehyde, a 4-hydroxyalkenal, a dienal, a 2-alkenal, or the reactive a,b-unsaturated aldehyde tautomers of any of these compounds. Most preferably, the α,β-unsaturated aldehyde is acrolein.

Preferably, the disease or condition is a disease or condition associated with oxidative stress; a disease or condition associated with acute or chronic exposure to smoke; a disease or condition associated with the onset and/or progression of chronic and/or degenerative diseases associated with the ageing process; Alzheimer's disease; Parkinson's disease; Huntington's disease; a disease or condition associated with the onset and/or progression of central nervous indications including mild cognitive impairment and incipient dementia; neoplastic disease; a disease or condition associated with cell transformation; a neurodegenerative disease; a vascular disease including atherosclerosis and stroke; diabetes or complications of diabetes including diabetic renal disease; liver disease including alcoholic liver disease; ischemic tissue injury; a condition associated with cyclophosphamide chemotherapy including cyclophosphamide chemotherapy of the bladder, ovary, breast, cervix and lung cells; smoke-induced pulmonary oedema; or a disease or condition cells associated with dermal photo-damage.

Most preferably, the disease or condition is a neurodegenerative disease or condition, a condition associated with cyclophosphamide chemotherapy, or acute or chronic exposure to smoke.

The administration of the hydrazino compound may be within any time suitable to produce the desired effect.

For example, in the case of preventing a disease or condition associated with damage mediated by an α,β-unsaturated aldehyde, the compound may be administered prior to exposure to the α,β-unsaturated aldehyde, and/or prior to the damage mediated by the α,β-unsaturated aldehyde occurring.

In the case of treating a disease or condition, the hydrazino compound may be administered at any suitable time prior to, during, or after exposure of the subject to the α,β-unsaturated aldehyde, so long as the exposure is within a time period to reduce or prevent further damage mediated by the α,β-unsaturated aldehyde.

Preferably, administration occurs within 4 hours of exposure to an α,β-unsaturated aldehyde, more preferably within 2 hours, even more preferably within 1 hour, and most preferably within 30 minutes.

The amount of the hydrazino compound is not particularly limited, so long as it is within such an amount that generally exhibits a pharmacologically therapeutic effect. Preferably, the administration of the compound to a subject is in the range from 0.1 to 100 μmol/kg. Most preferably, the administration of the compound to a subject is in the range from 1 to 10 μmol/kg. The subject is preferably an animal or human subject.

The details of the administration of the hydrazino compound, and details of the formulation of a composition suitable for administration, are as previously discussed in relation to the administration and formulation of hydralazine and dihyralazine.

The present invention also provides a method of preventing and/or treating a disease or condition associated with damage mediated by an α,β-unsaturated aldehyde in a subject, the method including the step of administering to the subject a therapeutically effective amount of a compound with the following chemical formula:

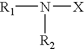

or a pharmaceutically acceptable salt thereof; wherein
X is NH₂ or H;
R₁ is aryl; substituted aryl including hydrazino-substituted aryl, hydroxy-substituted aryl, and nitro-substituted aryl; heteroaryl; substituted heteroaryl including hydrazino-substituted heteroaryl, hydroxy-substituted heteroaryl, and nitro-substituted heteroaryl; benzyl; anilino; alkylbenzene; C₁ to C₈ alkyl; C₅ to C₈ cycloalkyl; and
R₂ is aryl; substituted aryl; C₁ to C₈ alkyl; C₅ to C₈ cycloalkyl; or H.

Preferably the agent is selected 1,1-diphenylhydrazine, diphenylamine, N,N′-Diphenyl-p-phenylenediamine, N-methylbutylamine, hydrazinoisoquinoline, naphthylhydrazine, phenylhydrazine, hydrazinoquinazoline, hydrazinoquinoline, dihydralazine, hydralazine, o-aminophenol, 1,2-diphenylhydrazine, p-aminophenol 2,4-dinitro-phenylhydrazine, benzylhydrazine, carbazole, hydrazinopyridine, dicyclohexylamine, aniline, diisopropylamine, benzylamine, cyclohexylamine, p-hydroxyamphetamine, or dimethylhydrazine.

Preferably, the α,β-unsaturated aldehyde is acrolein, malondialdehyde, a 4-hydroxyalkenal, a dienal, a 2-alkenal, or the reactive a,b-unsaturated aldehyde tautomers of any of these compounds. Most preferably, the α,β-unsaturated aldehyde is acrolein.

Preferably, the disease or condition is a disease or condition associated with oxidative stress; a disease or condition associated with acute or chronic exposure to smoke; a disease or condition associated with the onset and/or progression of chronic and/or degenerative diseases associated with the ageing process; Alzheimer's disease; Parkinson's disease; Huntington's disease; a disease or condition associated with the onset and/or progression of central nervous indications including mild cognitive impairment and incipient dementia; neoplastic disease; a disease or condition associated with cell transformation; a neurodegenerative disease; a vascular disease including artherosclerosis and stroke; diabetes or complications of diabetes including diabetic renal disease; liver disease including alcoholic liver disease; ischemic tissue injury; a condition associated with cyclophosphamide chemotherapy including cyclophosphamide chemotherapy of the bladder, ovary, breast, cervix and lung cells; smoke-induced pulmonary oedema; or a disease or condition cells associated with dermal photo-damage.

Most preferably, the disease or condition is a neurodegenerative disease or condition, a condition associated with cyclophosphamide chemotherapy, or acute or chronic exposure to smoke.

The administration of the compound may be within any time suitable to produce the desired effect.

For example, in the case of preventing a disease or condition associated with damage mediated by an α,β-unsaturated aldehyde, the compound may be administered prior to exposure to the α,β-unsaturated aldehyde, and/or prior to the damage mediated by the α,β-unsaturated aldehyde occurring.

In the case of treating a disease or condition, the compound may be administered at any suitable time prior to, during, or after exposure of the subject to the α,β-unsaturated aldehyde, so long as the exposure is within a time period to reduce or prevent further damage mediated by the α,β-unsaturated aldehyde.

Preferably, administration occurs within 4 hours of exposure to an α,β-unsaturated aldehyde, more preferably within 2 hours, even more preferably within 1 hour, and most preferably within 30 minutes.

The amount of compound is not particularly limited, so long as it is within such an amount that generally exhibits a pharmacologically therapeutic effect. Preferably, the administration of the compound to a subject is in the range from 0.1 to 100 μmol/kg. Most preferably, the administration of the compound to a subject is in the range from 1 to 10 μmol/kg. The subject is preferably an animal or human subject.

The details of the administration of the compound, and details of the formulation of a composition suitable for administration, are as previously discussed in relation to the administration and formulation of hydralazine and dihyralazine.

The present invention also provides a method of inhibiting cross-linking of molecules by an α,β-unsaturated aldehyde, the method including the step of inhibiting formation of an adduct of a first molecule with an α,β-unsaturated aldehyde and/or inhibiting reaction of the adduct with a second molecule to cross-link the molecules.

In this regard, it has been found that α,β-unsaturated aldehydes also have the capacity to cross-link molecules. Without being bound by theory, it appears that an α,β-unsaturated aldehyde first reacts with a molecule to form an adduct between the α,β-unsaturated aldehyde and the molecule, and that subsequently a reactive group on the adduct reacts with another molecule, thereby cross-linking the molecules.

The formation of such cross-linked molecules is deleterious to cells. The method of this form of the present invention is therefore particularly useful for inhibiting the formation of cross-linked molecules in cells, by either inhibiting the initial formation of the adduct of an α,β-unsaturated aldehyde with a molecule (for example by scavenging the α,β-unsaturated aldehyde) and/or inhibiting the subsequent reaction of the adduct with another molecule to cross-link the molecules.

Preferably, the α,β-unsaturated aldehyde is acrolein, malondialdehyde, a 4-hydroxyalkenal, a dienal, a 2-alkenal, or the reactive α,β-unsaturated aldehyde tautomers of any of these compounds. Most preferably, the α,β-unsaturated aldehyde is acrolein.

Preferably, the inhibition of reaction of the adduct with the second molecule to cross-link the first and second molecules involves inhibition of the reaction of a carbonyl group on the adduct with a reactive group on the second molecule.

The first molecule may be any nucleophilic molecule capable of reacting with the α,β-unsaturated aldehyde, including a protein, polypeptide, or a nucleic such as DNA, mRNA, rRNA and tRNA. Preferably, the first molecule is a protein.

The second molecule is any molecule capable of being cross-linked to the first molecule by the reaction of the α,β-unsaturated aldehyde-adduct with a reactive group on the second molecule. Preferably, the second molecule is a protein or a nucleic acid. Most preferably, the second molecule is a protein.

Thus, the method of the present invention is applicable to the inhibition of formation of protein-protein cross links and protein-nucleic acid cross-links by α,β-unsaturated aldehydes, including the inhibition of protein-DNA cross-links. However, it will also be appreciated that intra-molecular cross-linking is also included within the scope of this form of the present invention.

The cross-linking reaction may occur either in vitro in a cell free system, in cells in vitro, or in vivo.

In the case of an adduct of the α,β-unsaturated aldehyde with a protein, preferably the adduct of the α,β-unsaturated aldehyde is with a lysine residue in the protein.

Preferably, the inhibition of cross-linking may be achieved by exposing the molecules to be cross-linked to an agent that can scavenge the α,β-unsaturated aldehyde and thereby reduce the rate of reaction of the α,β-unsaturated aldehyde with a molecule, and/or react with an existing adduct of a molecule with an α,β-unsaturated aldehyde and thereby prevent cross-linking to another molecule. Accordingly, the inhibition of cross-linking in this form of the present invention preferably includes exposure of the first molecule to an agent that inhibits adduct formation and/or inhibits reaction of the adduct with a second molecule.

In the case of the agent that reacts with the adduct to prevent cross-linking, preferably the agent reacts with a carbonyl group on the adduct to inhibit the carbonyl group reacting with a reactive group on the second molecule.

Preferably, the agent is a hydrazino compound. More preferably, the hydrazino compound is a compound with the following chemical formula:

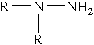

or a pharmaceutically acceptable salt thereof; wherein R is H; aryl; substituted aryl including hydrazino-substituted aryl, hydroxy-substituted aryl, and nitro-substituted aryl; heteroaryl; substituted heteroaryl including hydrazino-substituted heteroaryl, hydroxy-substituted heteroaryl, and nitro-substituted heteroaryl; benzyl; anilino; alkylbenzene; C₁ to C₈ alkyl; or C₅ to C₈ cycloalkyl.

Most preferably, the hydrazino compound is selected from the group consisting of is selected from the group consisting of 1,1-diphenylhydrazine, hydrazinoisoquinoline, naphthylhydrazine, phenylhydrazine, hydrazinoquinazoline, hydrazinoquinoline, dihydralazine, hydralazine, 1,2-diphenylhydrazine, 2,4-dinitro-phenylhydrazine, benzylhydrazine, hydrazinopyridine, dimethylhydrazine, and aminoguanidine.

The method of this form of the present invention may be used to inhibit cross-linking an in vitro cell free system, or in a biological system. Preferably, the biological system is an animal or human.

In the case of a human subject, preferably the method of this form of the present invention is used to inhibit the cross-linking of molecules in a human susceptible to, or suffering from, a disease or condition associated with oxidative stress; a disease or condition associated with acute or chronic exposure to smoke; a disease or condition associated with the onset and/or progression of chronic and/or degenerative diseases associated with the ageing process; Alzheimer's disease; Parkinson's disease; Huntington's disease; a disease or condition associated with the onset and/or progression of central nervous indications including mild cognitive impairment and incipient dementia; neoplastic disease; a disease or condition associated with cell transformation; a neurodegenerative disease; a vascular disease including artherosclerosis and stroke; diabetes or complications of diabetes including diabetic renal disease; liver disease including alcoholic liver disease; ischemic tissue injury; a condition associated with cyclophosphamide chemotherapy including cyclophosphamide chemotherapy of the bladder, ovary, breast, cervix and lung cells; smoke-induced pulmonary oedema; or a disease or condition cells associated with dermal photodamage.

Therefore, the inhibition of cross-linking may be used to reduce damage mediated by an α,β-unsaturated aldehyde in a biological system.

Accordingly, in another form the present invention provides a method of reducing damage mediated by an α,β-unsaturated aldehyde in a biological system, the method including the step of administering to the biological system an effective amount of an agent that inhibits cross-linking of molecules by the α,β-unsaturated aldehyde in the biological system.

As discussed previously, it will be appreciated that the damage may be mediated by the endogenous production of an α,β-unsaturated aldehyde in the biological system, or alternatively, may be due to the production of α,β-unsaturated aldehyde in the biological system by exposure to exogenous agents, such as smoke or the exposure of the biological system to cyclophosphamide chemotherapy, both of which result in the production of acrolein.

Damage mediated by an α,β-unsaturated aldehyde may be measured in a suitable manner that is known in the art, and applicable to the biological system being assessed. Damage will be understood to mean any deleterious effect arising from endogenous production of an α,β-unsaturated aldehyde, any deleterious effect arising from exogenous α,β-unsaturated aldehyde exposure, or any deleterious effect arising from exposure to a precursor of an α,β-unsaturated aldehyde. One measure of damage is cellular toxicity, which may be measured for example using probes for membrane integrity, cellular metabolic status or mitochondrial activity. For example, toxicity may be measured by the extent of leakage of a molecule from a cell or by the presence of an enzyme marker that is diagnostic of α,β-unsaturated aldehyde toxicity. In the case of damage mediated by acrolein, toxicity may be measured for example by the extent of leakage of LDH from a cell or the activity of the enzyme sorbitol dehydrogenase.

The administration of the agent may be within any time suitable to produce the desired effect. Preferably, administration occurs within 4 hours of exposure to an α,β-unsaturated aldehyde, more preferably within 2 hours, even more preferably within 1 hour, and most preferably within 30 minutes.

The amount of agent is not particularly limited, so long as it is within such an amount that generally exhibits a pharmacologically therapeutic effect. Preferably, the administration of the agent to a subject is in the range from 0.1 to 100 μmol/kg. Most preferably, the administration of the agent to a subject is in the range from 1 to 10 μmol/kg. The subject is preferably an animal or human subject.

The details of the administration of the agent, and details of the formulation of a composition suitable for adminstration, are as previously discussed in relation to the administration and formulation of hydralazine and dihyralazine.

The reduction of damage by the agent may be used to prevent and/or treat a condition in a subject that is associated with damage mediated by an α,β-unsaturated aldehyde.

Accordingly, in another form the present invention provides a method of preventing and/or treating a disease or condition associated with damage mediated by an α,β-unsaturated aldehyde in a subject, the method including the step of administering to the subject a therapeutically effective amount of an agent that inhibits cross-linking of molecules by the α,β-unsaturated aldehyde.

Preferably, the method is useful for preventing and/or treating a neurodegenerative disease, preventing and/or treating the effects of cyclophosphamide chemotherapy, or preventing and/or treating the effects of acute or chronic exposure to smoke.

The present invention also provides a method of identifying a molecule that inhibits cross-linking of molecules by an α,β-unsaturated aldehyde, the method including the steps of:
(a) exposing a substrate to an α,β-unsaturated aldehyde;
(b) determining the ability of a test molecule to inhibit cross-linking of the substrate by the α,β-unsaturated aldehyde; and
(c) identifying the test molecule as a molecule that inhibits cross-linking of molecules by an α,β-unsaturated aldehyde by the ability of the test molecule to inhibit cross-linking of the substrate.

Preferably, the α,β-unsaturated aldehyde is acrolein, malondialdehyde, a 4-hydroxyalkenal, a dienal, a 2-alkenal, or the reactive α,β-unsaturated aldehyde tautomers of any of these compounds. Most preferably, the α,β-unsaturated aldehyde is acrolein.

The substrate may be any substrate that may be cross-linked by an α,β-unsaturated aldehyde. Preferably, the substrate is a protein or a nucleic acid. Most preferably, the substrate is a protein.

In a particularly preferred form, the cross-linking of the substrate is cross-linking of the protein to another protein or cross-linking of the protein to a nucleic acid.

The exposure of the substrate to an α,β-unsaturated aldehyde may occur in an in vitro cell free system, in cells in vitro, or in vivo. Examples of suitable biological systems are as previously discussed.

Identification of the cross-linked substrate may be by a suitable method known in the art. For example, in the case where the substrate is a protein, Western Blot analysis with a specific antibody to a particular protein and observing the inhibition of formation of higher molecular weight species may be used.

The present invention also provides a molecule identified according to the method of this form of the present invention. Molecules so identified are likely candidates for reducing damage mediated by an α,β-unsaturated aldehyde in a biological system.

Accordingly, in a preferred from the present invention provides a method of identifying a molecule that reduces damage mediated by an α,β-unsaturated aldehyde in a biological system, the method including the step of identifying a molecule that inhibits cross-linking of molecules by an α,β-unsaturated aldehyde.

The present invention also provides an antibody (or an antigen-binding portion thereof) that binds to an α,β-unsaturated aldehyde-hydrazino compound adduct.

The antibody may be a monoclonal or a polyclonal antibody. The antibody may be an isolated antibody.

An antibody is an intact immunoglobulin. An immunoglobulin is a tetrameric molecule, each tetramer being composed of two identical pairs of polypeptide chains, each pair having one light chain and one heavy chain. The amino-terminal portion of each chain includes a variable region of about 100 to 110 or more amino acids that is primarily responsible for antigen recognition. The carboxy-terminal portion of each chain defines a constant region primarily responsible for effector function. Human light chains are classified as κ and λ light chains. Heavy chains are classified as μ, Δ, γ, α, or ε and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. The variable regions of each light/heavy chain pair form the antibody binding site, with the result that an intact immunoglobulin has two binding sites.

The antigen-binding portion of an antibody molecule includes a Fab, Fab′, F(ab′)₂, Fv, a single-chain antibody (scFv), a chimeric antibody, a diabody or any polypeptide that contains at least a portion of an immunoglobulin that is sufficient to confer specific antigen binding.

A Fab fragment is a monovalent fragment consisting of the V_{L}, V_{H}, C_{L} and C_{H} I domains. A F(ab′)₂ fragment is a bivalent fragment including two Fab fragments linked by a disulphide bridge at the hinge region. A Fd fragment consists of the V_{H} and C_{H} I domains. A Fv fragment consists of the V_{L} and V_{H} domains of a single arm of an antibody. A dAb consists of a V_{H} domain.

A single chain antibody (scFv) is an antibody in which V_{L} and V_{H} regions are paired to form a monovalent molecule via a synthetic linker that enable them to be made as a single protein chain. Diabodies are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites.

It will be understood that the antibody and antigen-binding portions also include humanized antibodies and antigen-binding portions thereof, in which amino acids have been replaced in the non-antigen binding regions in order to more closely resemble a human antibody, while still retaining the original binding ability.

Preferably, the hydrazino compound is a compound with the following chemical formula:

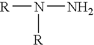

or a pharmaceutically acceptable salt thereof; wherein R is H; aryl; substituted aryl including hydrazino-substituted aryl, hydroxy-substituted aryl, and nitro-substituted aryl; heteroaryl; substituted heteroaryl including hydrazino-substituted heteroaryl, hydroxy-substituted heteroaryl, and nitro-substituted heteroaryl; benzyl; anilino; alkylbenzene; C₁ to C₈ alkyl; or C₅ to C₈ cycloalkyl.

Preferably, the hydrazino compound is selected from the group consisting of is selected from the group consisting of 1,1-diphenylhydrazine, hydrazinoisoquinoline, naphthylhydrazine, phenylhydrazine, hydrazinoquinazoline, hydrazinoquinoline, dihydralazine, hydralazine, 1,2-diphenylhydrazine, 2,4-dinitro-phenylhydrazine, benzylhydrazine, hydrazinopyridine, dimethylhydrazine, and aminoguanidine. Most preferably, the hydrazino compound is hydralazine.

Preferably, the α,β-unsaturated aldehyde is acrolein, malondialdehyde, a 4-hydroxyalkenal, a dienal, a 2-alkenal, or the reactive a,b-unsaturated aldehyde tautomers of any of these compounds. Most preferably, the α,β-unsaturated aldehyde is acrolein.

The adduct is preferably an adduct of a protein, a polypeptide, or a nucleic such as DNA, mRNA, rRNA and tRNA. Preferably, the adduct is an adduct with a protein or a polypeptide.

Preferably, the antibody is an antibody to an α,β-unsaturated aldehyde-hydralazine protein or polypeptide adduct, or an antibody to an α,β-unsaturated aldehyde-dihydralazine protein or polypeptide adduct.

More preferably, the antibody is an antibody to an acrolein-hydralazine protein or polypeptide adduct, or an antibody to an acrolein-dihydralazine protein or polypeptide adduct.

For example, in the case of an acrolein-hydralzine protein or polypeptide adduct, the antibody may also be raised against any protein or polypeptide that contains an acrolein-hydralazine adduct. In this regard, preferably, the acrolein-hydralazine adduct is formed by first reacting a protein with acrolein and subsequently treating the acrolein-protein adduct with hydralazine.

In this way, hydralazine may react with a reactive carbonyl group formed upon the reaction of the acrolein with the protein. Preferably, the hydralazine acrolein addcut is a mono-ACR-[protein/polypeptide]-HYD adduct, a FDP-[protein/polypeptide]-HYD adduct, or a bis-ACR-[protein/polypeptide]-HYD addict, the structures of which have been experimentally confirmed as shown in Example 14, and as shown in scheme IV below:

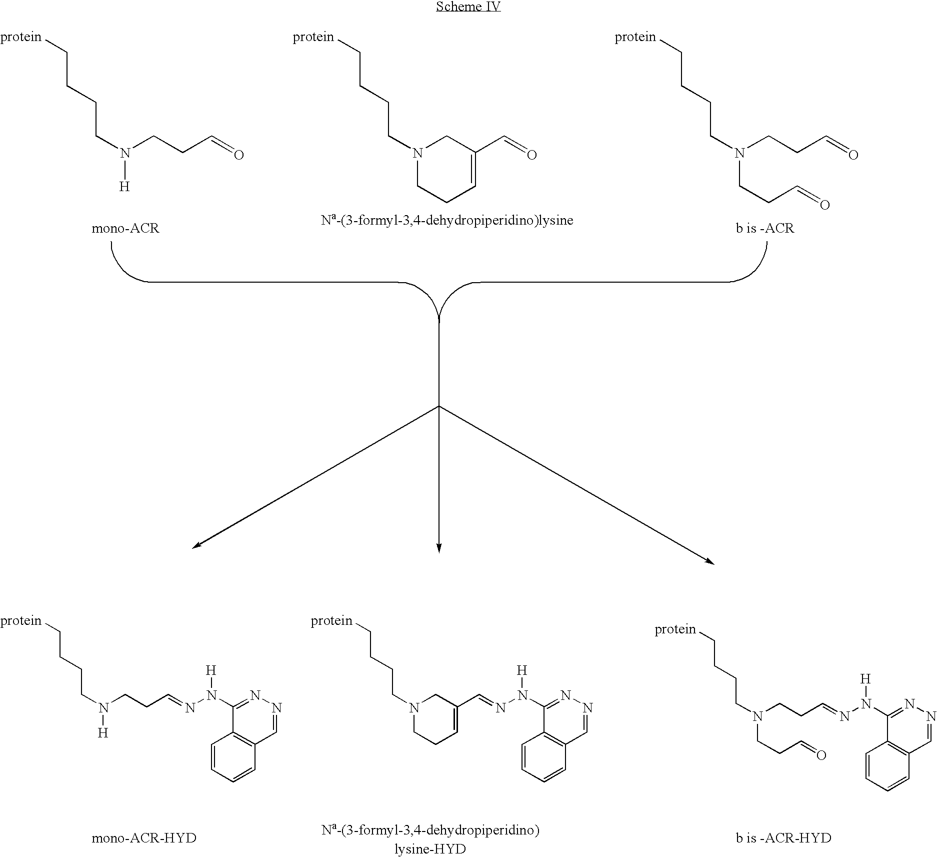

Antibodies to an acrolein-hydralazine/dihydralzine protein/polypeptide adduct may be generated using methods known in the art. For the production of antibodies, various hosts including goats, rabbits, rats, mice, humans, and others, may be immunized by injection with the adduct. Depending on the host species, various adjuvants may be used to increase immunological response. Such adjuvants include Freund's adjuvant, mineral gels such as aluminium hydroxide, and surface-active substances such as lysolecithin, pluronic polyols, polyanions, peptides, oil emulsions, keyhole limpet hemocyanin, and dinitrophenol.

A polyclonal antibody is an antibody that is produced among or in the presence of one or more other, non-identical antibodies. In general, polyclonal antibodies are produced from B-lymphocytes. Usually, polyclonal antibodies are obtained directly from an immunized subject, such as an immunized animal.

Monoclonal antibodies may be prepared using any technique that provides for the production of antibody molecules by continuous isolated cells in culture. These include, but are not limited to, the hybridoma technique, the human B-cell hybridoma technique, and the EBV-hybridoma technique. Methods for the preparation of monoclonal antibodies are as generally described in Kohler et al. (1975) Nature 256:495-497, Kozbor et al. (1985) J. Immunol. Methods 81:31-42, Cote et al. (1983) Proc. Natl. Acad. Sci. 80:2026-2030, and Cole et al. (1984) Mol. Cell Biol. 62:109-120.

Accordingly, in another preferred form the present invention provides an isolated cell that produces an antibody to an acrolein-hydralazine protein/polyeptide adduct, or an antigen-binding portion of the antibody.

Humanized antibodies, or antibodies adapted for non-rejection by other mammals, may also be produced by a suitable method known in the art, such as resurfacing or CDR grafting.

Techniques developed for the production of “chimeric antibodies”, for example the splicing of mouse antibody genes to human antibody genes to obtain a molecule with appropriate antigen specificity and biological activity, may be performed by a suitable method known in the art.

Antibody fragments that contain specific binding sites may be generated by methods known in the art. For example, F(ab′)₂ fragments can be produced by pepsin digestion of the antibody molecule, and Fab fragments can be generated by reducing the disulfide bridges of the F(ab′)₂ fragments. Alternatively, Fab expression libraries may be constructed to allow rapid and easy identification of monoclonal Fab fragments with the desired specificity..

Included within the scope of the antibodies and antigen-binding portions of the various forms of the present invention are also variants of the antibodies and their antigen-binding portions of the present invention. For example, variants include polypeptides with amino acid sequences that are similar to the amino acid sequence of the variable or hypervariable regions of the antibodies of the present invention.

Preferably, the variants have at least about 90%, and more preferably at least about 95% sequence identity to another amino acid sequence, as determined by the FASTA search method, as described in Pearson and Lipman (1988) Proc. Natl. Acad. Sci. USA 85: 2444-2448.

Various immunoassays may be used for screening to identify antibodies having the desired specificity. Numerous protocols for competitive binding or immunoradiometric assays using either polyclonal or monoclonal antibodies are known in the art.

The antibody molecules may also be produced recombinantly by methods known in the art, for example by expression in E.coli/T7 expression systems. A suitable method for the production of recombinant antibodies is as described in U.S. Pat. No. 4,816,567.

Such antibodies are useful as diagnostic reagents to detect hydralazine-acrolein adducted proteins and polypeptides. Various methods are also known in the art for using antibodies to detect proteins, as described generally in Sambrook, J, Fritsch, E. F. and Maniatis, T. Molecular Cloning: A Laboratory Manual 2nd. ed. Cold Spring Harbor Laboratory Press, New York. (1989).

The present invention also provides a method of determining the extent of damage mediated by an α,β-unsaturated aldehyde in a biological system, the method including the step of determining the concentration of one or more molecules in the biological system that are cross-linked to another molecule by an α,β-unsaturated aldehyde.

Methods of determining the extent of damage mediated by α,β-unsaturated aldehydes in the various forms of the present invention are useful as diagnostic tests to determine the extent of damage due to these agents in a biological system.

Preferably, the α,β-unsaturated aldehyde is acrolein, malondialdehyde, a 4-hydroxyalkenal, a dienal, a 2-alkenal, or the reactive α,β-unsaturated aldehyde tautomers of any of these compounds. Most preferably, the α,β-unsaturated aldehyde is acrolein.

Preferably, the biological system includes hepatocyte cells; neuronal cells; lung epithelial cells; cells undergoing oxidative stress; cells having been exposed to smoke; cells associated with the onset and/or progression of chronic and/or degenerative diseases associated with the ageing process; cells associated with the onset and/or progression of Alzheimer's disease, Parkinson's disease or Huntington's disease; cells associated with the onset and/or progression of central nervous indications including mild cognitive impairment and incipient dementia; cells associated with neoplastic diseases or cell transformation; cells associated with neurodegenerative diseases; cells associated with vascular diseases including atherosclerosis and stroke; cells associated with diabetes or complications of diabetes including diabetic renal disease; cells associated with liver disease including alcoholic liver disease; cells associated with ischemic tissue injury; cells susceptible to injury during cyclophosphamide chemotherapy including cyclophosphamide chemotherapy of bladder, ovary, breast, cervix and lung cells; cells susceptible to damage due to acute or chronic smoke inhalation including gingivial; cells associated with smoke-induced pulmonary oedema; or cells associated with dermal photodamage.

More preferably, the biological system is an animal or human.

The determination of the concentration of one or molecules in the biological system that are cross-linked to another molecule by the α,β-unsaturated aldehyde may be by a suitable method known in the art, such as the use of an antibody to detect cross-linked molecules.

### DESCRIPTION OF PREFERRED EMBODIMENTS

Reference will now be made to experiments that embody the above general principles of the present invention. However, it is to be understood that the following description is not to limit the generality of the above description.

### EXAMPLE 1

Reaction of Hydralazine and Dihydralazine with Acrolein

To assess the rate of reaction between acrolein and test acrolein trapping compounds hydralazine, dihydralazine, pyridoxamine, aminoguanidine, methoxyamine and carnosine in a protein-free system, acrolein (0.5 mM) was added to prewarmed solutions of the above test compounds (0.5 mM) dissolved in buffer (50 mM sodium phosphate, pH 7.0). Reactions proceeded with mixing at 37° C. At 10 minute intervals 100 μL aliquots were removed and diluted in mobile phase before they were injected immediately onto a HPLC system which comprised a C-18 column (SGE, Exsil ODS2, 5 μm, 150 mm×4.6 mm) that was eluted at 1 mL/min with 20% methanol in water. The absorbance of the eluent was monitored at 210 nm using a Hewlett Packard 1100 UV/Vis detector. The retention time for acrolein under these conditions was 2.2 minutes. Levels of acrolein in the reaction mixtures were determined using a standard curve prepared from freshly made solutions of acrolein.

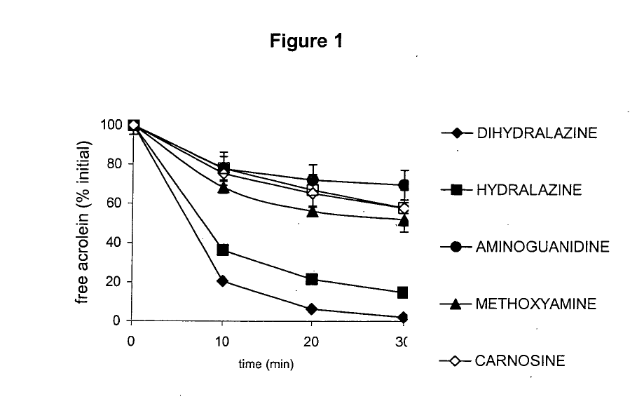

The data obtained is shown in FIG. 1. Each data point represents the mean±S.E. of 3 independent observations. The data shows that the rate of reaction of the six potentially nucleophilic amine compounds with acrolein at 37° C. at neutral pH differs markedly. The data clearly indicates that the two hydrazinophthalazine drugs, hydralazine and dihydralazine, are highly efficient scavengers of acrolein. Within 30 minutes, both hydralazine and dihydralazine consumed greater than 90% of the acrolein.

### EXAMPLE 2

Hydralazine and Dihydralazine Reduce the Toxicity of Allyl Alcohol in Mouse Hepatocytes

To determine whether the acrolein-trapping properties of hydralazine and dihydralazine are relevant in a cellular model of acrolein-mediated toxicity, their effects on the toxicity of allyl alcohol in mouse hepatocytes was studied.

Allyl alcohol is rapidly oxidised to acrolein in liver cells by alcohol dehydrogenase, and causes pronounced cell death and protein modification (carbonylation). In this case the enzyme LDH leaks from cells that contain a ruptured membrane. Such leakage of LDH is a widely used indicator of cell death.

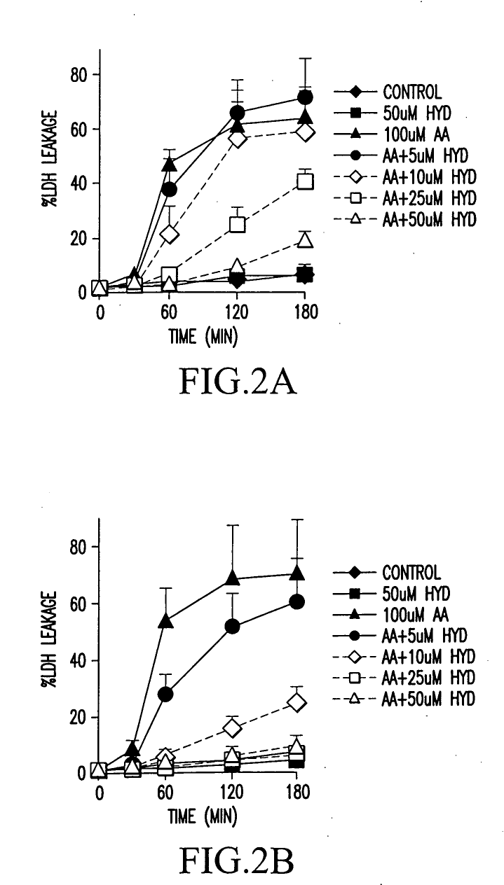

Various concentrations of each of the six abovementioned amine compounds was added to the culture media of mouse hepatocytes along with a toxic concentration of allyl alcohol (100 μM). Cells were placed in a humidified 5% CO₂ incubator and then aliquots of cell media were taken for the determination of lactate dehydrogenase (LDH) activity at 30 minute intervals. It was found that hydralazine and dihydralazine (up to 50 μM concentrations) strongly attenuated the toxicity of allyl alcohol in the cells, as shown in FIG. 2. Each data point represents the mean±S.E. of 4 independent observations.

### EXAMPLE 3

Hydralazine and Dihydralazine Show Pronounced Cytoprotective Activity in Mouse Hepatocytes Treated with Allyl Acohol

The cytoprotective potency of hydralazine and dihydralazine during allyl alcohol toxicity in mouse hepatocytes was compared to the activity of the four amine compounds methoxyamine, aminoguanidine, pyridoxamine and carnosine. The cytoprotective potency for each of the six compounds tested is shown in Table 1. Potencies are reported as PC₅₀ values, ie. concentrations affording 50% reduction in cell killing after a 1-hour co-exposure of cells to 100 μM allyl alcohol.

TABLE I
PC₅₀ Values* for Various Amine Compounds Against Allyl alcohol-
Induced Toxicity in Mouse Hepatocytes (N = 3, mean ± SE)

| | | |
|---|---|---|
| Hydralazine | 8.3 ± 4.0 | μM |
| Dihydralazine | 5.2 ± 1.5 | μM |
| Methoxyamine | 9.0 ± 1.8 | mM |
| Aminoguanidine | >10 | mM |
| Pyridoxamine | >10 | mM |
| Carnosine | >10 | mM |

It was found that both hydralazine and dihydralazine yielded PC₅₀'s that were over 3 orders of magnitude lower than those exhibited by the other nucleophilic amines examined (e.g. aminoguanidine, pyridoxamine, carnosine, methoxyamine). Although hydralazine and dihydralazine were effective at trapping acrolein (as shown in Example 1) the magnitude of the difference between the PC₅₀'s of hydralazine and dihydralazine and the remaining compounds indicated that additional effects were responsible for the ability of hyrdralazine and dihydralazine to cytoprotect against exposure to allyl alcohol.

### EXAMPLE 4

Preparation of a Rabbit Polyclonal Antibody to Acrolein-modified KLH

A rabbit polyclonal antibody was prepared by immunising rabbits with acrolein-modified protein Keyhole Limpet Hemocyanin (KLH). The immunogen was prepared by reacting KLH for 18 hours at 37° C. with 10 mM acrolein. The acrolein modified protein Was diluted with Freunds Complete Adjuvant and used to immunize a NZ White rabbit (1 mg/animal, 10 subcutaneous injection sites, 0.1 mg/site). The rabbit received seven subsequent booster injections with the immunogen at three weekly intervals. Two weeks after the final boost the animal was sacrificed and bled and serum recovered.

The antiserum was shown to detect acrolein adducts at lysine groups with high specificity and sensitivity. Confirmation that acrolein-modified lysine groups are the epitope for the antiserum was obtained by performing competitive inhibition experiments using acrolein-modified polyamino acids. These were prepared by reacting polyhistidine or polylysine with a concentration of acrolein that was double the concentration of nucleophilic amine monomers in the reaction mixtures (ie. The acrolein concentration was related to the average number of monomeric amino acids per amino acid polymer). Reactions with acrolein were performed at a concentration of 10 mg/mL polyamino acid in 50 mM sodium phosphate buffer (pH 7.0). Reactions were allowed to proceed for 16-18 hrs at 37° C. The modified polyamino acids were then dialysed against phosphate buffer for 24 hrs with several buffer changes, in a step that removed unreacted acrolein. Pierce 3.5 kDa-cut-off Slidealyser devices were used in this step. The ability of the modified polyamino acids to block immunorecognition of acrolein adducts in acrolein-treated BSA was then examined using a multichannel blotting device. The polyamino acids were added to the primary antibody solution (1/1000 dilution of rabbit antiserum in phosphate-buffered saline (PBS) containing 5% nonfat milk) at concentrations ranging from 0.01 to 1 mg/mL. The Western blot method described below was then used to complete the experiments. Acrolein- modified polylysine was a highly potent inhibitor of the immunorecognition of acrolein-modified BSA, while acrolein-modified polyhistidine, polylysine and polyhistidine lacked any inhibitory effects. This indicated that acrolein-adducted lysine are the epitope for this antibody.

### EXAMPLE 5

Hydralazine Lowers the Concentration of Acrolein Modified Proteins in Mouse Hepatocytes

The effect of hydralazine on the concentration of acrolein modified proteins in untreated mouse hepatocytes and mouse hepatcytes treated with various concentrations of acrolein was tested.

Mouse hepatocytes were exposed for 15 minutes to acrolein alone (0.5 mM) in the presence and absence of various concentrations of hydralazine: 0, 0.3, 1.0 or 3.0 mM. Cell lysates were then prepared before proteins were resolved on a 4% to 20% polyacrylamide gradient gel. Cell lysates were prepared by adding a small volume of Lysis Buffer to hepatocyte monlayers (eg. for a 60 mm dish containing 3 million liver cells, the volume of Lysis Buffer used was 0.4 mL). The Lysis Buffer contained sodium phosphate buffer (25 mM, pH 6.8), the nonionic detergent Nonidet P-40 (1% final concentration), 0.1% SDS, glycerol (20%), 10 mM EDTA and Sigma Protease Inhibitor Cocktail (0.5% final dilution). The composition of the Lysis Buffer was an important determinant of assay outcome, and care was taken to avoid including the amine buffer Tris in the mixture, as adducts were unstable to this reagent, particularly upon freezing of samples. Due to adduct instability issues, optimal assay outcomes are obtained if samples are immediately analysed upon the day of lysate preparation, with no effort to freeze the lysates before SDS/PAGE and subsequent steps.

To analyse proteins, the lysates were diluted with SDS/PAGE Loading Buffer and loaded onto polyacrylamide gels, with 50 to 80 μg protein loaded per lane. Note that while the Loading Buffer contained tris buffer (25 mM, pH 6.8), it did not contain reducing agents such as 2-mercaptoethanol or dithiothreitol. The samples also were not heated prior to gel loading. Although reducing agents and heating are commonly used to denature proteins prior to SDS/PAGE, it was found that acrolein-lysine adducts are unstable to these treatments.

After resolution on a minigel using conventional SDS/PAGE procedures, the proteins were transferred to reinforced nitrocellulose using the submerged tank method of electrophoretic transfer. A transfer buffer comprising tris/glycine (3.03 g and 14.4 g per litre, respectively) and 10% methanol produced optimal results (100 V, 40 mins). The nitrocellulose membrane was then blocked for 30 min in PBS containing 5% nonfat milk, before the primary antibody (rabbit anti-acrolein/KLH antiserum) was added at a dilution of 1/1000. After allowing immunorecognition to proceed for 60 mins at room temperature, the membranes were then washed extensively (3× with PBS, then 1× with tris-buffered saline, TBS, 5-10 min per wash with vigorous mixing). The secondary antibody step was then performed using peroxidase-coupled goat anti-rabbit IgG serum (Pierce Immunopure). The secondary antibody was used at a dilution of 1/10000, with the immunorecognition allowed to proceed for 30 mins. The membranes were then washed again using the same protocol described above. The membranes were finally treated for 5 min with Pierce PICO SuperSignal Chemiluminescence reagent before they were exposed to KODAK BioLight film for 5 to 15 mins before they were developed.

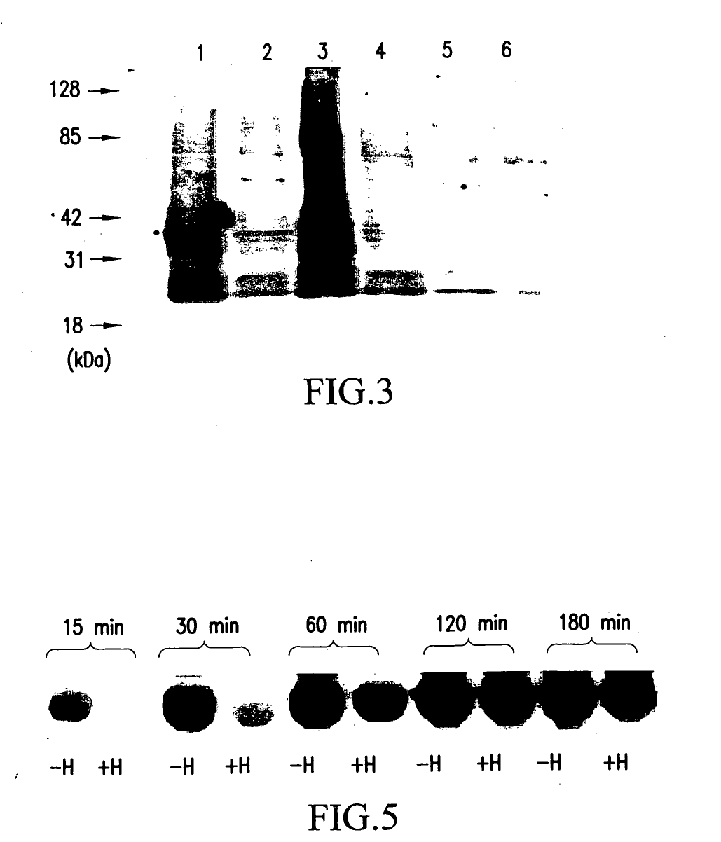

The results are shown in FIG. 3. The results show that a high level of adducts were evident in a number of proteins recovered from control cells, presumably as a result of endogenous lipid peroxidation (Lane 1). Treatment with acrolein alone strongly increased the immunostaining of a wide range of proteins (Lane 3). Unexpectedly, in cells exposed to hydralazine only (3 mM hydralazine, Lane 2) or acrolein (0.5 mM) plus various concentrations of hydralazine (0.3 to 3 mM, Lanes 4 to 6), the intensity of the adduct-containing bands was much lower than that seen in control cells. These experiments indicated that hydralazine may have the capacity to “break” bonds involved in the adduction of lysine residues by acrolein.

This data provides an explanation as to the unexpected disparity between the cytoprotective potency of hydralazine and dihydralazine and the other amine compounds tested in Example 3.

### EXAMPLE 6

Hydralazine and Dihydralazine Show the Ability to Reverse Adduct Formation In Vitro

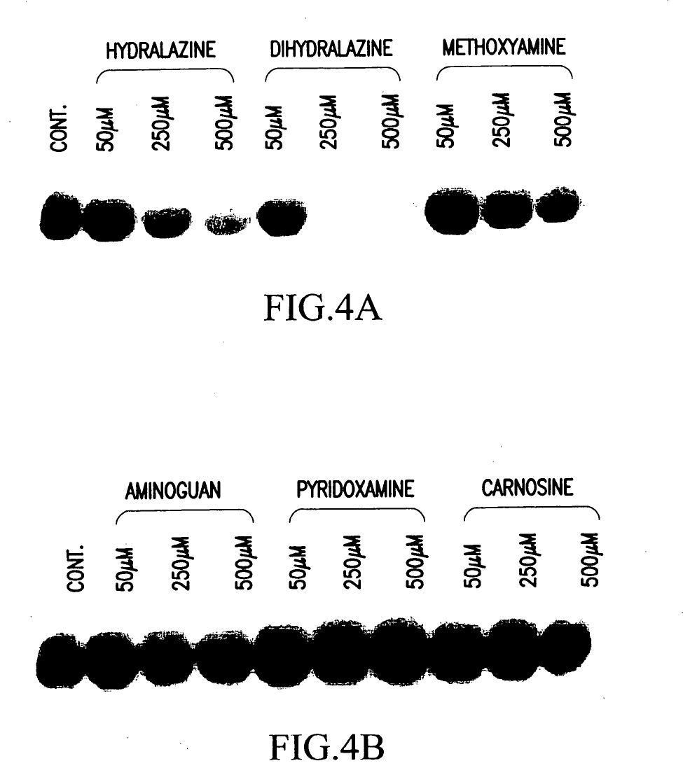

A simple in vitro, cell-free immunoassay was developed to aid screening compounds for an ability to achieve “adduct-breaking” at acrolein-modified lysines. For these experiments, a model protein (BSA, bovine serum albumin) was treated briefly with acrolein (1 mM, 20 mins) before it was reacted with various concentrations of scavengers in an “adduct-breaking” incubation (30 min at 37° C.). BSA (20 μg/lane) was then resolved via SDS/PAGE before it was transferred to nitrocellulose and subjected to “adduct detection” in a Western blot procedure using the acrolein-modified antibody decribed in Example 4 and the procedure for analysing modified proteins as described in Example 5. This assay allowed the comparison of the “adduct-breaking” potency of the amine compounds hydralazine, dihydralazine, methoxyamine, aminoguanidine, pyridoxamine and carnosine studied in preceding experiments. The test compounds were all studied at the same concentrations (50, 250 and 500 μM). The results from a representative experiment are shown in FIG. 4.

As in experiments in intact cells, hydralazine diminished the intensity of acrolein adducted-BSA in a concentration-dependent manner (Panel A). Dihydralazine displayed even greater potency in this regard, with the two highest concentrations of the drug reducing adducts below detectable levels (Panel A). Methoxyamine also displayed “adduct-breaking” actions in this assay, although the effects were most evident at the top concentration studied (Panel A). In contrast, neither aminoguanidine, pyridoxamine nor carnosine displayed any “adduct-breaking” activity in this assay (Panel B). Collectively, these findings are consistent with the cytoprotective potencies of these compounds summarised in Table I. Thus the three compounds that displayed no cytoprotective effects in hepatocytes (carnosine, pyridoxamine and aminoguanidine) also lacked adduct-breaking potency, whilst hydralazine and dihydralazine were very active in both assays. Methoxyamine was intermediate between these two poles in both assays.

### EXAMPLE 7

Time Course of Susceptibility of Acrolein Adducts to Hydralazine

Based on kinetic considerations, Michael addition reactions may proceed more rapidly than the subsequent ring forming and dehydration steps. Also, on chemical grounds, the two cyclic adducts formed in the final stages of the reaction sequence may be more stable than the early Michael adducts. Given these considerations, it was possible that the susceptibility of acrolein-modified lysine groups to nucleophilic attack by hydralazine would be greatest in the early stages of reactions with acrolein, but that they would become refractory to the drug upon formation of the stable, cyclised adducts (eg. FDP-lysine).

To determine the time-course of susceptibility to adduct-breaking by hydralazine, the effect of hydralazine on acrolein-modified BSA with time was examined. BSA was incubated with 1 mM acrolein for 15, 30, 60, 120 or 180 minutes before an excess of hydralazine (2 mM) was added. After a 30 min “adduct-breaking” reaction the protein was resolved via SDS/PAGE (20 μg BSA/lane) and then subjected to the immunoassay for acrolein-lysine adducts using the polyclonal antibody to acrolein modified KLH. The results from this experiment are shown in FIG. 5.

As can been seen in FIG. 5, the results confirmed the expectation that the susceptibility of acrolein-lysine adducts to hydralazine diminished with the progress of the reaction. In this experiment, acrolein-adducts retained their susceptibility to cleavage by hydralazine for at least 30 minutes and to some extent for 60 minutes.

### EXAMPLE 8

Hydralazine Displays Concentration Dependent Cytoprotective Potency During Adduction and Post-adduction Phases of Allyl Alcohol Toxicity in Mouse Hepatocytes

To determine whether the “adduct-breaking” actions of hydralazine are relevant at lower drug concentrations that are of greater clinical relevance, the toxicity of acrolein on mouse hepatocytes produced by incubation of cells with allyl alcohol was determined.

In in vitro cell systems, the toxicity of allyl alcohol can be neatly separated into “adduction” and “postadduction” phases. Mouse hepatocytes were briefly exposed to allyl alcohol, allowing formation of acrolein and protein adduction to occur. Then, prior to the cells manifesting cell membrane leakiness (ie. LDH leakage), the media was changed and the monolayers washed with phosphate-buffered saline. The cells were then layered with fresh media containing a range of hydralazine concentrations (5-50μM). Any effects of the drug in this stage will be due to “reversal” actions rather than trapping of free aldehyde. The onset of cell death was followed via the leakage of lactate dehydrogenase (LDH) into the media.

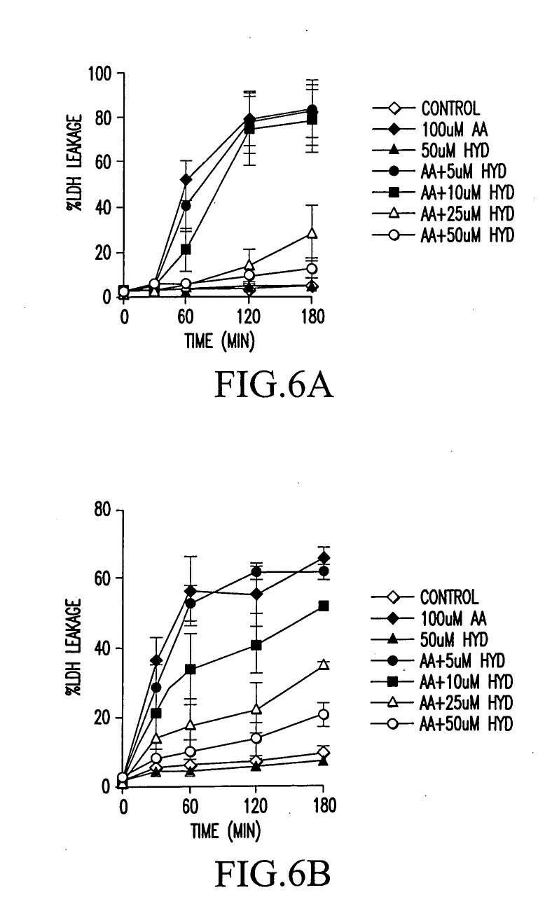

The results obtained are shown in FIG. 6. Each data point represents the mean±S.E. of 3 independent observations. Hydralazine was found to be just just as protective when added only during the secondary “postadduction phase” (Panel B) as when it was included during the entire phase of allyl alcohol toxicity (Panel A). These findings are consistent with hydralazine's ability to break bonds involved in the adduction by acrolein, and indicated that these properties are retained at low drug concentrations.

### EXAMPLE 9

Hydralazine and Dihydralazine Display Concentration-dependent Cytoprotective Potency Against the Toxicity of Smoke Extracts in Mouse Hepatocytes.

Smoke was generated by heating high-grade pine wood shavings (10 g) in a pyrex combustion chamber using a Bunsen burner as the heat source. The pine wood shavings were air-dried in a drying cabinet for 24 to 48 hours prior to use. Air flow was maintained via an inlet tube attached to a compressed air cyclinder. Smoke exiting from the chamber was passed through a water-cooled condenser and bubbled through a bubble trap, containing 20 mL phosphate-bufferred chilled in an ice bath. Combustion was allowed to proceed until completion, which typically occurred within 15 to 25 mins.

To quantify the amount of acrolein trapped in the saline solution, a UV spectrophotometric method using m-aminophenol was used. In the presence of acid and at elevated temperature (100° C.), acrolein and m-aminophenol react to form 7-hydroxyquinoline as shown in the scheme below. m-aminophenol is highly fluorescent and has strong UV-absorption properties (UVₘₐₓ used was 346 nm).

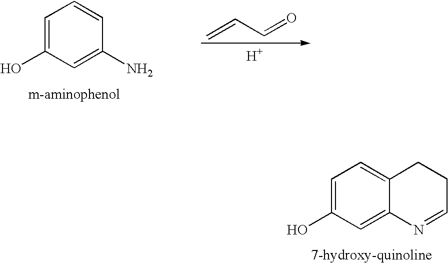

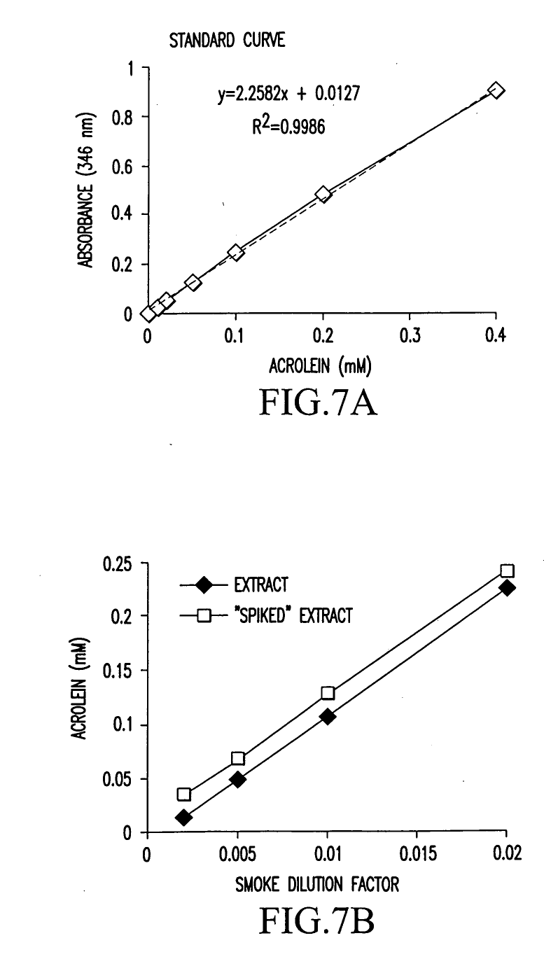

For each experiment, a new standard curve was generated, using standards containing 0.01 to 0.4 mM acrolein. A representative standard curve obtained with the assay is shown in Panel A of FIG. 7.

The assay was found to be highly linear with respect to acrolein concentration. To determine acrolein levels in freshly prepared smoke, saline smoke extracts were diluted 1/500, 1/200, 1/100 and 1/50 and 1 mL aliquots were assayed using the m-aminophenol method. To assess whether other smoke carbonyls might interfere in the assay, each of the various dilutions of smoke were spiked with 20 nmol/mL acrolein before they were carried through the acrolein assay. The results shown in Panel B of Figure demonstrate a highly linear relationship between acrolein levels and dilution factors in both spiked and untreated extracts (each point is the mean±S.E. of triplicate determinations). The assay thus provides accurate estimates of the acrolein content of smoke extracts.

Using this method, the average acrolein concentrations in 6 independent smoke extract preparations prepared under identical conditions was 18.0±2.1 mM (mean±S.E.).

To determine whether hydralazine and dihydralazine interfere with the toxicity of smoke constituents, freshly isolated mouse hepatocyte monolayers were exposed to smoke extract such that an acrolein equivalent concentration (SDAE: smoke-derived acrolein equivalents) of 50 μM was achieved in culture media (RPM11640 media). Hydralazine (HYD) and dihydralazine (DIH) were added to give final concentrations of 25, 50 and 100 μM. Cells were returned to the incubator and samples were taken for lactate dehydrogenase (LDH) determination at 60, 120 and 180 mins.

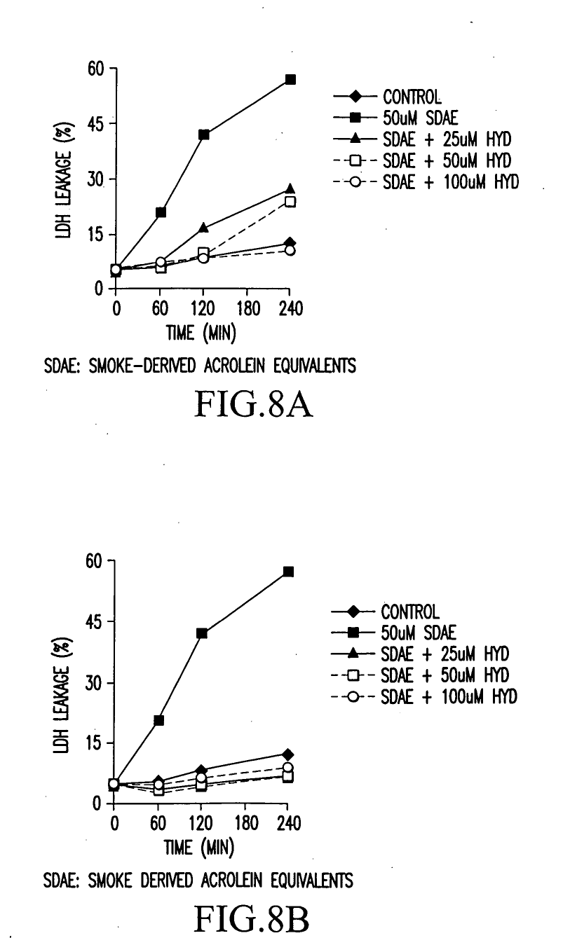

The results of a typical experiment are shown in FIG. 8. Both drugs strongly attenuated the toxicity of the smoke extract, with dihydralazine completely attenuating LDH leakage at all concentrations examined. It was also found that that the maximum concentration of both drugs (100 μM) was nontoxic to the cells over the duration of the experiments.

### EXAMPLE 10

Hydralazine Administration Results in Dose-dependent Protection Against Acrolein-mediated Hepatotoxicity in Intact Mice.

Allyl alcohol was administered to adult male Swiss mice (4-5 weeks old) as a prepared freshly solution in isotonic saline. A dose of 100 mg/kg was administered in an injection volume of approx. 0.2 mL per animal via an i.p. injection. The mice then mmediately received an i.p. injection of hydralazine to give doses of 100, 200, or 300 μmol/kg. After 4 hours mice were anaesthetised with phenobarbital and cardiac blood samples were collected. The samples were centrifuged to obtain plasma and then stored frozen at −20° C. until enzyme analyses were performed. The plasma activity of sorbitol dehydrogenase (SDH) was determined via a UV spectrophotometric procedure using fructose and NADH as substrate and cofactor, respectively. The activity of the liver marker enzyme sorbitol dehydrogenase in plasma is a marker of liver injury.

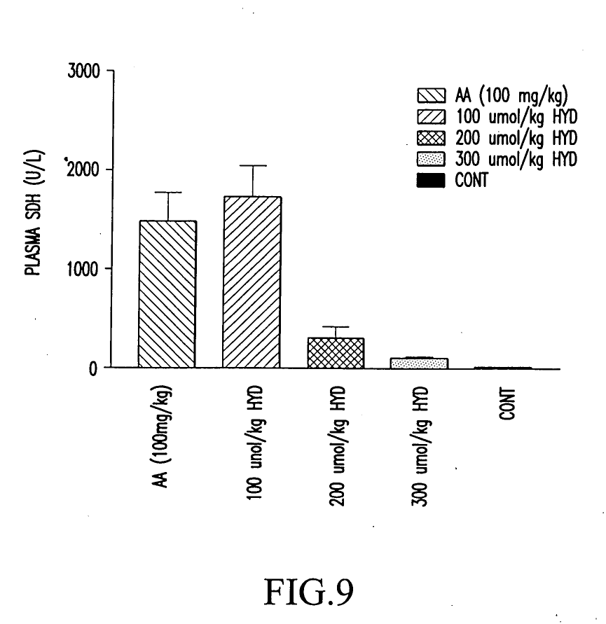

The results obtained from a representative experiment are shown in FIG. 9. Each data point represents the mean±S.E. of the following numbers of surviving mice: control group, 4; AA-only (4 mice); AA+100 μmol/kg HYD, 4; AA+200 μmol/kg HYD, 3; AA+300 μmol/kg HYD, 2.

The data is shown in FIG. 9. Each point represents the mean±S.E. of the numbers of surviving mice. As expected, allyl alcohol alone caused a strong increase in the activity of SDH in mouse plasma within 4 hrs. Co-administration of hydralazine at the lowest dose studied (100 μmol/kg) did not alter the levels of SDH. However, the highest two doses of hydralazine strongly protected against liver injury, diminishing SDH activities by 75 to 90%. These findings confirm that the ability of hydralazine to attenuate acrolein-mediated cell injury is relevant in the in vivo setting.

### EXAMPLE 11

Dihydralazine Protects Against the Cytotoxic Effects of Allylamine in Rat Neuronal Cells

Rat phaeochromocytoma (PC-12) cells were were plated at 50,000 cells per well on polylysine coated 96-well plates in DMEM media (supplemented with 10% horse serum, 5% fetal calf serum, 1 mM glutamine, nonessential amino acids and streptomycin/penicillin).

Allylamine undergoes amine oxidase-catalysed oxidation to acrolein in PC-12 cells. Allylamine and/or dihydralazine were added in 10 μL volumes to each well and the plates then placed in a 5% CO₂ incubator at 37° C. for 24 hrs. After this time, the viability of the cells was assessed using a MTT reduction assay (3-(4,5-Dimethylthiazol-2-yl)-2,5-diphenyltetrazolium bromide). Measurements were performed using a Galaxy Polar-Star multiplate reader.

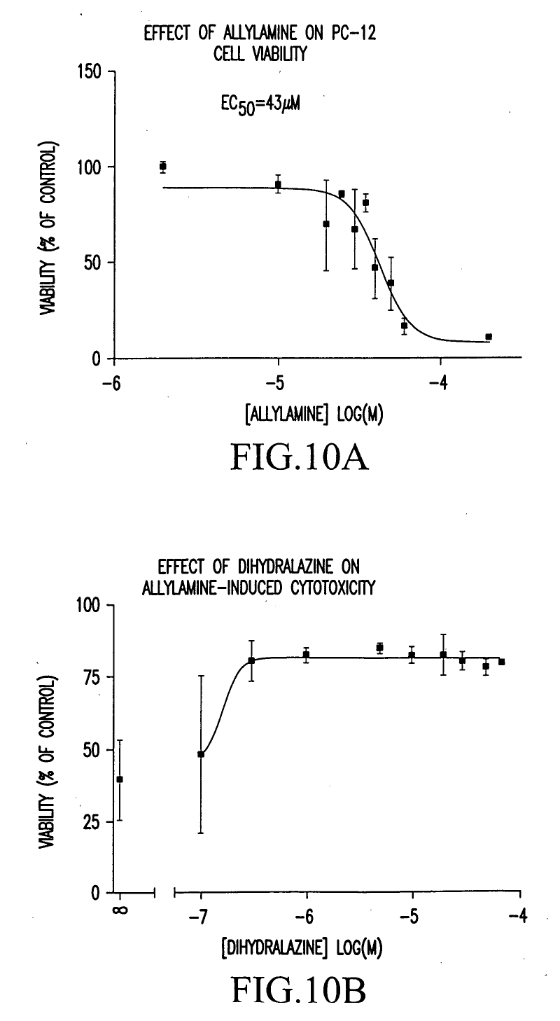

The results obtained are shown in FIG. 10, panels A and B. In each experiment, each treatment was performed with 8 replicates. The data in Panels A and B represents 3 and 2 independent experiments, respectively.

Panel A shows the concentration-response curve for allylamine cytotoxicity in PC12 cells. Exposure to 2 to 200 μM concentrations of allylamine for 24 hrs caused concentration-dependent cell death, with 50% cell death produced by approx. 45 μM allylamine. In Panel B, the effect of various concentrations of dihydralazine (0.1 to 100 μM) on the toxicity produced by concurrent exposure to 45 μM allylamine is shown. Although viability was reduced to about 40% of controls by allylamine treatment alone, concentrations of approximately 1 μM dihydralazine and higher restored cell viability to approximately 80%.

These findings are consistent with the cytoprotective properties of dihydralazine seen in the hepatocyte model.

### EXAMPLE 12

Hydralazine Displays Clear Cytoprotective Potency Against Cyclophosphamide Toxicity in Mouse Hepatocytes.

To establish that mouse hepatocytes are a suitable model for examining the toxicity of oxidative metabolites formed from cyclophosphamide, the ability of the CYP450 inhibitor proadifen to ameliorate toxicity due to cyclophosphamide exposure was first determined.

For these experiments, freshly isolated mouse hepatocytes were plated onto collagen-precoated 24-well culture plates. After a 2 to 3 hr attachment period, each well was gently washed with phosphate-buffered saline to remove nonadherent cells. RPMI1640 culture media (0.5 ml) was added to each well. In selected wells, cyclophosphamide was added to give final a concentration ranging from 100 to 1500 μM. Hydralazine and dihydralazine were added to selected wells that received 250 μM cyclophosphamide to give final hydrazinophthalazine concentrations ranging from 10 to 100 μM. Proadifen (SKF-525A) was also added to selected wells to give a final concentration of 50 μM. Hydralazine, dihydralazine, proadifen (SKF-525A) and cyclophosphamide were all dissolved directly in the culture medium without the use of organic solvents. Plates were returned to the 37° C. incubator overnight (18 hrs). The leakage of lactate dehydrogenase (LDH) into the culture media was assessed as an indicator of cell death the following day.

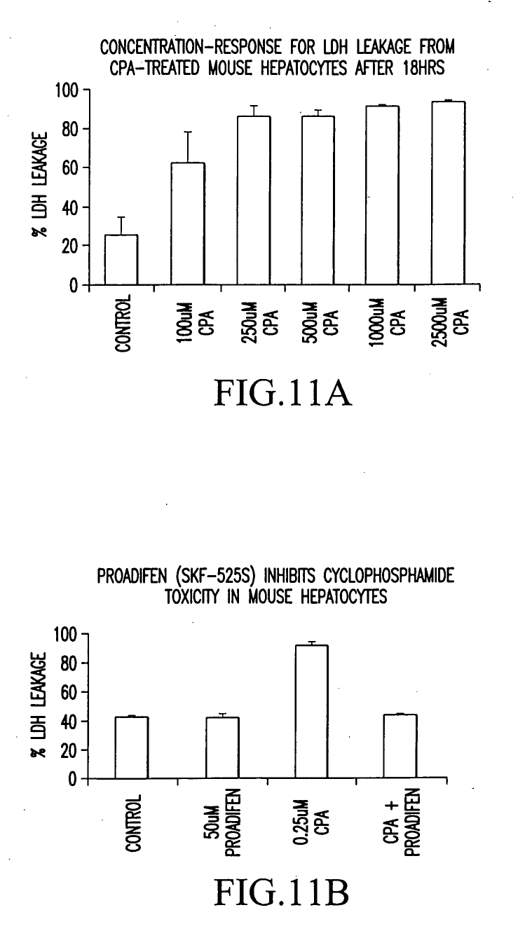

The concentration-response for LDH leakage from cells treated with a range of cyclophosphamide concentrations after an overnight incubation is shown in Panel A of FIG. 11. The data shown is the mean±standard error of three determinations.

The data shows that cyclophosphamide was toxic to the cells, with concentrations of 250 μM and greater producing complete cell death. The effect of the CYP450 inhibitor proadifen on the toxicity of 250 μM cyclophosphamide after an overnight exposure is shown in Panel B. This CYP450 inhibitor abolished the cyclophosphamide-induced increase in LDH leakage, indicating that a CYP-derived oxidation product mediated the toxicity of cyclophosphamide in this model.

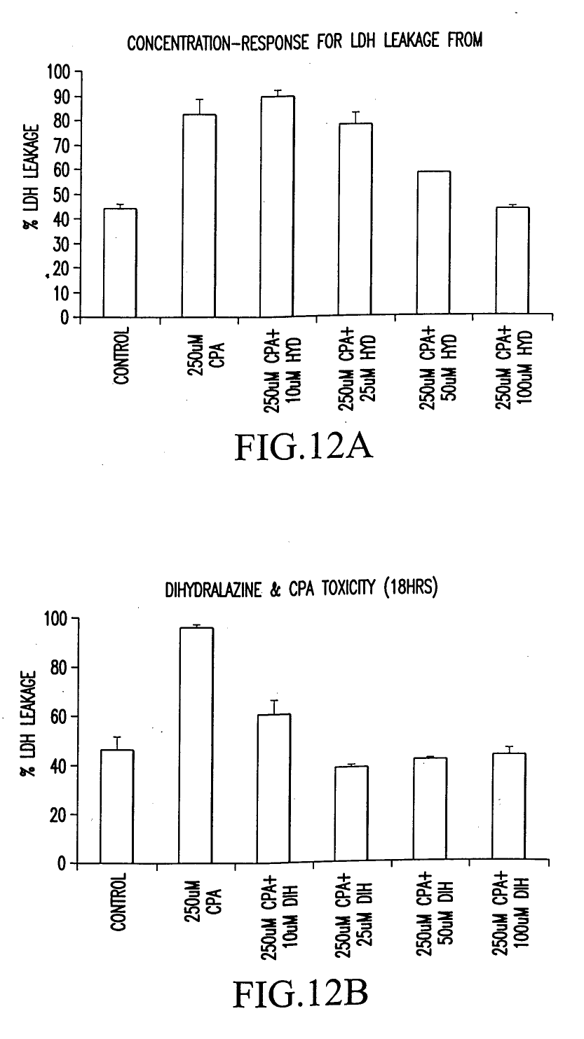

The effect of hydralazine and dihydralazine on cyclophosphamide toxicity in mouse hepatocytes was examined using a single toxic concentration of the anticancer drug (250 μM). FIG. 12 (mean±standard error of three determinations) shows that both hydralazine (Panel A) and dihydralazine (Panel B) attenuated the toxicity of cyclophosphamide during an overnight incubation. These findings are consistent with the ability of these hydrazino drugs to reverse cell and protein damage caused by acrolein, formed via rearrangement and fragmentation of the CYP450 oxidation product of the drug.

### EXAMPLE 13

Loss of Acrolein-lysine Adducts in Mouse Hepatocytes Accompanies Protection Against Acute Toxicity by Hydralazine

(i) Cell Culture Experiments: Hepatocytes were prepared by collagenase digestion of mouse livers as outlined previously (Burcham and Fontaine, 2001). Cells were washed four times in collagenase-free Krebs-Henseleit buffer before they were resuspended at a density of 1×10⁶ cells per ml in RPMI 1640 media supplemented with 0.2% BSA, 0.03% L-glutamine, penicillin (50 U/ml) and streptomycin (50 μg/ml). Hepatocytes were then layered on collagen-coated 60-mm polystyrene dishes before they were placed in a humidified CO₂ incubator at 37° C. (5% CO₂). After 2 h, non-adherent cells were removed by washing the dishes twice with phosphate-buffered saline (PBS) before fresh aliquots of RPMI 1640 (not supplemented with BSA) were added. Hydralazine and were added to media immediately before use. For the experiments involving direct addition of acrolein to cell media, Krebs-Henseleit buffer (pH 7.3) supplemented with glucose (5 g/L) and pyruvate (1 mM) was used in place of RPMI1640, in an effort to minimize extracellular side-reactions between free acrolein and nucleophilic buffer constituents (Horton et al., 1999). Dishes were returned to the incubator and samples of culture media were taken for the assessment of lactate dehydrogenase (LDH) leakage at 0, 30, 60, 120, and 180 min or as indicated. LDH activity was measured using a spectrophotometric procedure as described in Burcham and Fontaine (2001) J. Biochem Molec. Toxicol. 15:309-316. For the determination of total LDH activity, cells were lysed by adding 250 μL of 5% Triton X-100 to each dish before they were sonicated for 15 seconds using a Labsonic 1510B Cell Disrupter (Braun Melsungen AG, Germany).

(ii) SDS/PAGE and Western Blotting: Cell monolayers were rinsed with PBS then resuspended in 0.3 ml cold lysis buffer (contained 0.25% SDS, 30% glycerol, 50 mM Tris-HCI [pH 6.8], 0.5% Triton X-100, Sigma Protease Inhibitor cocktail [5 μl/ml], 0.2 mg/ml PMSF and 1 mM benzamidine). In experiments where cell proteins were assessed for acrolein adducts, 50 mM phosphate buffer (pH 7.0) was substituted for Tris-HCl. Lysates were prepared by sonicating the samples (60 sec) on ice and then centrifuging the resulting suspensions at 5,000×g for 10 min at 4° C. Following protein estimation (Pierce BCA Kit), 50 μg protein was resolved overnight at 4 V/cm on either a 4-20% gradient acrylamide gel (Jule Inc Biotechnologies, Milford, Conn., USA) or a 10% acrylamide gel. After transfer to nitrocellulose (100 V, 30 min), membranes were blocked with 5% nonfat milk in PBS and then reacted for 60 min with 1/1000 dilutions of respective rabbit antiserum (raised against either acrolein-modified KLH. Following washing and exposure of membranes to horseradish peroxidase-coupled goat anti-rabbit IgG serum (Pierce Immunopure, 1/10,000 dilution, 30 min), membranes were washed and developed using Pierce Super Signal West Pico chemiluminescence reagent and Kodak BioMax Light film and the resulting images were analysed via densitometry using Kodak Digital Science software.

(iii) Statistical Analysis: Cell toxicity data (i.e. LDH leakage timecourse curves) were analysed via 2-way ANOVA followed by Bonferroni's post test using GraphPad Prism 4.01 for Windows software.

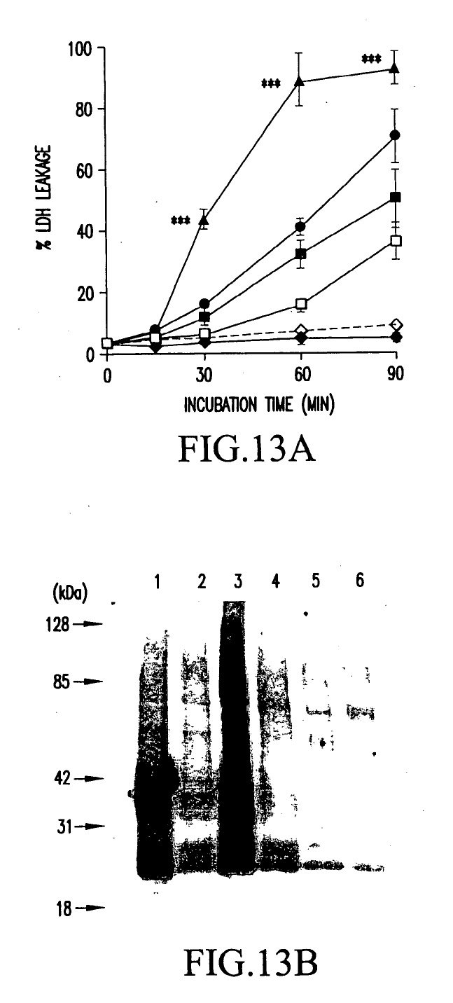

(iv) Results: To determine whether hydralazine protects cell proteins against acrolein adduction, we used Western blotting to detect acrolein-lysine adducts in mouse hepatocyte proteins after a 15 min exposure to a cytotoxic concentration of acrolein. The experiment was performed in amino acid-free buffer, thereby minimizing side-reactions involving acrolein. The antibody used to detect adducted proteins was the antibody raised against acrolein-modified KLH as described in Example 4, which is highly selective for acrolein-adducted lysine residues. Due to high basal levels of adducts in controls and concerns over adduct stability during SDS/PAGE, a high concentration of acrolein (0.5 mM) was used in this experiment, necessitating high concentrations of hydralazine to afford cytoprotection. The latter was confirmed by following the time-course of lactate dehydrogenase (LDH) leakage into culture media (FIG. 13A). Protein adducts were assessed after a 15 min exposure period to avoid any loss of adducted proteins via a ruptured cell membrane that was evident at latter time points (FIG. 13B). Compared to levels in control cells (Lane 1, FIG. 13B), exposure to 0.5 mM acrolein increased adduction for a number of proteins in the 20 to 85 kDa range (Lane 3, FIG. 13B). Hydralazine had a striking effect on the immunoreactivity of proteins in both control (Lane 2) and acrolein-exposed cells (Lanes 4 to 6, FIG. 13B), with the drug decreasing levels of adducted proteins below control values. This finding implied that the drug did not simply prevent protein adduction by free acrolein, but instead interfered with the immunoreactivity of acrolein-adducted proteins.

### EXAMPLE 14

Electrospray Ionization-mass Spectrometry (ESI-MS) Spectra Obtained During Analysis of Acrolein- and Hydralazine-modified Preproenkephalin Fragment

(i) Mass Spectrometry Experiments: Electrospray-MS was used to detect drug-trapped adducts in a model peptide, preproenkephalin fragment 128-140 (PPE). PPE is a 13-mer peptide that possesses a single central lysine residue (GGEVLGKRYGGFM, MW 1370). PPE and acrolein were dissolved in H₂O to give final concentrations of 100 and 1000 μM, respectively. After 30 min at 37° C., an equivalent volume of hydralazine solution was added to give a final drug concentration that was 10-fold in excess relative to acrolein. The samples were returned to the incubator for a further 30 min. Immediately prior to injection into the MS, samples were diluted 1:1 with an aqueous solution comprising 2% glacial acetic acid and 50% acetonitrile. MS analyses were performed using a Finnigan LCQ mass spectrometer in positive ESI mode (Finnigan, San Jose, Calif.). Samples were introduced into the electrospray source using a syringe pump at a flow rate of 8 μL/min. The spray voltage was set at 4.8 kV with a capillary temperature of 200° C. and a cylinder gas (nitrogen) pressure of 100 psi. Mass spectra were collected by scanning a m/z range of 1000 to 2000.

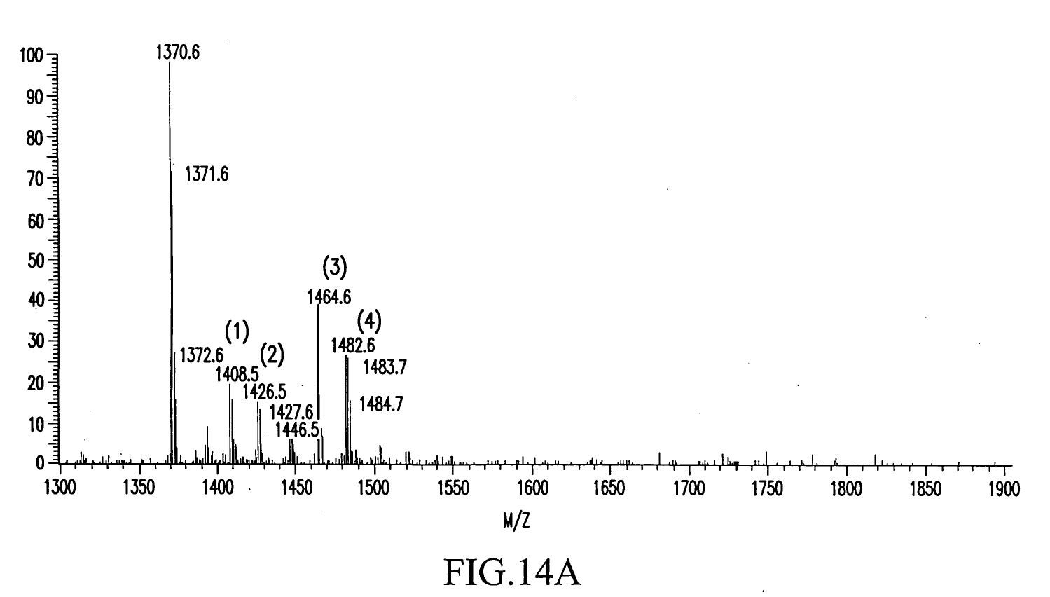

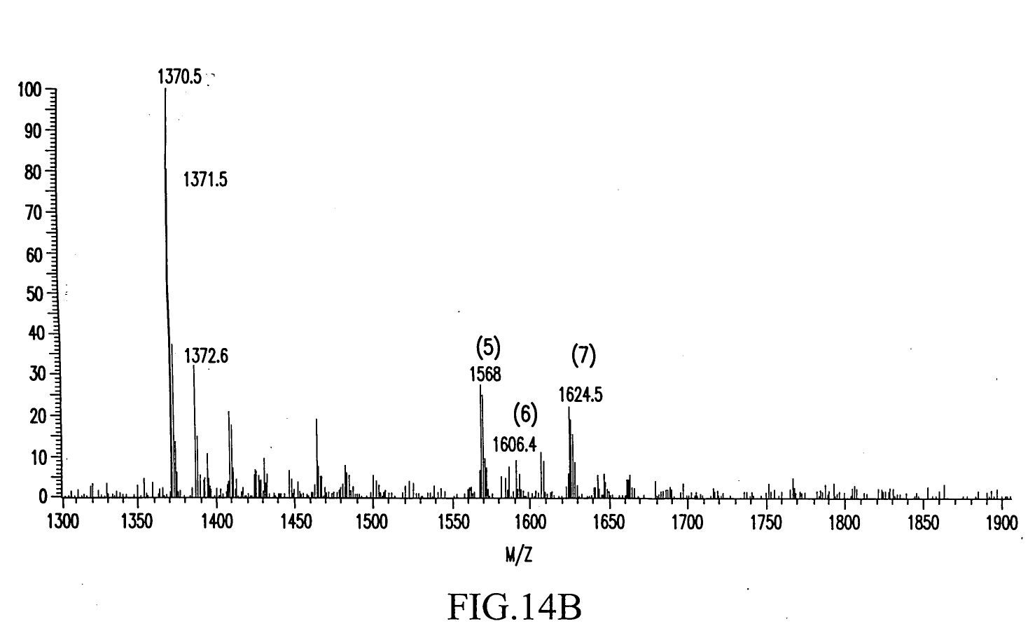

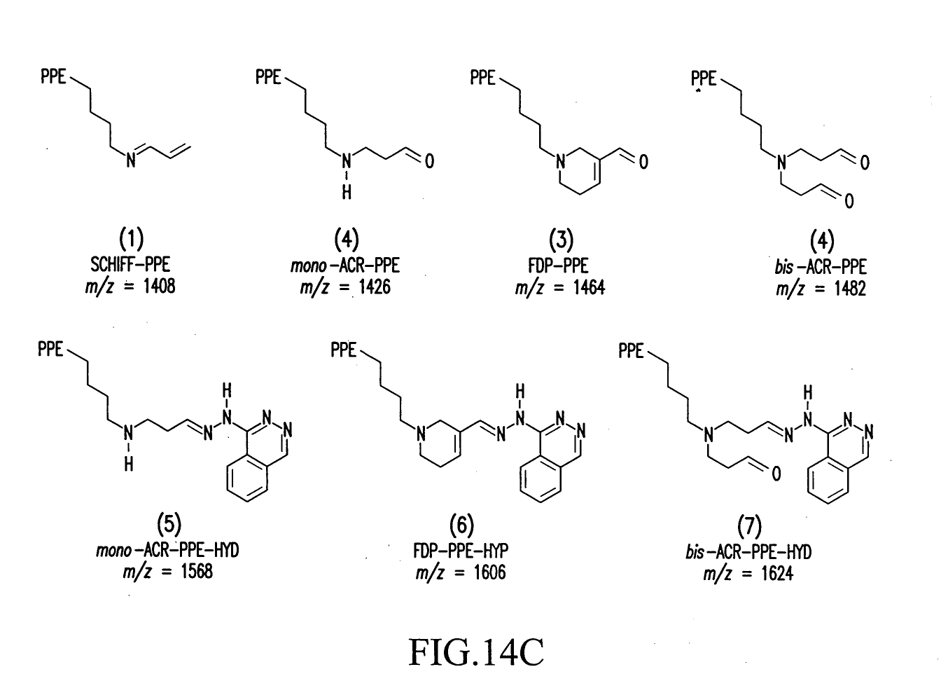

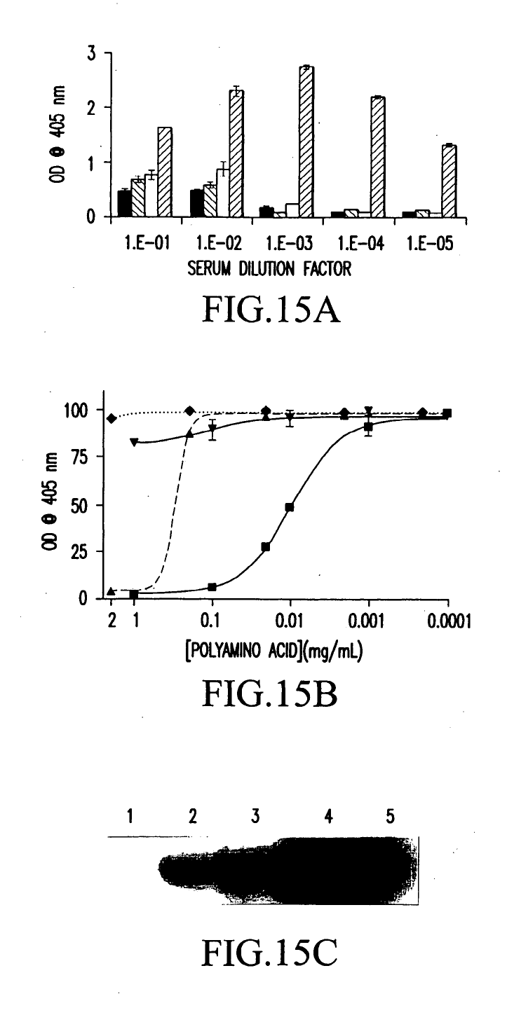

(ii) Results: We used electrospray ionization-mass spectrometry (ESI-MS) to monitor reaction products during modification of a lysine-containing model peptide by acrolein, and also identify species formed upon addition of hydralazine. The peptide used, preproenkephalin fragment 128-140 (PPE), was selected because it possesses a single central lysine, which has been shown to be a key target during reactions of acrolein with proteins. Furthermore, PPE's suitability for use in this study was enhanced by the fact that it lacks other residues known to react with acrolein (e.g. cysteine, histidine). For these experiments, PPE (100 μM in H₂O) was reacted with a 10-fold excess of acrolein for 30 min (37° C.). The mixtures were then divided into 2 portions before they were subjected to a second 30 min reaction in the presence and absence of a 10-fold molar excess of hydralazine (c.f. acrolein). Finally, the respective samples were analysed via ESI-MS, with representative MS spectra shown in FIG. 14, which also shows the structures of the main species detected during these analyses. Analysis of native PPE revealed a dominant MH⁺ ion corresponding to unmodified peptide at m/z 1370 (data not shown). The spectrum obtained during MS analysis of peptide that had been treated with hydralazine alone was essentially identical to that obtained from unmodified PPE (data not shown). In FIG. 14A, acrolein's diverse reactivity with lysine is evident: a minor MH⁺ ion corresponding to a Schiff base product formed during reaction of acrolein's carbonyl group with PPE is evident at m/z 1408 (1), while the expected MH⁺ ions for the mono- and bis-Michael adducts are detected at m/z 1426 (2) and 1482 (4) (FIG. 14A). An MH⁺ ion corresponding to formation of the condensed-ring product FDP-lysine in PPE is evident at m/z 1464 (3) (FIG. 14A). Addition of hydralazine to acrolein-modified PPE generated several new ions (FIG. 14B). The relative abundance of the mono adduct (m/z 1426) appeared to be diminished, with the expected hydrazone reaction product evident at m/z 1568 (5). A single hydralazine molecule also reacted with the bis-acrolein adduct, generating a hydrazone at m/z 1624 (7) (FIG. 14B). Finally, an ion corresponding to reaction of hydralazine with the formyl group of FDP-lysine is detected at m/z 1606 (6). Due to inherent limitations of ESI-MS analysis, strict quantitative conclusions cannot be drawn from the data in FIG. 14B, but the findings clearly confirm formation of hydrazone species during adduct-trapping reactions by hydralazine at carbonyl-retaining acrolein adducts in PPE.

### EXAMPLE 15

Immunochemical Detection of Hydralazine-trapped Acrolein Adducts in BSA

(i) Antibody Production and Characterization: To prepare antiserum against hydralazine-stabilised acrolein-adducted protein, keyhole limpet hemocyanin (KLH, 5 mg/ml) was modified with 5 mM acrolein for 25 min then hydralazine was added to a final concentration of 10 mM. After 4 h at 37° C., the mixture was diluted 1:9 with PBS, then 3:1 with Freunds Complete Adjuvant before it was administered to an adult male NZ white rabbit (1 mg over 10 injection sites). The rabbit received 2 subsequent booster injections at 3-week intervals using freshly prepared antigen diluted in Freunds Incomplete Adjuvant. Ten days after the final boost, the rabbit was anaesthetized and whole blood was collected via cardiac puncture. Serum was prepared and analysed for crossreactivity against unmodified BSA or BSA that had been modified by acrolein in the presence and absence of hydralazine using a competitive ELISA similar to that described in Burcham et al., (2003) Chem. Res. Toxicol 16: 1196-1201.

Epitope characterization was carried out using acrolein/hydralazine-adducted poly-L-lysine and poly-L-histidine in antigen competition ELISA experiments as described in Chen et al. (1992) Biocehm J. 288:249-254. To make the inhibitors, aminoacyl polymers were dissolved in 50 mM sodium phosphate buffer (pH 7.0) to a final concentration of 1 mg/ml, then acrolein was added to give a 1:1 aldehyde:monomer molar ratio. Reactions were allowed to proceed for 30 min at 37° C., then hydralazine was added to give a 2:1 molar ratio relative to acrolein. After reaction overnight at 37° C., the inhibitors were stored at −20° C. until use in competitive ELISA experiments as described in Burcham et al. (2003) Chem. Res. Toxicol 16: 1196-1201.

Results: The previous findings suggested the possibility of raising antibodies against “hydralazine-trapped” acrolein-protein adducts, in the expectation that such a tool would be very useful for exploring the biological significance of hydralazine's reactivity with acrolein-adducted proteins. We thus immunized a rabbit with antigen that had been prepared by sequentially modifying KLH with acrolein and hydralazine. Testing of the resulting antiserum in an ELISA procedure revealed that it is highly reactive toward BSA that was modified by acrolein and hydralazine, but not toward unmodified BSA or BSA that was modified by acrolein or hydralazine alone (FIG. 15A). Furthermore, during competitive ELISA studies using acrolein and hydralazine-modified polyaminoacids as immunoinhibitors, it was established that the antiserum strongly recognized hydralazine-acrolein-lysine species, with lesser activity at hydralazine-acrolein-histidine complexes (FIG. 15B). Using the antiserum, we then verified that “adduct-trapping” accompanied abolition of acrolein-lysine adducts by hydralazine under the conditions used in FIG. 13B. Thus BSA was exposed to 1 mM acrolein for 30 min, then to 50 to 500 μM concentrations of hydralazine for 30 min. The Western blot shown in FIG. 15C reveals strong adduct-trapping by hydralazine over the concentration range that abolished acrolein-lysine adducts in FIG. 13B.

### EXAMPLE 16

Adduct-trapping Accompanies Cytoprotection Against Acrolein-mediated Toxicity by Hydralazine

(i) Cell Culture: RPMI1640 was used in experiments that involved the use of allyl alcohol as an intracellular acrolein precursor. Dishes were returned to the incubator and samples of culture media were taken for the assessment of lactate dehydrogenase (LDH) leakage at 0, 30, 60, 120, and 180 min or as indicated. In experiments where hepatocytes were exposed to a toxic. concentration of allyl alcohol (100 μM) for 25 min, the culture media was removed and the monolayers were washed once with PBS. The cells were then layered with fresh solutions of culture media, including media containing 5 to 50 μM hydralazine. The cells were returned to the incubator and aliquots of media were removed for the determination of LDH activity.

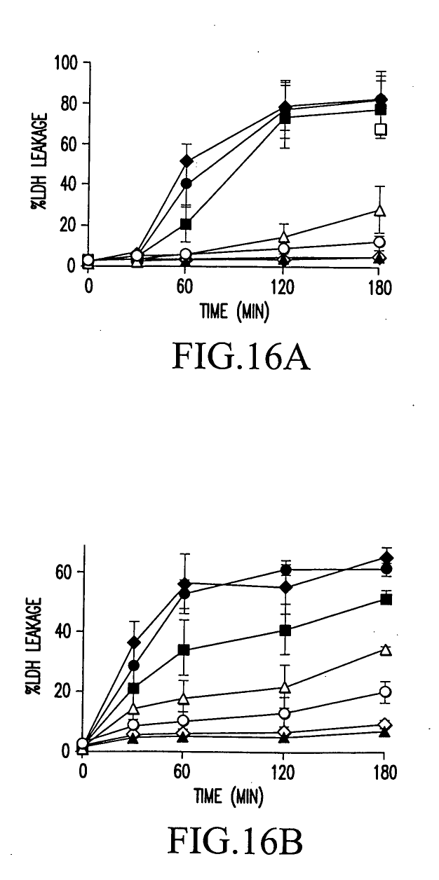

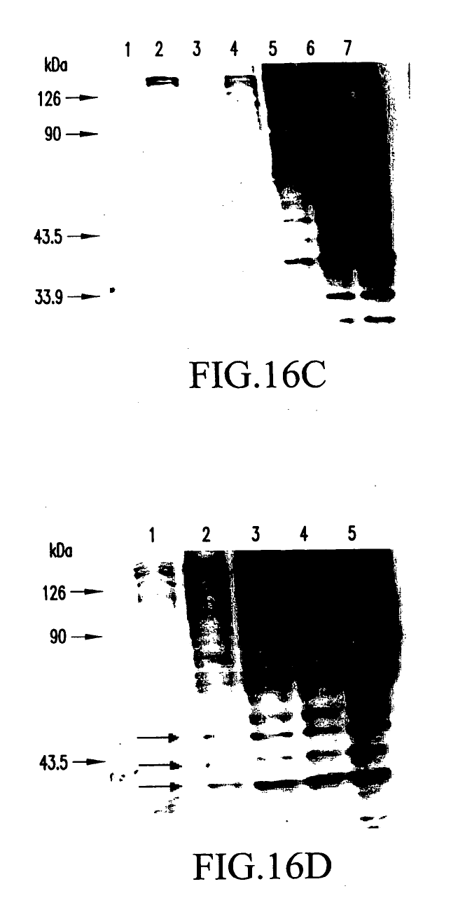

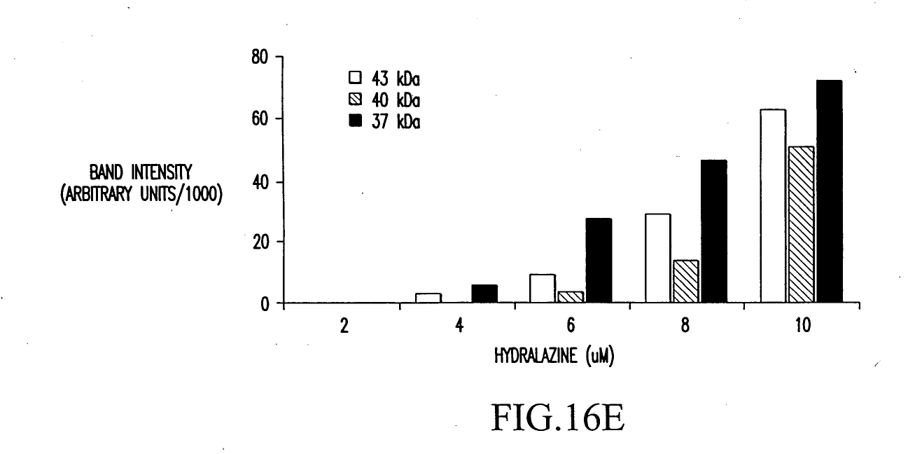

(ii) Results: To determine if hydralazine's adduct-trapping actions accompany the protection it affords against acrolein-mediated toxicity, we conducted experiments in mouse hepatocytes using the acrolein precursor allyl alcohol, which causes time- and concentration-dependent toxicity in these cells via alcohol dehydrogenase-catalysed conversion to acrolein. We hypothesized that if protein adduct-trapping played any role in cytoprotection, hydralazine should protect cells when present only during the secondary “postadduction” phase. To assess this possibility, mouse liver cells were treated briefly with allyl alcohol (100 μM) to allow metabolism and protein adduction to occur. Prior to the onset of cell death (25 min) the culture media was removed and the cells were washed with PBS. They were then exposed to fresh media containing 5 to 50 μM hydralazine for up to 3 h, over which time the onset of cell death was assessed at regular intervals via measurements of LDH leakage. Consistent with the hypothesis that cytoprotection involves an ability to interfere with events that are “downstream” of metabolism and adduction, FIG. 16A shows hydralazine was essentially as protective during the secondary “postadduction phase” as when it was present during the entire phase of toxicity (FIG. 16B).

Western blotting was then used to determine whether adduct-trapping occured under conditions in which cytoprotection is afforded by hydralazine during the “postadduction phase” of allyl alcohol toxicity. FIG. 16C shows drug-trapped adducts in proteins from allyl alcohol-pretreated cells after a secondary 30 min incubation in the presence and absence of hydralazine. No immunoreactivity was evident in controls (Lane 1, FIG. 16C) or cells pretreated only with allyl alcohol (Lane 3), although trapped adducts were detected in two≈130 kDa proteins in control cells exposed only to hydralazine (50 μM), presumably reflecting reactions of hydralazine with carbonyl-retaining protein adducts of endogenous origin (Lane 2). In striking contrast, intense, concentration-dependent adduct-trapping occurred in allyl alcohol-pretreated cells that were subjected to a second incubation with a range of cytoprotective hydralazine concentrations (5 to 50 μM, Lanes 4 to 6). For the data shown in FIG. 16D, a greater amount of protein was analyzed to determine whether adduct-trapping also occurred in the “postadduction phase” at low hydralazine concentrations (2 to 10 μM) that are of greater relevance to human pharmacology. Clear concentration-dependent trapping was evident over this range, with densitometric analysis of several mid-sized proteins (37, 40 and 43 kDa, indicated with arrows in Panel E) revealing 12- to-200-fold increases in band intensity over the drug concentration range of 4 of 10 μM (FIG. 16E). Faint adduct-trapping reactions involving the ≈130-kDa proteins are evident even at the lowest hydralazine concentration studied (2 μM, Lane 1). This concentration is close to peak plasma levels of hydralazine in hypertensive human subjects after a 50 mg oral dose of hydralazine.

### EXAMPLE 17

Hydralazine Affords Dose-Dependent Protection Against Allyl Alcohol Hepatotoxicity in Mice

(i) Animal treatments: To diminish inter-animal variability in hepatic responsiveness to allyl alcohol, food was withheld for 15 hours prior to commencing experiments. On the day of experimentation, mice received allyl alcohol (60-100 mg/kg [approx. 1100-1800 μmol/kg]) either alone or in conjunction with hydralazine (100-300 μmol/kg) via a single intraperitoneal injection (the dosing volume was 10 mL/kg). Control mice received either vehicle only (phosphate buffered saline [PBS], 50 mM, pH 7.4) or 300 μmol/kg hydralazine. In a related experiment, the time dependence of hydralazine-induced hepatoprotection was explored, with 200 μmol/kg hydralazine administered to mice either 0, 20 or 30 minutes after a single 90 mg/kg dose of allyl alcohol. Four hours after hydralazine administration, mice were anaesthetized with pentobarbitone (6 mg/animal, i.p.) and blood was collected via open cardiac puncture. Plasma was prepared and stored at −20° C. until use. After perfusion with 25% sucrose, the right medial lobe was removed for use in immunohistochemical studies or Western blotting procedures. The remaining tissue portions were homogenized in 9 volumes of cold 3% perchloric acid and then centrifuged at 7,000×g for 5 minutes. The resulting supernatant was used for GSH determination as outlined below.

(ii) Biochemical Analyses: Plasma activities of sorbitol dehydrogenase (SDH) and glutamate pyruvate transaminase (GPT) were measured to assess the severity of liver injury. For SDH determination, 0.1 mL plasma was diluted with 0.5 mL Tris-HCl buffer (0.1 M, pH 7.5) containing 0.4 mM NADH. After a 10 min incubation at room temperature to allow removal of interfering metabolites, reactions were started by adding 0.1 mL fructose solution (4.0 M prepared in the abovementioned Tris-HCl buffer). NADH oxidation was then followed for 3 minutes at 340 nm using a Metertek SP-830 spectrophotometer (Analytical Equipment Co., Adelaide, South Australia). SDH activity was then expressed as Units/L, where 1 Unit is the activity producing 1 mol of NAD⁺ per minute at 25° C. For the estimation of GPT activity, a 2-step reaction was used where pyruvate, the product of GPT-catalyzed alanine deamination, was reduced to lactate in a NADH-dependent reaction catalysed by lactate dehydrogenase. Briefly, 0.1 mL plasma was added to a 0.6 mL reaction mixture that comprised 1.0 M alanine and 10 Units/mL lactate dehydrogenase (Sigma, Type II, rabbit muscle) prepared in potassium phosphate buffer (0.1 M, pH 7.4). A 10 μL volume of stock NADH solution (13 mM, prepared in 120 mM sodium bicarbonate) was then added to each sample. After mixing, the samples were allowed to stand at room temperature for 3 minutes, after which reactions were started by adding 20 μL of α-ketoglutarate solution (0.66 M). NADH oxidation was then followed for 3 minutes at 340 nm using the Metertek SP-830 spectrophotometer. To assess any possible interference by hydralazine with the enzyme assays, 0.3 mM hydralazine was added to cuvets containing aliquots of serum from allyl alcohol-treated mice. This treatment had no affect on either SDH or GPT activity (data not shown). GSH estimation was via a procedure measuring a fluorescent isoindole formed upon derivitization of GSH by o-phthaldialdehyde. Briefly, a standard curve was prepared over the range of 100 to 1000 ng GSH using 3% perchloric acid. Samples and standards were then neutralized by adding 0.16 mL of 2.5 M NaOH for each mL of perchloric acid extract. Next, 50 μL volumes of samples or standards were added to individual wells of 96-well microplates which contained 0.2 mL Tris-HCl buffer (0.2 M, pH 8.0, containing 1 mM EDTA). After the addition of 10 μL o-phthaldialdehyde (1 mg/mL stock in methanol), the reactions were allowed to proceed for 20 min in the dark. The fluorescence of each sample was then determined via a PolarStar Galaxy microplate reader (BMG Labtechnologies, Durham, N.C.) using respective excitation and emission wavelengths of 340 and 420 nm. The hepatic GSH content was then expressed as μg/mg liver.

(iii) Statistical Analysis: Serum levels of SDH and GPT in treated mice were compared to values from vehicle-treated control mice using Kruskal Wallis analysis with a Dunns test. Liver GSH levels were compared to control values via 1 way ANOVA followed by Dunnett's post hoc test.

(iv) Results: To facilitate later experiments, we conducted a pilot study to identify a dose of allyl alcohol that consistently elevated plasma SDH (sorbitol dehydrogenase) and GPT (glutamate pyruvate transaminase) activities within a 4 hour period in the mouse strain used in this study. The 4 hour duration was based on results indicating that maximal elevation of liver enzymes in plasma occurs 2 to 3 hours after allyl alcohol administration in mice (data not shown). For our pilot study, plasma SDH and GPT activities were measured 4 hours after mice received 0, 60, 80, 90 or 100 mg/kg allyl alcohol (data not shown, N=6-13). Since 90 mg/kg allyl alcohol (i.p.) elevated plasma SDH and GPT activities 30- and 24-fold over controls at this time, and had decreased hepatic GSH stores by 65%, this dose was judged suitable for use in subsequent experiments. Also, no fatalities accompanied the 90 mg/kg dose, compared to 30% mortality in mice receiving 100 mg/kg allyl alcohol (data not shown).

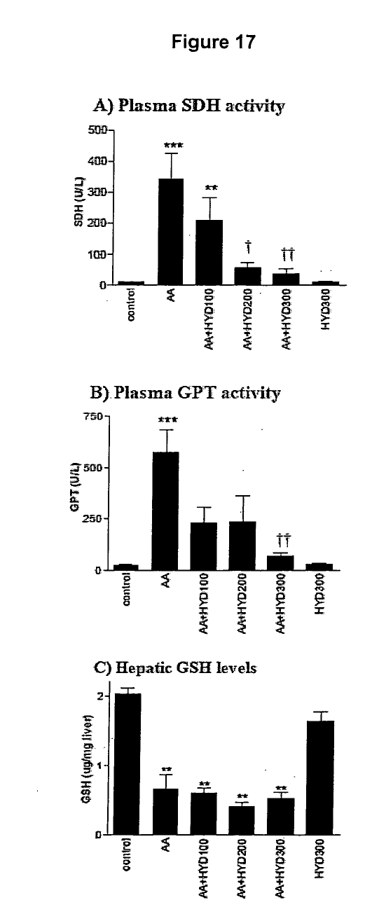

FIG. 17 indicates that hydralazine afforded clear, dose-dependent protection against allyl alcohol-induced changes in plasma enzymes in whole mice, with 300 μmol/kg hydralazine almost totally abolishing the changes in both SDH (Panel A) and GPT (Panel B) activities (p<0.01). Using the dose-response data shown in FIG. 2, hydralazine doses affording half-maximal protection against liver injury were estimated as 160 and 80 μmol/kg for SDH and GPT, respectively.

### EXAMPLE 18

Hepatoprotective Doses of Hydralazine Do Not Prevent Hepatic GSH Depletion

GSH depletion is of fundamental importance in allyl alcohol toxicity, with irreversible liver injury typically occurring after hepatic GSH is diminished below a critical threshold. Moreover, allyl alcohol hepatotoxicity is abrogated by interventions that either increase hepatic GSH or upregulate glutathione-S-transferase expression. Notwithstanding these considerations, hepatoprotective doses of hydralazine had no effect upon the hepatic GSH depletion caused by allyl alcohol (FIG. 17C). Hence the hepatic GSH content in mice that received the fully hepatoprotective dose of 300 μmol/kg hydralazine was unchanged from that in allyl alcohol-only treated mice (p>0.05).

### EXAMPLE 19

Delayed Administration of Hydralazine Abolishes the Hepatoprotection Against Allyl Alcohol Hepatotoxicity

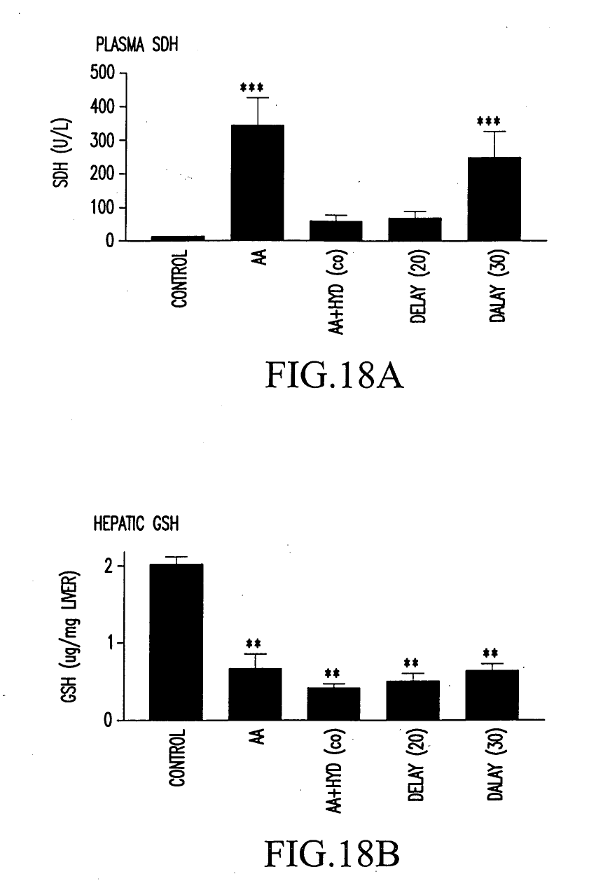

To determine whether the protective effects of hydralazine in vivo might involve comparable interference with “early” events in acrolein-mediated cell injury, we investigated the time dependence for the drug's protective actions against allyl alcohol hepatotoxicity. For this experiment, 200 μmol/kg hydralazine was administered to mice either 0, 20 or 30 minutes after they received 90 mg/kg allyl alcohol. Four hours after receiving the initial dose of allyl alcohol, animals were sacrificed for the determination of plasma enzymes and hepatic GSH. We predicted that if interference with “early” adduction chemistry underlies hepatoprotection, hydralazine would be less protective when administered 30 minutes after allyl alcohol than at earlier time points (the 30 minute period was the latest time to which drug administration could be delayed since irreversible liver damage in the form of enhanced enzyme leakage was detected at latter time points—data not shown). The results in FIG. 18 confirm that hydralazine was strongly hepatoprotective when either co-administered with allyl alcohol or following a 20 minute delay (FIG. 18A). However, if a 30 minute period was allowed between the administration of allyl alcohol and hydralazine, the drug's hepatoprotective efficacy was diminished (FIG. 18A, p<0.001 relative to saline-treated control). No differences in the degree of hepatic GSH depletion were evident between these various dosing regimens (FIG. 18B).

### EXAMPLE 20

Western Blot Analysis Reveals Intense, Dose-Dependent Adduct-Trapping by Hydralazine in Mouse Liver

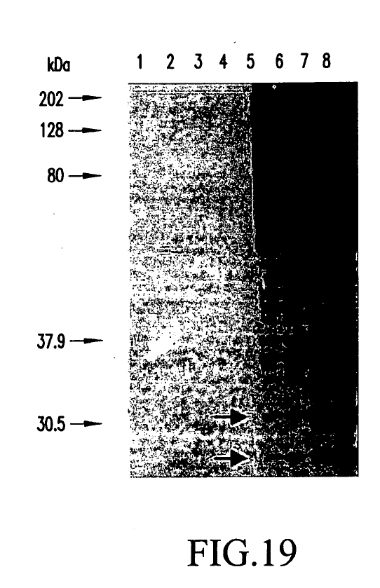

Rabbit antiserum raised against hydralazine- and acrolein-modified KLH was used to determine whether “adduct-trapping” accompanies hepatoprotection by hydralazine in allyl alcohol-treated mice. A Western blot depicting trapped adducts in proteins recovered from mouse liver 60 minutes after the administration of 90 mg/kg allyl alcohol with or without hydralazine (100 or 200 μmol/kg, i.p.) is shown in FIG. 19. Lanes 1 to 4 reveal a lack of immunoreactivity in proteins from mice treated with either injection vehicle only (Lane 1), allyl alcohol only (Lane 2), or 100 (Lane 3) or 200 μmol/kg hydralazine only (Lane 4). The lack of signals in these lanes concurs with the previous finding that the antiserum is highly specific for hydralazine/acrolein-adducted proteins. In sharp contrast, strong adduct-trapping by hydralazine was evident in the livers of two allyl alcohol-treated mice exposed to 100 μmol/kg hydralazine (Lanes 5 and 6). Some 20 to 25 proteins can be distinguished as targets for hydralazine in Lanes 5 and 6, confirming thatfacrolein generates drug-reactive adducts in a diverse range of tissue proteins. Doubling the dose of hydralazine increased the intensity of adduct-trapping in two additional animals (Lanes 7 and 8), but due to signal saturation, bands corresponding to proteins with masses greater than 40 kDa are poorly resolved (Lanes 7 and 8). In the case of 2 small well-resolved protein targets (26 and 31 kDa, depicted with arrows on FIG. 19), densitometric analysis revealed 2.6- and 2.4-fold elevations in signal intensity respectively in animals receiving 200 μmol/kg hydralazine compared to the lower dose.

### EXAMPLE 21

Adduct-Trapping Occurs Diffusely Throughout the Liver Lobule

(i) Immunohistochemical Methods: Following drug treatments, mice were anaesthetized and their livers were perfused with 25% sucrose. The right medial lobe was removed and frozen in liquid nitrogen before storage at −20° C. Liver sections (5 μm) were prepared using a cryostat maintained at −20° C. and following drying they were fixed in methocarn solution (methanol:chloroform:acetic acid, 6:3:1) for 20 minutes. Following brief rehydration in ethanol, slices were blocked in 10% skim milk/PBS for 1 hour. After treatment overnight at 4° C. with primary antibody (rabbit antiserum raised against hydralazine/acrolein-modified KLH diluted 1/750 in 10% nonfat milk/PBS), the sections were washed in PBS before they were treated with fluorescein isothiocyanate-labeled goat anti-rabbit IgG (Pierce, 1/400 in 10% nonfat milk/PBS) at room temperature for 1 hour. The slices were washed in 0.05% Tween 20 in PBS, mounted in 50% glycerol/PBS and then viewed using an Olympus BX50WI fluorescence microscope (Olympus Optical Company, Japan). In a related experiment, liver tissue was analyzed following recovery from mice 4 hours after they received a 300 μmol/kg (i.p.) dose of (1E)-acrylaldehyde 1-[1-phthalazinyl]-hydrazone, the main product formed during trapping reactions between free acrolein and hydralazine. (1E)-acrylaldehyde 1-[1-phthalazinyl]-hydrazone was synthesized from acrolein and hydralazine and its purity confirmed via NMR and mass spectrometric analysis.¹

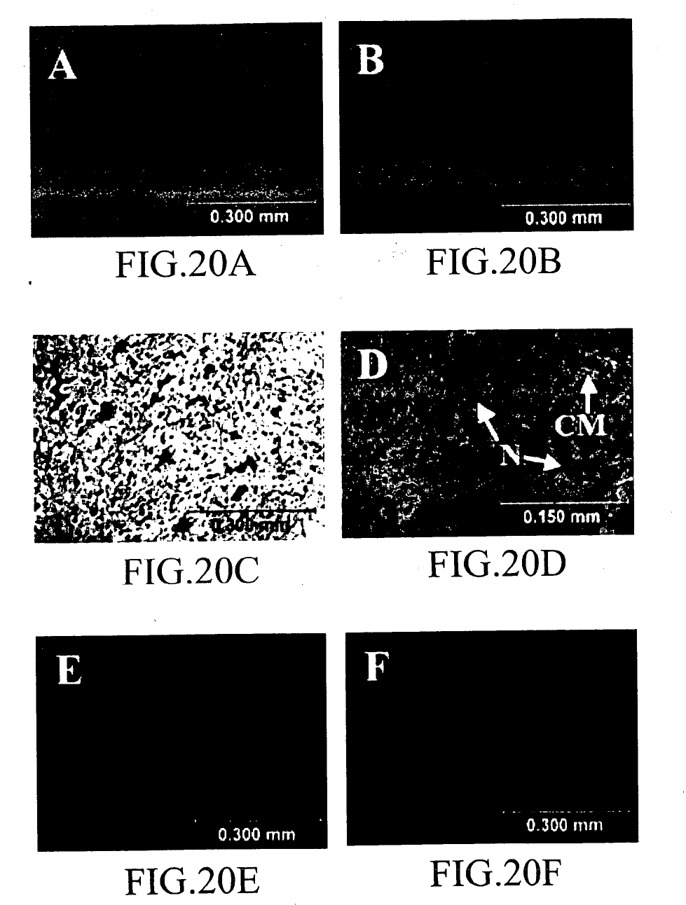

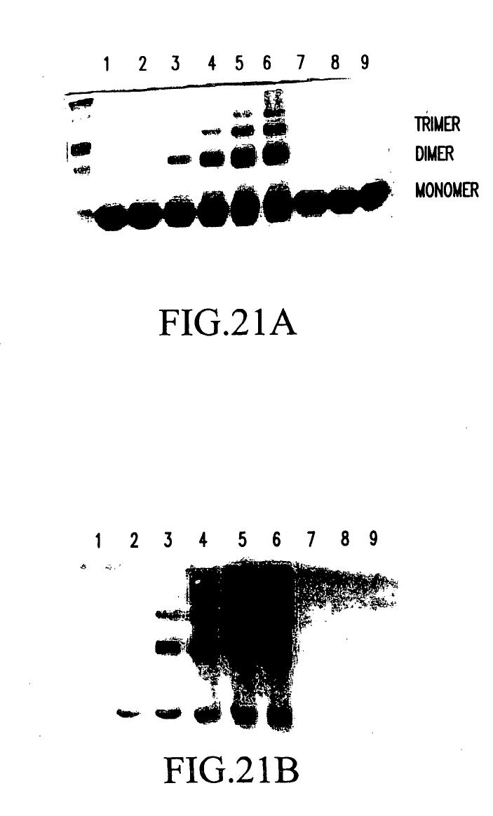

(ii) Results: To explore the spatial heterogeneity of trapping reactions within the liver, immunohistochemical analysis was used to detect hydralazine-stabilized acrolein adducts in sections of right medial liver lobe collected from animals 4 hours after they received allyl alcohol and hydralazine (FIG. 20). Consistent with the results from the Western blot analysis (FIG. 19), the primary antibody did not detect antigens in mice that received injection vehicle only, 90 mg/kg allyl alcohol only or 300 μmol/kg hydralazine only (Panel A of FIG. 20 shows a representative image obtained during analysis of vehicle-treated liver sections—images from allyl alcohol-only and hydralazine only-treated animals were comparable and are omitted due to space considerations). Likewise, no immunorecognition was evident in the livers of mice 4 hours following a large dose of (1E)-acrylaldehyde 1-[1-phthalazinyl]-hydrazone (300 μmol/kg), the main isolable product formed during reactions between free acrolein and hydralazine (Panel B). This confirms that residual tissue levels of this product could not account for any signals detected in allyl alcohol and hydralazine-treated animals.

In striking contrast, intense adduct-trapping was evident in the livers of mice that concurrently received allyl alcohol and 300 μmol/kg hydralazine (Panel C). Analysis of these sections using non-immune rabbit serum instead of primary antibody yielded no signals (i.e. image resembled that shown in Panel A), indicating the strong signals in Panel C were due to specific antigen recognition by the antiserum raised against hydralazine/acrolein-modified KLH. Although it was difficult to identify cellular structures in tissues from allyl alcohol-treated animals that received 300 μmol/kg hydralazine, analysis of liver from a mouse that received 100 μmol/kg hydralazine yielded a clearer image (Panel D). Adduct-trapping reactions are most intense within the cytoplasmic and membrane regions of individual cells (Panel D). Also, while adduct-trapping is evident at nuclear membranes, intranuclear staining is conspicuously absent (Panel D). Since allyl alcohol toxicity is typically localized in periportal regions (1), we expected that immunoreactivity would be most intense in these areas. However, although occasional slices displayed increased staining in periportal sinusoidal cells (data not shown), little zonation of adduct-trapping reactions was evident (Panel D).

Since acrolein can target multiple nucleophilic amino acids during reactions with protein, we assessed the prevalence of adduct-trapping at acrolein-adducted lysine versus histidine residues in liver proteins in vivo. For this experiment, we treated liver sections from allyl alcohol—(90 mg/kg) and hydralazine (100 μmol/kg)-treated mice with primary antibody that had been pre-incubated with either hydralazine/acrolein-modified poly-L-lysine or hydralazine/acrolein-modified poly-L-histidine (Panels E and F respectively). We have previously shown that these modified polyaminoacyl reagents block immunorecognition of hydralazine-stabilized acrolein adducts in a model protein, BSA. Pre-incubation of the primary antibody with 2 mg/ml concentrations of both reagents prior to the immunoassay step strongly inhibited immunorecognition of antigens in liver slices from allyl alcohol and hydralazine-treated animals (Panels E and F).

### EXAMPLE 22

Adduct-trapping by Hydralazine Inhibits Protein Cross-linking by Acrolein

A question that emerges from the “adduct-trapping” action of hydralazine is how this mechanism could account for the strong suppression of acrolein toxicity by the drug. One possibility is that the carbonyl group introduced into proteins by acrolein plays a direct role in the pathogenesis of cell death by acrolein. For example, these adducted proteins might form cross-links with other proteins or DNA, and perhaps this reaction triggers cell death. One possibility is that hydralazine blocks the toxicity of acrolein by trapping these reactive adducted proteins, preventing them from participating in deleterious cross-linking reactions.

To test this possibility, an assay for acrolein-induced protein cross-linking was developed, using bovine pancreas ribonuclease A as a model protein. For this experiment, RNase A (2 mg/mL) was reacted with 0.75, 1.5, 3, 6, or 12 mM acrolein in 50 mM sodium phosphate buffer (pH 7.0). Since concentration of lysine residues in the reaction mixture is 1.5 mM, these concentrations of acrolein represent molar acrolein: lysine ratios of 0, 0.25, 0.5, 1, 2, 4, 8, and 16. After allowing the reaction to proceed for 3 hours at 37° C., the reaction mixtures were resolved by SDS/PAGE on a 14% acrylamide gel. Two gels were run to enable simultaneous assessment of cross-linking and immunochemical detection of acrolein-lysine adducts. Coomassie blue staining was used to analyse the first gel since with this method, monomeric RNase A (non-crosslinked) can be readily distinguished from various cross-linked derivatives that might be generated by acrolein (dimeric RNase A, trimeric RNase A and tetrameric RNase A). The second gel was processed using rabbit antiserum selective for acrolein-modified lysine residues in a Western blotting procedure identical to that described previously in the Patent Application.

The results from this experiment are shown in FIG. 21 below. Lanes 1 to 6 of Panel A indicate that exposing RNase A to increasing concentrations of acrolein for just 3 hours resulted in concentration-dependent formation of cross-linked proteins. Panel B shows that the antibody against acrolein-modified lysine residues detected adducts in acrolein-modified monomeric RNase A and also RNase A dimers, trimers and tetramers. Strikingly, the antibody displayed strongest activity towards cross-linked RNase A, providing an important insight into the epitope for,this antiserum.

FIG. 21 also confirms that adducts at lysine residues are important in the cross-linking reactions, since Lanes 7 to 9 of both Panels of FIG. 21 show that reductively-methylated RNase A was not prone to adduction by acrolein (panel B) or the formation of cross-linked species (Panel A). Reductively-methylated RNase was prepared by dissolving 17 mg RNase A (1.25 μmol) in 490 μL sodium phosphate buffer (0.1 M, pH 7.2), followed by the addition of 125 μmol formaldehyde (10.2 μL of 37% formaldehyde solution) and 125 μmol sodium cyanoborohydride (7.9 mg powder). After allowing reactions to proceed for 18 hours at room temperature, the reaction mixture was dialysed against phosphate buffer using Pierce Slide-A-Lyser 3.5K cut-off dialysis cassettes (0.1 M, pH 7.2) over 24 hours (2 changes of 0.5 L volumes of dialysis buffer). Using a trinitrobenzenesulphonic acid (TNBS)-based assay and isoleucine to prepare a standard curve, it was confirmed that the amine content of the methylated RNase A was <10% of that of native RNase A. The finding that methylated RNase A was not subject to adduction or cross-linking by acrolein shows that lysine residues play a major role in the formation of RNase A aggregates by acrolein.

### EXAMPLE 23

Time-Course of Lysine Adduction and Crosslinking by Acrolein

The above data confirmed that extensive cross-linking of RNase A occurred within a 3 hour reaction period in the presence of acrolein. In addition, earlier data presented here has indicated that interference with steps occurring within 30 to 60 minutes of commencing protein adduction by acrolein might underlie the cytoprotective efficacy of hydralazine.

To establish whether cross-linking might occur over this time-frame, a time-course for RNase A adduction and cross-linking by acrolein was determined. For this experiment, RNase A (2 mg/mL) was reacted with 3 mM acrolein in 50 mM sodium phosphate buffer (pH 7.0). The molar acrolein:lysine ratio was thus 2 in this experiment. Modification was allowed to proceed for up to 3 hours at 37° C. The reaction was performed in a glass vial fitted with a rubber septum, allowing collection of 100 μL aliquots at 30 minute intervals using a needle and syringe (no need to open the vessel at each time point minimised loss of acrolein vapours). At each time point, reaction mixture aliquots were diluted with SDS/PAGE Sample Loading Buffer then stored on ice until the completion of the experiment. The samples were again resolved on two 14% acrylamide gels, with one used for Coomassie Blue staining and the other for immunochemical detection of acrolein-modified lysine adducts.

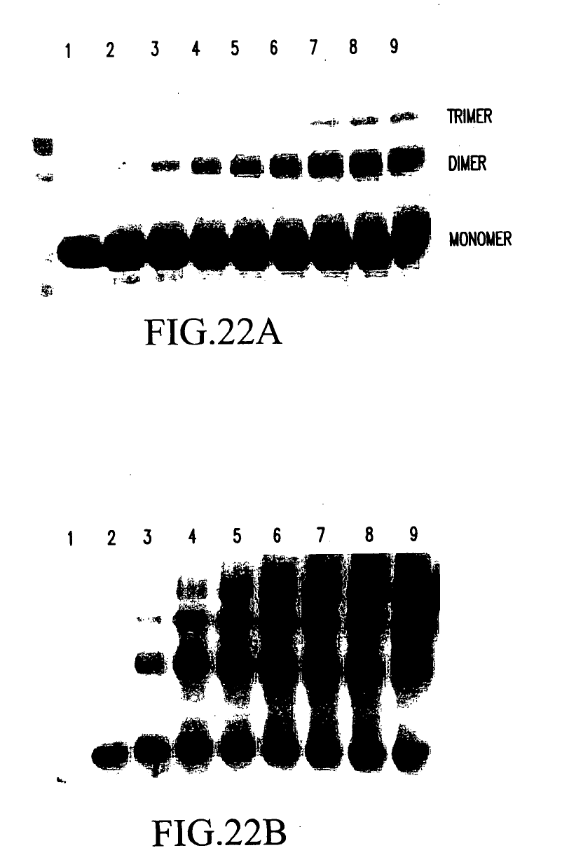

The data shown in FIG. 22 confirms that intermolecular protein cross-linking proceeds at a very fast rate, with dimers evident within just 60 minutes of commencing reactions with acrolein (Lanes 3 and 4 in Panel A of FIG. 22). More importantly, Western blot analysis revealed significant adduction in monomeric RNase A within just 30 minutes of commencing protein modification (Lane 2 of Panel B).This indicates the existence of a time lag between the introduction of acrolein monoadducts on lysine residues and the subsequent generation of cross-linked molecules. One explanation is that hydralazine's cytoprotectiove potency involves an ability to “trap” these adducted proteins during this time-frame, i.e. prior to cross-link formation.

### EXAMPLE 24

Hydralazine Inhibits Cross-linking by Trapping Early Adducts

The following experiment explored the time-dependent susceptibility of acrolein-modified RNase A to hydralazine, to ascertain whether hydralazine traps adducts within the period between initial Michael adduction and subsequent cross-link formation.

For this experiment, RNase A (2.1 mgmL) was treated with 3.2 mM acrolein in 50 mM sodium phosphate buffer (pH 7.0) at 37° C. At 30 and 120 minutes after the commencement of the reaction, aliquots of reaction mixture (190 μL) were diluted with 1, 3 or 9 μL volumes of 60 mM hydralazine, to give final concentrations of 0.3, 1 or 3 mM hydralazine. Appropriate volumes of buffer were added to give a final reaction volume of 200 μL, then the tubes were returned to the incubator for an additional 2 hours. At that time, aliquots of reaction mixture were diluted with SDS/PAGE Sample Loading Buffer then the samples were resolved on three 14% acrylamide gels, with the first used for Coomassie Blue staining (Panel A), and the second and third gels used respectively for the immunodetection of acrolein-lysine adducts (Panel B) or hydralazine-trapped—acrolein adducts (Panel C). The Western blotting conditions were identical for the detection of both acrolein-lysine adducts and hydralazine-trapped adducts.

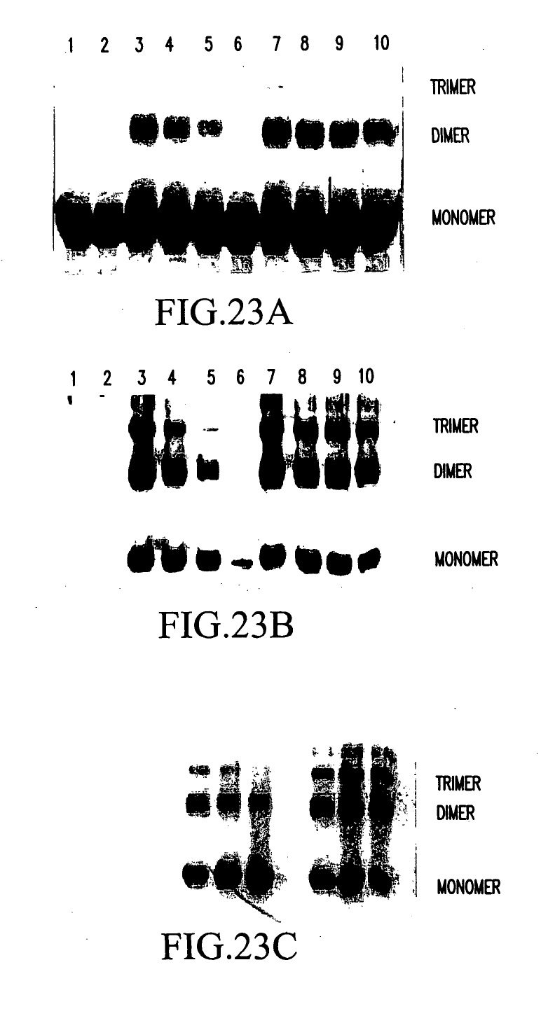

The data is shown in FIG. 23. Adding the drug to final concentrations of 0.3 to 3 mM strongly inhibited formation of crosslinked RNase A, but only if added at the 30 minute time-point (Panel A, Lanes 3 to 6). Thus the drug was incapable of cleaving crosslinked RNase A when it was added 120 minutes after commencing modification by acrolein (Panel A, Lanes 7 to 10). When added at the 30 minute interval, hydralazine abolished the levels of acrolein-lysine adducts (Panel B, Lanes 4 to 6), an effect that was accompanied by strong-adduct trapping, as detected using the antiserum raised against hydralazine/acrolein-modified KLH (Panel C, Lanes 4 to 6). Intriguningly, the drug also participated in trapping-reactions when added at the 120 min time point (Panel C, Lanes 7 to 10). Taken together, these findings confirm that hydralazine blocks cross-linking by trapping “early” intermediates formed during the modification of RNase A by acrolein.

### EXAMPLE 25

Cytoprotection and Adduct Trapping in Neuronal-like Cells

This experiment once again involved PC-12 cells (a cell line obtained from rat renal medullary phaeochromocytomas widely used as model neuron-like cells). In contrast to the experiments previously described here in hepatocytes, allylamine was used instead of allyl alcohol as an acrolein precursor.

PC-12 cells were grown in DMEM (Dulbecco's Modified Eagle's-H21 Medium), which contained 10% horse serum, 5% (v/v) foetal calf serum, 1 mM glutamine, non-essential amino acids, and 10,000 units penicillin/streptomycin in uncoated plastic flasks. Cells were passaged every 3 days. The day prior to an experiment, cell suspensions were re-plated at 4×10⁵ cells/mL in DMEM (100 μL per well) on poly-L-lysine pre-coated 96-well plates. Cells were allowed to attach to plates over night at 37° C. in a 5% CO₂ incubator.

Next day, hydralazine and allylamine were dissolved in phosphate-buffered saline (PBS) and added to culture wells to give respective final concentrations of 100 μM and 0, 20, 40, 60, 80 or 100 μM. In one plate, hydralazine was added along with allylamine, while in a replicate plate the drug was added 4 hours after allylamine (the plate was maintained in the incubator for this time). The plates were then returned to the incubator overnight before the viability of the cells was assessed using the MTT (dimethylthiazol-diphenyltetrazolium bromide) cytotoxicity assay. Briefly, the medium from each well was discarded and replaced with 100 μL MTT solution (0.25 mg/mL MTT dissolved in serum free medium). The plates were returned to the incubator for 2 hrs. The MTT solution was then discarded and 100 μM DMSO was added to each well to lyse the cells and solubilise the formazan product. The formazan was then quantified at 570 nm using a POLARstar Galaxy Microplate reader.

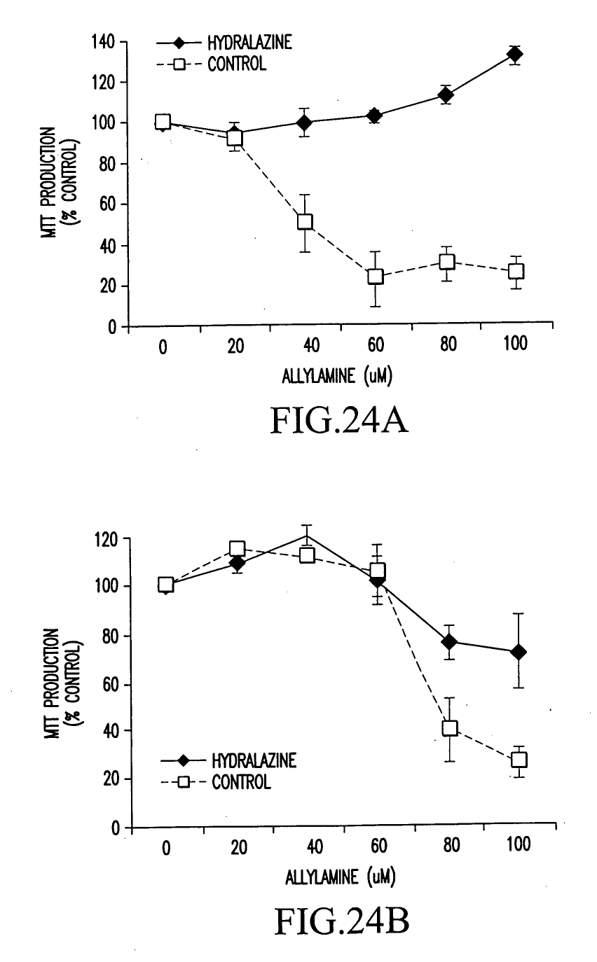

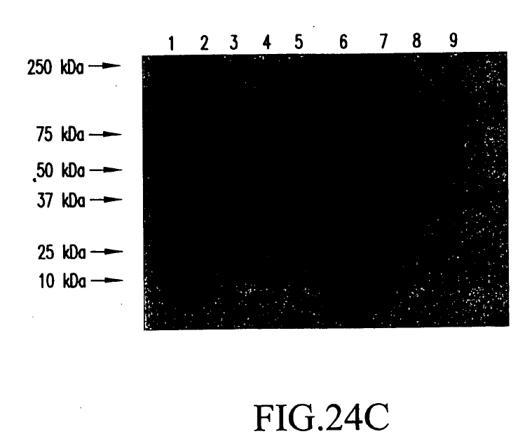

The data is shown in FIG. 24. In Panel A, hydralazine (100 μM) afforded clear protection against the toxicity produced by a range of allylamine concentrations in PC12 cells during an overnight incubation. While the protection was less dramatic, hydralazine also protected when added to cells after a 4 hour prior exposure to the range of allylamine concentrations (Panel B). Most importantly, the cytoprotection was accompanied by intense adduct-trapping reactions in cell proteins, as revealed by the Western blot shown in Panel C. Thus saturating levels of drug-trapped adducts were detected in PC12 cells co-treated with 100 μM hydralazine and either 80 μM allylamine (Panel C, Lanes 6 and 7) or 100 μM allylamine (Lane 2).

### EXAMPLE 26

Cytoprotective Potency Ranking for Selected Hydrazino Compounds Against Allyl Alcohol-Induced Toxicity in Isolated Mouse Hepatocytes

The cytoprotective activity of various hydrazine compounds against allyl-alcohol induced toxicity in isolated mouse hepatocytes was determined essentially as described in Examples 2, 3, 8, 10 and 12.

The PC50 concentration for the various hydrazino compounds producing a 50% decrease in LDH leakage after 60 minutes treatment with 0.1 mM allyl alcohol are shown in Table 2:

TABLE 2
Cytoprotective Potency Ranking for Selected Compounds Against Allyl Alcohol-
Induced Toxicity in Isolated Mouse Hepatocytes
[PC50 = concentration producing a 50% decrease in LDH leakage @ 60 mins, 0.1 mM AA]
(N = 4, mean ± SD)

| | PC₅₀ | | |
|---|---|---|---|
| Compound | (μM) | Structure | Calculated Log P |
| 1,1-diphenylhydrazine | 2.0 ± 0.01 | 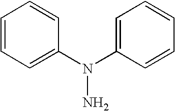 | 2.8 ± 0.2 |
| diphenylamine | 3.0 ± 1.2 | 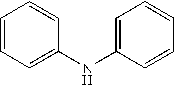 | 2.97 ± 0.287 |
| N,N′-Diphenyl-p- phenylenediamine | 4.0 ± 1.3 | 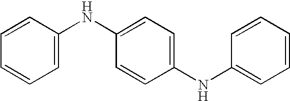 | 3.32 ± 0.413 |
| N-methylbutylamine | 6.0 ± 2.2 | 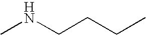 | 1.16 ± 0.2 |
| hydrazinoisoquinoline | 6.0 ± 0.14 | 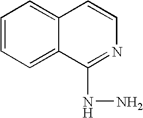 | 1.13 ± 0.3 |
| naphthylhydrazine | 7.0 ± 0.19 | 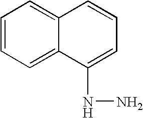 | 2.48 ± 0.28 |
| phenylhydrazine | 8.0 ± 0.28 | 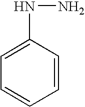 | 1.25 ± 0.28 (experimental) |
| hydrazinoquinazoline | 10 ± 0.05 | 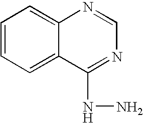 | 1.42 ± 0.4 1.67 (experimental) |
| hydrazinoquinoline | 11 ± 0.44 | 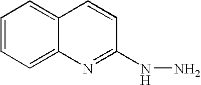 | 1.26 ± 0.31 |
| dihydralazine | 13 ± 0.29 | 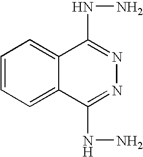 | 0.32 |
| hydralazine | 18 ± 0.81 | 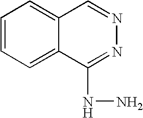 | 1.0 (experimental) |
| o-aminophenol | 18 ± 3.2 | 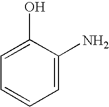 | 0.34 ± 0.21 |
| 1,2-diphenylhydrazine | 19 ± 2.6 | 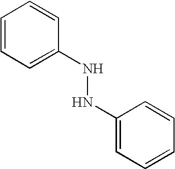 | 2.94 ± 0.29 |
| p-aminophenol | 20 ± 8.5 | 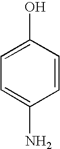 | −0.287 (0.216 experimental) |
| 2,4-dinitro- phenylhydrazine | 21 ± 0.53 | 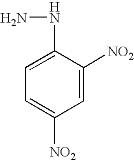 | 1.45 ± 0.33 1.46 (experimental) |
| benzylhydrazine | 37 ± 1.8 | 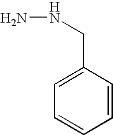 | 0.73 ± 0.21 |
| carbazole | 40 ± 12 | 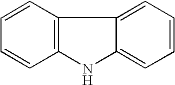 | 3.72 ± 0.252 |
| hydrazinopyridine | 40 ± 6.1 | 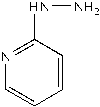 | −0.03 |
| phenol | 48 ± 12 | 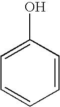 | 1.48 ± 0.185 |
| salicylic acid | 59 ± 11 | 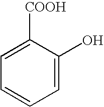 | 2.06 ± 0.247 |
| dicyclohexylamine | 59 ± 11 | 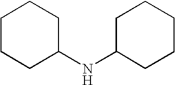 | 3.69 ± 0.206 |
| aniline | 61 ± 20 | 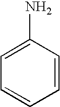 | 0.936 ± 0.189 |
| diisopropylamine | 66 ± 15 | 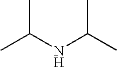 | 1.33 ± 0.212 |
| benzylamine | >100 | 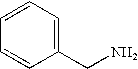 | 1.09 ± 0.209 |
| thiophenol | >100 | 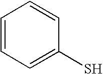 | 2.52 ± 0.277 |
| cyclohexylamine | >100 | 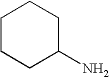 | 1.39 ± 0.187 |
| p-hydroxyamphetamine | >100 | 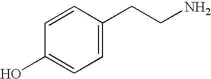 | 0.722 ± 0.207 |
| dimethylhydrazine | >100 | 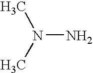 | −1.28 |
| methoxyamine | 9000 ± 1800 | H₃C—O—NH₂ | |
| aminoguanidine | >10,000 | 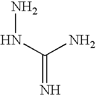 | |
| carnosine | >10,000 | 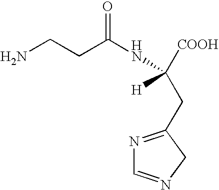 | |
| pyridoxamine | >10,000 | 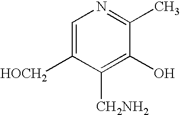 | |

The compounds described above represent specific compounds in the class of compounds with the following chemical formula: 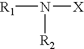
wherein
   X is NH₂ or H;
   R₁ is aryl; substituted aryl including hydrazino-substituted aryl, hydroxy-substituted aryl, and nitro-substituted aryl; heteroaryl; substituted heteroaryl including hydrazino-substituted heteroaryl, hydroxy-substituted heteroaryl, and nitro-substituted heteroaryl; benzyl; anilino; alkylbenzene; C₁ to C₈ alkyl; C₅ to C₈ cycloalkyl; and
   R₂ is aryl; substituted aryl; C₁ to C₈ alkyl; C₅ to C₈ cycloalkyl; or H.

In particular, the hydrazino compounds described above represent specific compounds included in the class of compounds with the following chemical formula:

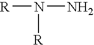

wherein R is H; aryl; substituted aryl including hydrazino-substituted aryl, hydroxy-substituted aryl, and nitro-substituted aryl; heteroaryl; substituted heteroaryl including hydrazino-substituted heteroaryl, hydroxy-substituted heteroaryl, and nitro-substituted heteroaryl; benzyl; anilino; alkylbenzene; C₁ to C₈ alkyl; or C₅ to C₈ cycloalkyl.

### EXAMPLE 27

Aldehyde Sequestering Reactions

Several aldehyde scavengers were compared for their ability to sequester acrolein and crotonaldehyde from buffered solution at physiological temperature (37° C.). As lysine groups are also particularly susceptible to modification by acrolein, the ability of this amino acid to remove acrolein from solution was also examined.

The method used to compare the 2-alkenal scavenging potencies of various nucleophilic reagents was a modification of a previously reported method Burcham et al (2000) Redox Report 5:47-49. Briefly, amine and hydrazine nucleophiles were dissolved in prewarmed sodium phosphate buffer (pH 7.0) to give a final concentration of 1 M before 0.5 ml volumes of these solutions were added to triplicate 1 ml gas chromatography vials. Reactions were started by the addition of prewarmed 0.5 ml volumes of equimolar concentrations of either acrolein or crotonaldehyde (1 M). Vessels were filled to capacity to minimize headspace loss of aldehydes. The vials were then placed in a 37° C. mixing incubator for either 10, 20 or 30 minutes. At each time point, an aliquot was taken from one of the triplicate vials, diluted 1:10 to 1:50 in mobile phase before a 100 μl sample was used for the determination of aldehyde concentrations via HPLC.

The HPLC system comprised an ODS Hypersil column (150×4.6 mm, 5 μm, Keyestone Scientific Inc, PA, USA) connected to a GBC LC1150 pump (Dandenong, Australia), fitted with an online ERC 3415 degasser and a Hewlet Packard series 1100 UV detector that monitored the absorbance of column eluate at 210 nm. The mobile phase used to analyse free acrolein comprised 20% methanol:water (v/v) while in the case of crotonaldehyde, 30% methanol:water (v/v) was used. The flow rate was maintained at 1 ml/minute. Under these conditions, the retention times for acrolein and crotonaldehyde were 2.7 and 3.1 minutes, respectively. Aldehyde concentrations were determined by comparing sample peak areas to those obtained by analysing standard solutions of acrolein and crotonaldehyde (prepared in mobile phase to give final aldehyde concentrations ranging from 0.1 to 12 μM). Output from the UV detector was collected and analysed using Delta Junior HPLC analysis software (Qld, Australia). As the protocol only measured the % loss of free aldehyde, standard curves were not required for analysis of this data.

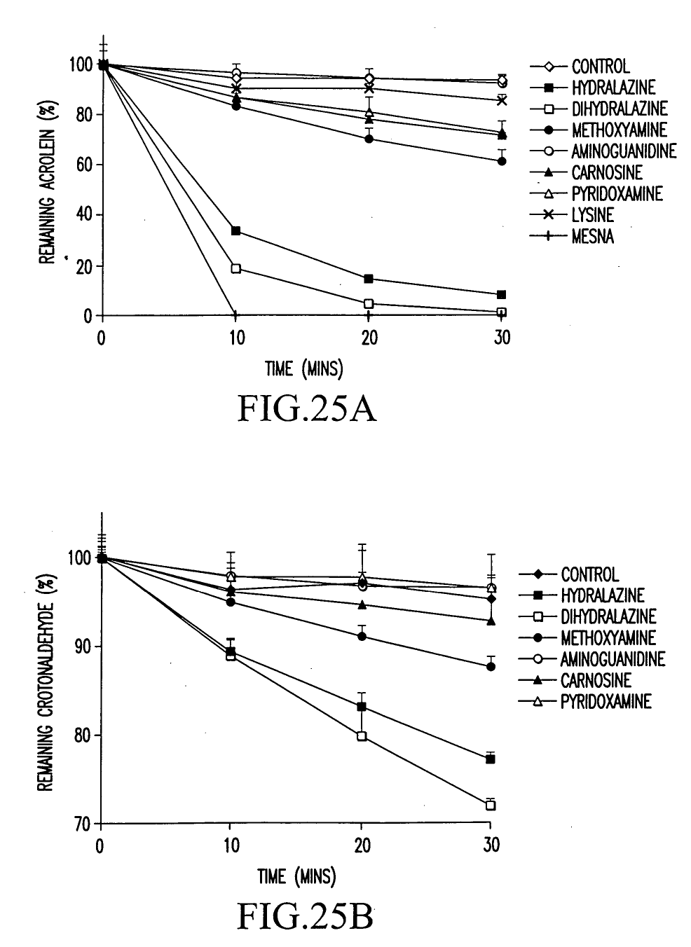

The ability of various aldehyde scavengers to sequester acrolein from solution is shown in FIG. 25. The loss of acrolein due to evaporation into headspace from solution was approximately 4% over 30 minutes. This was similar to the loss of acrolein when co-incubated with an equimolar amount of aminoguanidine, indicating the latter is a very poor acrolein scavenger. The loss of acrolein in the presence of lysine was approximately 13% over 30 minutes followed by carnosine and pyridoxamine which showed similar acrolein scavenging capacity, removing approximately 20% of free acrolein from solution in 30 minutes. Methoxyamine was the most effective amine at sequestering acrolein from solution, having removed around 40% of available acrolein from solution in the same time period. Second to MESNA, hydralazine and dihydralazine were the most effective scavengers of acrolein, with dihydralazine being approximately twice as potent as hydralazine. These compounds removed approximately 82 and 92% of acrolein respectively from solution within 30 minutes of incubation. MESNA removed almost all of the acrolein in solution within 10 minutes of incubation as expected given the high reactivity of the thiol group.

With the exception of N^{α}-acetyl lysine and MESNA, which were not tested against crotonaldehyde, the ability of these compounds to sequester crotonaldehyde from solution resembled their acrolein sequestering ability with the exception that carnosine appeared to be slightly more effective at removing crotonaidehyde from solution than pyridoxamine.

### EXAMPLE 28

Cytoprotection Against Unsaturated Aldehyde-Mediated Toxicity in Isolated Hepatocytes

Lipid peroxidation in vivo results in the production of a variety of structurally diverse α,β-unsaturated aldehydes, including acrolein and crotonaldehyde. α,β-unsaturated aldehydes are also formed as byproducts of the metabolism of a number of clinically used drugs. For instance, the antihypertensive drug Pargyline is associated with hepatotoxicity in humans and rats, via a mechanism involving its biotransformation to the alkynal propiolaldehyde.

To assess whether the 2-alkenal sequestering abilities of hydralazine and dihydralazine confer cytoprotection against the toxicity of acrolein and crotonaldehyde, their ability to protect against the toxicity of the corresponding alcohol precursors was explored in isolated mouse hepatocytes. Alcohol dehydrogenase in mouse hepatocytes readily oxidises allyl and crotyl alcohols to acrolein and crotonaldehyde respectively. In addition to examining the cytoprotection afforded by hydralazine and dihydralazine against these short chained 2-alkenals, the ability of hydralazine to protect against the toxicity of three other unsaturated aldehydes was also examined. These included the prominent enolate malondialdehyde (MDA), pentenal (a longer chained 2-alkenal) and the 2-alkynal propiolaldehyde (which was examined using the precursor, propargyl alcohol). These aldehydes were examined for the purpose of establishing whether hydralazine protects against toxicity mediated wide range of unsaturated aldehydes.

Hepatocytes were isolated via collagenase digestion of the livers of anaesthetised mice using a previously described method (Harman et al, 1987). After filtering suspensions through 200 and 100 μm nylon gauze, cells were washed via three rounds of centrifugation and resuspension in Krebs-Henseleit buffer (supplemented with 1 mM CaCl₂). Finally, cells were suspended in RPMI medium (supplemented with 0.03% L-glutamine, 0.2% bovine serum albumin and penicillin/streptomycin (50 units/l and 50 μg/ml respectively) at a density of 1×10⁶ cells/ml and were plated on collagen-coated dishes (60 mm diameter, IWAKI, Japan, 3 ml cell suspension per plate). Cells were allowed to attach to the dishes for 2 to 3 h in a humidified atmosphere of 5% CO₂ and 95% air at 37° C. before use.

Prior to initial experimentation, brief concentration responses for the cytotoxicity of propargyl alcohol, pentenal and MDA were performed on concentrations of toxin ranging from 100 μM to 10 mM for up to 24 hours (data not shown). For propargyl alcohol and pentenal, concentrations were chosen that resulted in maximal or near maximal cell death within 3 hours. MDA did not induce toxicity until 12 hours after the addition of the aldehyde at 10 mM and was therefore assessed at a later time point than for the other aldehydes.

Plated cells were washed with PBS (50 mM, pH 7.4; 3 ml per plate for 60 mm dishes) to remove nonadherent cells before they were incubated with either culture media alone (supplemented with L-glutamine and penicillin/streptomycin as above) or supplemented with one of the scavengers (1-100 μM; hydralazine, dihydralazine) for 5 minutes prior to the addition of allyl alcohol (100 μM), crotyl alcohol (500 μM), pentenal (1 mM), propargyl alcohol (1 mM) or MDA (10 mM) (cytoprotection by dihydralazine was only examined for allyl and crotyl alcohols). Nucleophilic compounds and unsaturated alcohols were dissolved directly in culture media. Dishes were then returned to the incubator for 3 hours (18 hours for MDA) with aliquots of culture media taken at 1 hourly intervals (2 hourly intervals starting from 12 hours after addition of MDA to the culture media) to assess lactate dehydrogenase (LDH) leakage from the cytoplasm. LDH activity in culture media was assayed using a modified method to allow concurrent determination of multiple samples using a fluorescence microplate reader (POLARStar, BMG Laboratories). The reaction followed NADH formation from NAD⁺ in tris buffer (50 mM, pH 8) in the presence of 7 mM NAD⁺ and 25 mM lactic acid at respective excitation and emission wavelengths of 320 and 460 nm. Cell death was reported as % LDH leakage compared to total cellular LDH, which was determined by sonicating the cells with 0.5% Triton® X-100 (1/10 dilution of 5% Tritone X-100 in culture medium containing cells).

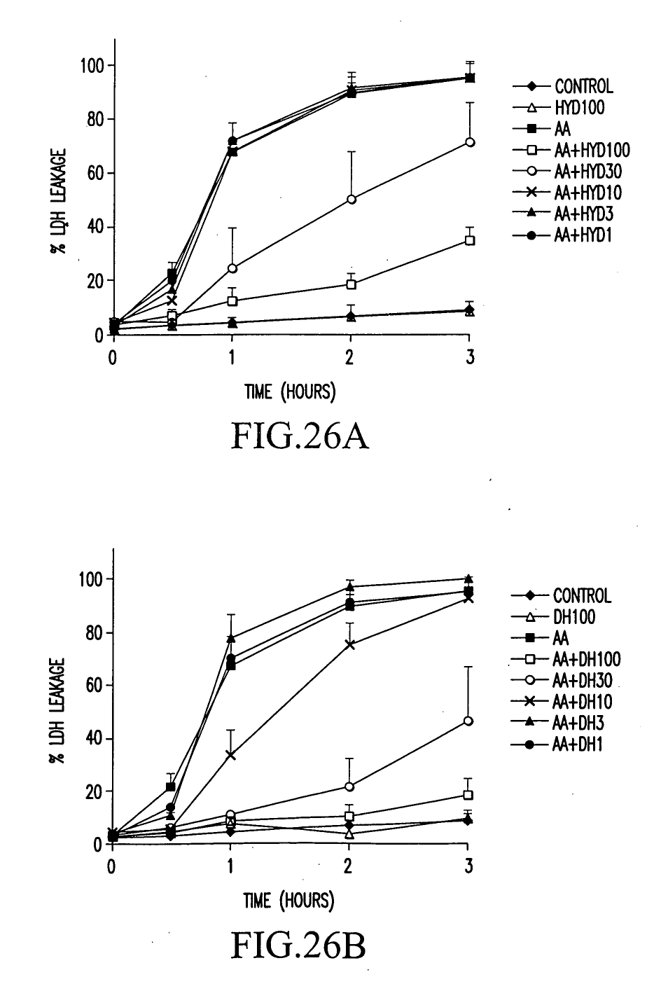

FIG. 26 shows the concentration dependent protection of allyl alcohol toxicity by hydralazine and dihydralazine respectively over 3 hours. Allyl alcohol induced 100% cell death within 3 hours of its addition to hepatocytes. Hydralazine (Panel A) and dihydralazine (Panel B) both inhibited this toxicity in a concentration dependent manner, with dihydralazine approximately twice as protective as hydralazine.

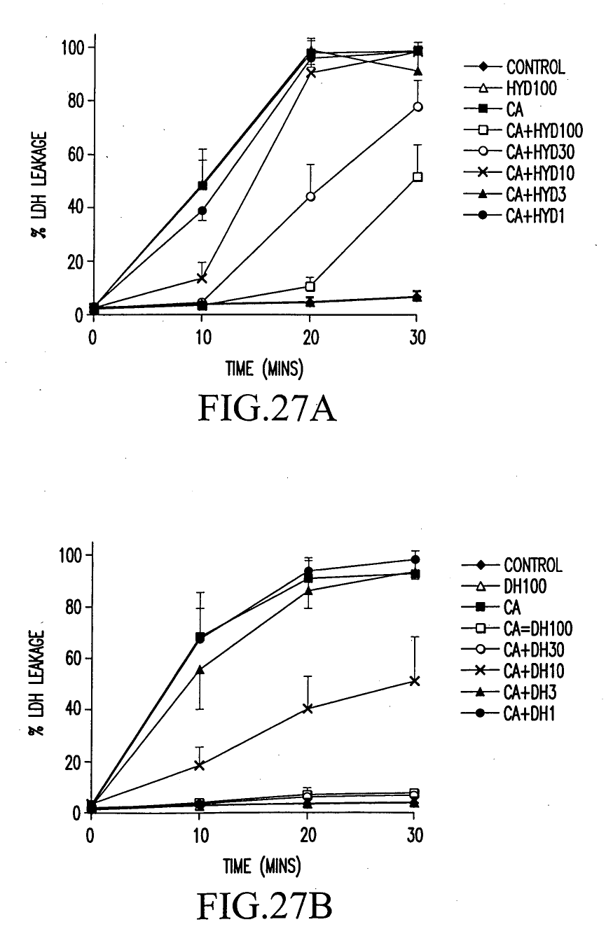

Crotyl alcohol similarly induced 100% cell death by 3 hours of incubation, as shown in FIG. 27. A similar concentration dependence in hydralazine (Panel A) and dihydralazine (Panel B) protection was seen where dihydralazine was approximately twice as effective as hydralazine at inhibiting this toxicity. This showed that hydralazine and dihydralazine protect against toxicity mediated by two different short chained 2-alkenals.

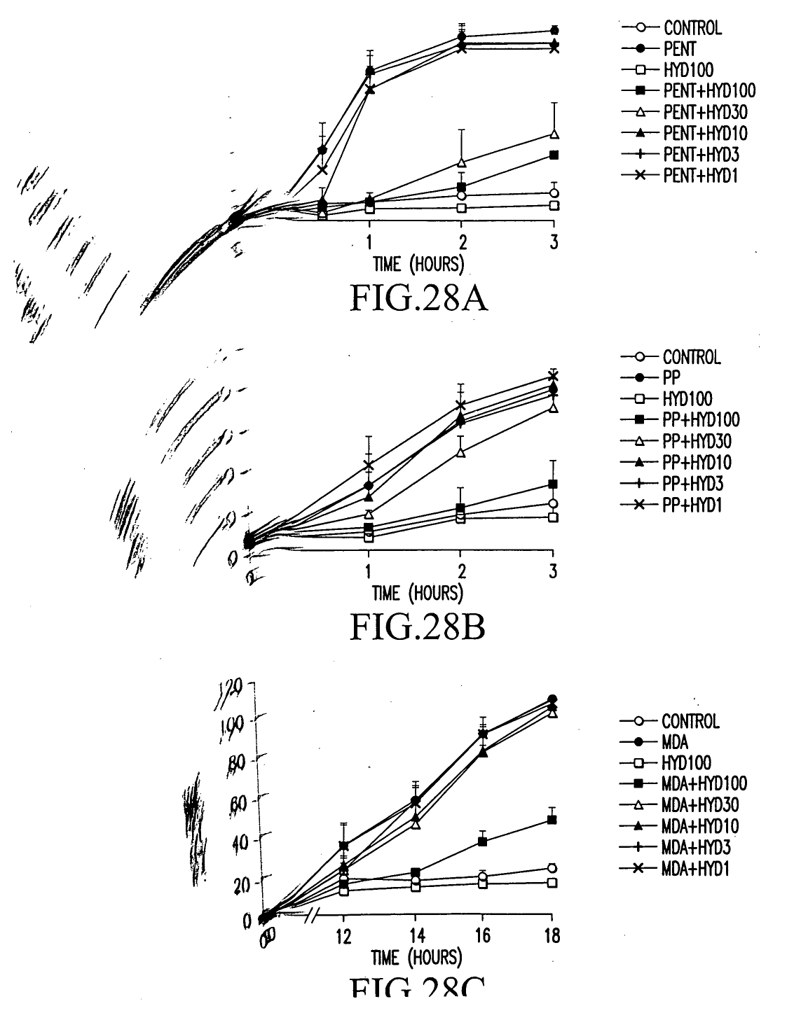

Pentenal and propargyl alcohol both induced maximal cell death by 3 hours of incubation as shown in FIG. 28. MDA had a much slower onset of toxicity, taking 12 hours before noticeable toxicity began (Panel A, FIG. 28). Hydralazine protected against cell death induced by all the aldehydes or their precursors in a concentration dependent manner, although the protection was most efficient for pentenal toxicity. Complete protection against propargyl alcohol and MDA toxicity was only achieved at the highest concentration of hydralazine used (100 μM). Nevertheless, the data indicated that hydralazine protects against a range of α,β-unsaturated aldehydes.

### EXAMPLE 29

Acrolein Scavenging Capacity of Other Hydrazines and Hydralazine Analogues

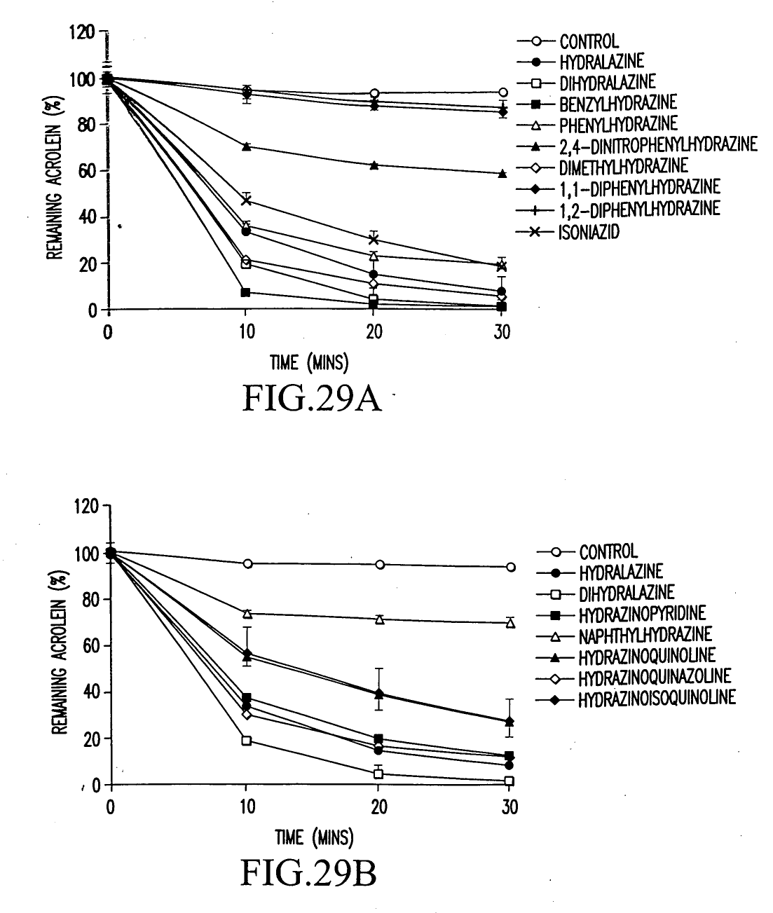

While the reactivity of most amine nucleophiles with 2-alkenals is due to the nucleophilicity of the reacting amine, the possibility that the greater reactivity of hydralazine and dihydralazine is due to the presence of their hydrazine groups was examined. This was achieved by screening a number of structurally diverse hydrazines and phthalazine analogues for their ability to sequester acrolein from solution, as it was expected that the hydrazine functional groups on each of these compounds would confer better 2-alkenal sequestering effects than aminoguanidine, pyridoxamine, methoxyamine and carnosine. The acrolein sequestering effects of the hydrazines (Panel A, FIG. 29) and phthalazine analogues (Panel B, FIG. 29) were compared to hydralazine and dihydralazine.

Solutions of acrolein were made in phosphate buffer (50 mM). Stock solutions (10 mM) of 2-hydrazinoquinoline, 1-hydrazinoquinazoline, 2-hydrazinopyridine, naphthylhydrazine, 1,1-diphenylhydrazine, 1,2-diphenylhydrazine, dimethylhydrazine, isoniazid and 2,4-dinitrophenylhydrazine were made in acetonitrile/buffer mixtures depending on solubility. Phenylhydrazine and benzylhydrazine were diluted in various methanol/buffer mixtures depending on solubility. 1-Hydrazinoisoquinoline was diluted in DMSO. The proportion of solvent in the final reaction mixture was therefore ≦5%. It was also determined that the presence of 5% of these solvents in the final reaction mixture did not alter the ability of hydralazine to sequester acrolein in solution. The ability of these compounds to scavenge acrolein from buffered solution was then determined.

As shown in FIG. 29 in Panel A, the most effective hydrazine examined for its acrolein scavenging ability was benzylhydrazine (BH), which removed almost all acrolein from solution within 20 minutes of incubation. In descending order of potency the next most efficient hydrazines were dihydralazine (DH), 1,1-dimethylhydrazine (DMH), hydralazine (HYD), phenylhydrazine (PH), isoniazid, 1,1-diphenylhydrazine (1,1-DPH), 1,2-diphenylhydrazine (1,2-DPH). 2,4-Dinitrophenylhydrazine was only able to remove around 40% of acrolein from solution after 30 minutes of incubation, while 1,2-diphenylhydrazine was the least effective, having removed only around 10% of acrolein from solution in this time period.

The hydralazine analogues naphthylhydrazine (NH), 2-hydrazinopyridine (HP), 4-hydrazinoquinazoline (HQZ), 2-hydrazinoquinoline (HQL) and 1-hydrazinoisoquinoline (HIQ) were examined for acrolein scavenging capacity, with the results shown in FIG. 29, Panel B.

Naphthylhydrazine (NH) was the poorest scavenger of the phthalazine analogues, having removed 28% of free acrolein from solution in 10 minutes. 2-Hydrazinoquinoline (HQL) and 1-hydrazinoisoquinoline (HIQ) were the next most effective analogues having identical acrolein scavenging profiles. These compounds removed approximately 70% of free acrolein from solution in 30 minutes. 4-Hydrazinoquinazoline (HQZ), 2-hydrazinopyridine (HP) and hydralazine (HYD) were the next most effective scavengers with 2-hydrazinopyridine (HP) appearing to be slightly less effective than the other 2 compounds. These compounds sequestered approximately 85 to 90% of acrolein in solution in 30 minutes. Dihydralazine (DH) was again the most effective scavenger among the hydrazinophthalazines.

### EXAMPLE 30

Cytoprotection Crotyl Alcohol Toxicity by Other Hydrazines and Hydralazine Analogues

The hydrazines 1-hydrazinoisoquinoline (HIQ), 2-hydrazinoquinoline (HQL), 4-hydrazinoquinazoline (HQZ), 1,1-diphenylhydrazine (1,1-DPH) and benzylhydrazine (BH) were also compared for their ability to protect against crotyl alcohol toxicity in mouse hepatocytes. Given that crotyl alcohol does not mediate short term toxicity in cells cultured in 96 well plates, larger dishes (60 mm diameter) were used for these experiments.

Cells were treated with 500 μM crotyl alcohol and 1-100 μM of the hydrazines or amines as previously described in a final volume of 100 μl RPMI. All nucleophilic compounds were prepared in stock solutions of 50 mM in DMSO prior to dilution in culture media. The hydrazines hydrazinoisoquinoline, hydrazinoquinoline, hydrazinoquinazoline, 1,1-diphenylhydrazine and benzylhydrazine were compared for their ability to similarly protect against crotyl alcohol toxicity in 60 mm dishes as previously reported for hydralazine and dihydralazine. Aliquots of media (10 μl) were taken 1 and 2 hours after the addition of allyl alcohol and 1, 2 and 3 hours after crotyl alcohol and assayed for LDH activity. Cell death was measured as the % LDH leakage from the cells into the media compared to total cellular LDH as described previously. Total cellular LDH was measured by sonicating each well after the addition of 10 μl PBS and 10 μl 5% Triton® X-100 (for allyl alcohol) or 300 μl 5% Triton® X-100 (for crotyl alcohol) to give a final Triton® concentration of 0.5%.

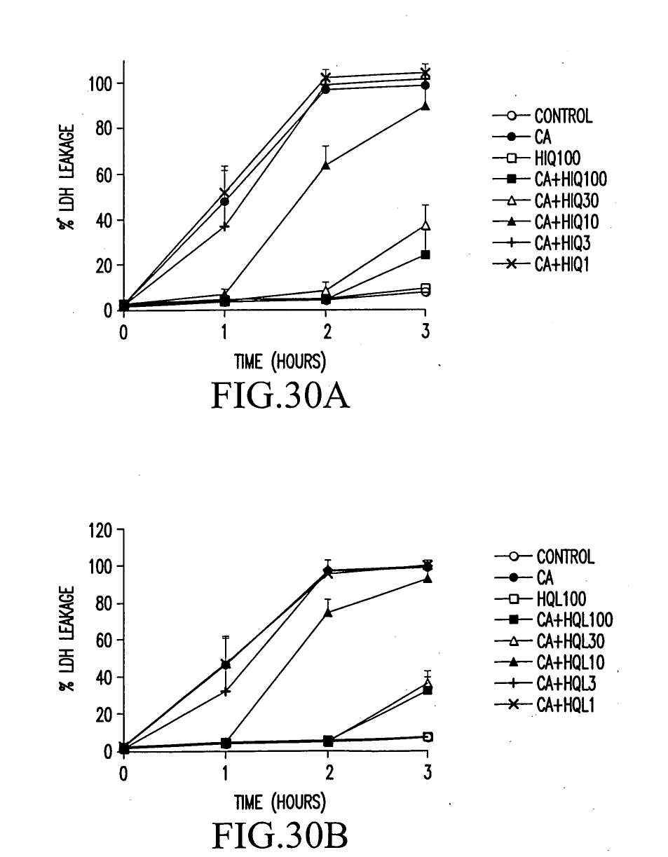

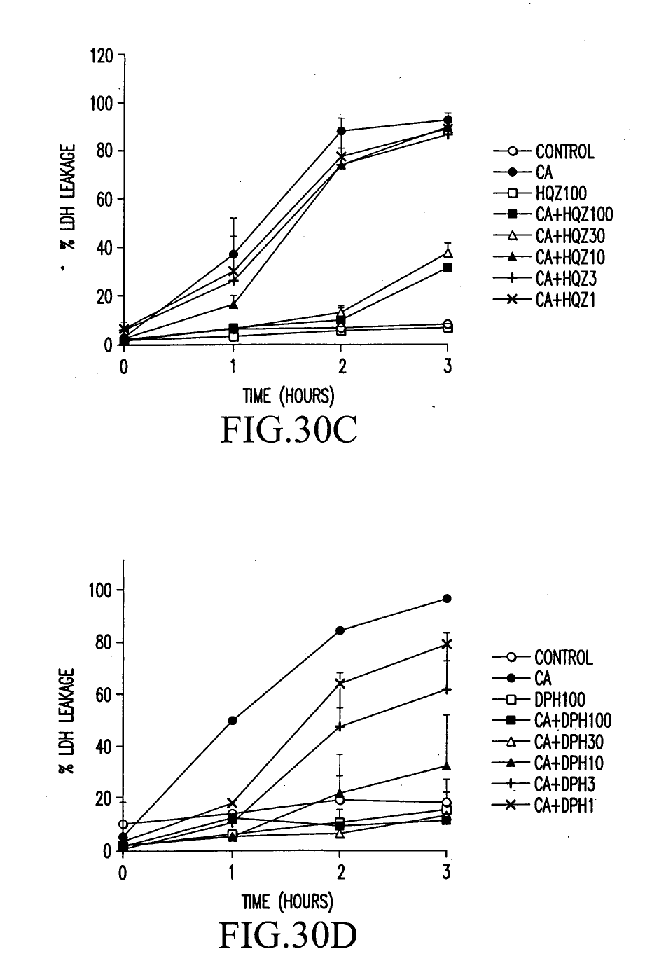

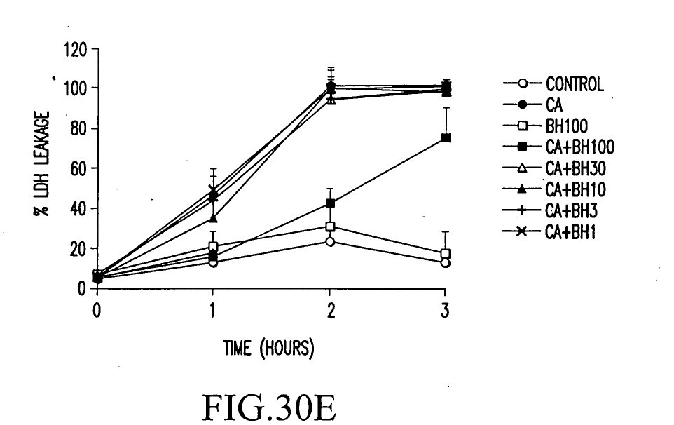

As shown in FIG. 30, the ability of these hydrazines to protect against crotyl alcohol toxicity closely resembled their efficacy against allyl alcohol toxicity (Table 2), where 1,1-diphenylhydrazine provided the best protection while benzylhydrazine afforded very poor cytoprotection.

Finally, it will be appreciated that there may be other variations and modifications to the methods described herein that are also within the scope of the present invention.

1. A method of preventing and/or treating a disease or condition associated with damage mediated by an α,β-unsaturated aldehyde in a subject, the method including the step of administering to the subject a therapeutically effective amount of hydralazine and/or dihydralazine.

2. A method according to claim 1, wherein the α,β-unsaturated aldehyde is acrolein, malondialdehyde, a 4-hydroxyalkenal, a dienal, a 2-alkenal, or the reactive α,β-unsaturated aldehyde tautomers of any of these compounds.

3. A method according to claim 1, wherein the disease or condition is a neurodegenerative disease or condition.

4. A method according to claim 1, wherein the condition is associated with cyclophosphamide chemotherapy.

5. A method according to claim 1, wherein the condition is acute or chronic exposure to smoke.

6. A method of determining the extent of damage mediated by an α,β-unsaturated aldehyde in a biological system, the method including the step of determining the concentration of one or more α,β-unsaturated aldehyde-modified proteins in the biological system.

7. A method according to claim 6, wherein the α,β-unsaturated aldehyde is acrolein, malondialdehyde, a 4-hydroxyalkenal, a dienal, a 2-alkenal, or the reactive α,β-unsaturated aldehyde tautomers of any of these compounds.

8. A method according to claim 6, wherein the biological system is an animal or human.

9. A method according to claim 6, wherein the determination of the concentration of the one or more α,β-unsaturated aldehyde-modified proteins includes the use of an antibody raised to an α,β-unsaturated aldehyde-modified protein to detect the one or more α,β-unsaturated aldehyde-modified proteins.

10. A method of identifying a molecule that reduces the concentration of an acrolein-modified protein in a cell, the method including the steps of:
(a) exposing the cell to a test molecule;
(b) determining the ability of the test molecule to reduce the concentration of an acrolein-modified protein in the cell; and
(c) identifying the test molecule as a molecule capable of reducing the concentration of an acrolein-modified protein in the cell.

11. A method according to claim 10, wherein the acrolein-modified protein is formed by the reaction of a protein with endogenously produced acrolein.

12. A method according to claim 10, wherein the acrolein-modified protein is formed by the exposure of the cell to exogenous acrolein or an acrolein precursor.

13. A method according to claim 10, wherein the determination of the concentration of the acrolein-modified protein includes the use of an antibody raised to an acrolein-modified protein to detect the acrolein-modified protein.

14. A molecule identified according to the method of claim 10.

15. A method of preventing and/or treating a disease or condition associated with damage mediated by an α,β-unsaturated aldehyde in a subject, the method including the step of administering to the subject a therapeutically effective amount of a hydrazino compound.

16. A method according to claim 15, wherein the hydrazino compound is a compound with the following chemical formula: 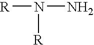
or a pharmaceutically acceptable salt thereof; wherein R is H; aryl; substituted aryl including hydrazino-substituted aryl, hydroxy-substituted aryl, and nitro-substituted aryl; heteroaryl; substituted heteroaryl including hydrazino-substituted heteroaryl, hydroxy-substituted heteroaryl, and nitro-substituted heteroaryl; benzyl; anilino; alkylbenzene; C₁ to C₈ alkyl; or C₅ to C₈ cycloalkyl.

17. A method according to claim 16, wherein the hydrazino compound is selected from the group consisting of 1,1-diphenylhydrazine, hydrazinoisoquinoline, naphthylhydrazine, phenylhydrazine, hydrazinoquinazoline, hydrazinoquinoline, dihydralazine, hydralazine, 1,2-diphenylhydrazine, 2,4-dinitro-phenylhydrazine, benzylhydrazine, hydrazinopyridine, dimethylhydrazine, and aminoguanidine.

18. A method according to claim 15, wherein the α,β-unsaturated aldehyde is acrolein, malondialdehyde, a 4-hydroxyalkenal, a dienal, a 2-alkenal, or the reactive α,β-unsaturated aldehyde tautomers of any of these compounds.

19. A method according to claim 15, wherein the disease or condition is a neurodegenerative disease or condition.

20. A method according to claim 15, wherein the condition is associated with cyclophosphamide chemotherapy.

21. A method according to claim 15, wherein the condition is acute or chronic exposure to smoke.

22. A method of inhibiting cross-linking of molecules by an α,β-unsaturated aldehyde, the method including the step of inhibiting formation of an adduct of a first molecule with an α,β-unsaturated aldehyde and/or inhibiting reaction of the adduct with a second molecule to cross-link the molecules.

23. A method according to claim 22, wherein the α,β-unsaturated aldehyde is acrolein, malondialdehyde, a 4-hydroxyalkenal, a dienal, a 2-alkenal, or the reactive α,β-unsaturated aldehyde tautomers of any of these compounds.

24. A method according to claim 22, wherein the inhibition of reaction of the adduct with the second molecule to cross-link the first and second molecules involves inhibition of reaction of a carbonyl group on the adduct with a reactive group on the second molecule.

25. A method according to claim 22, wherein the first molecule is a protein.

26. A method according to claim 22, wherein the second molecule is a protein or a nucleic acid.

27. A method according to claim 22, wherein the inhibition of cross-linking includes exposure of the first molecule to an agent that inhibits adduct formation and/or inhibits reaction of the adduct with the second molecule.

28. A method according to claim 27, wherein the agent reacts with a carbonyl group on the adduct to inhibit the carbonyl group reacting with a reactive group on the second molecule.

29. A method according to claim 27, wherein the agent is a hydrazino compound.

30. A method according to claim 29, wherein the hydrazino compound is a compound with the following chemical formula: 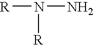
or a salt thereof; wherein R is H; aryl; substituted aryl including hydrazino-substituted aryl, hydroxy-substituted aryl, and nitro-substituted aryl; heteroaryl; substituted heteroaryl including hydrazino-substituted heteroaryl, hydroxy-substituted heteroaryl, and nitro-substituted heteroaryl; benzyl; anilino; alkylbenzene; C₁ to C₈ alkyl; or C₅ to C₈ cycloalkyl.

31. A method according to claim 30, wherein the hydrazino compound is selected from the group consisting of 1,1-diphenylhydrazine, hydrazinoisoquinoline, naphthylhydrazine, phenylhydrazine, hydrazinoquinazoline, hydrazinoquinoline, dihydralazine, hydralazine, 1,2-diphenylhydrazine, 2,4-dinitro-phenylhydrazine, benzylhydrazine, hydrazinopyridine, dimethylhydrazine, and aminoguanidine.

32. A method according to claim 22, wherein the inhibition of cross-linking of molecules occurs in a biological system.

33. A method according to claim 32, wherein the biological system is an animal or human.

34. A method according to claim 33, wherein the human is susceptible to, or suffering from, a neurodegenerative disease or condition.

35. A method according to claim 33, wherein the human is susceptible to, is undergoing, or has undergone cyclophosphamide chemotherapy.

36. A method according to claim 33, wherein the human is susceptibel to, or suffering from, acute or chronic exposure to smoke.

37. A method of reducing damage mediated by an α,β-unsaturated aldehyde in a biological system, the method including the step of administering to the biological system an effective amount of an agent that inhibits cross-linking of molecules by the α,β-unsaturated aldehyde in the biological system.

38. A method according to claim 37, wherein the α,β-unsaturated aldehyde is acrolein, malondialdehyde, a 4-hydroxyalkenal, a dienal, a 2-alkenal, or the reactive α,β-unsaturated aldehyde tautomers of any of these compounds.

39. A method according to claim 37, wherein the agent inhibits cross-linking of proteins or inhibits cross-linking of a protein to a nucleic acid.

40. A method according to claim 37, wherein the agent inhibits cross-linking by inhibiting formation of an adduct of a first molecule with an α,β-unsaturated aldehyde and/or by inhibiting reaction of the adduct with a second molecule to cross-link the molecules.

41. A method according to claim 40, wherein the agent inhibits reaction of a carbonyl group on the adduct with a reactive group on the second molecule.

42. A method according to claim 37, wherein the agent is a hydrazino compound.

43. A method according to claim 42, wherein the hydrazino compound is a compound with the following chemical formula: 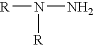
or a salt thereof; wherein R is H; aryl; substituted aryl including hydrazino-substituted aryl, hydroxy-substituted aryl, and nitro-substituted aryl; heteroaryl; substituted heteroaryl including hydrazino-substituted heteroaryl, hydroxy-substituted heteroaryl, and nitro-substituted heteroaryl; benzyl; anilino; alkylbenzene; C₁ to C₈ alkyl; or C₅ to C₈ cycloalkyl.

44. A method according to claim 43, wherein the hydrazino compound is selected from the group consisting of 1,1-diphenylhydrazine, hydrazinoisoquinoline, naphthylhydrazine, phenylhydrazine, hydrazinoquinazoline, hydrazinoquinoline, dihydralazine, hydralazine, 1,2-diphenylhydrazine, 2,4-dinitro-phenylhydrazine, benzylhydrazine, hydrazinopyridine, dimethylhydrazine, and aminoguanidine.

45. A method according to claim 37, wherein the biological system is an animal or human.

46. A method according to claim 37, wherein the damage is due to endogenous production of the α,β-unsaturated aldehyde in the biological system.

47. A method according to claim 37, wherein the damage is due to exposure of the biological system to exogenous α,β-unsaturated aldehyde or an α,β-unsaturated aldehyde precursor.

48. A method according to claim 37, wherein the damage is due to cyclophosphamide chemotherapy.

49. A method according to claim 37, wherein the damage is due to acute or chronic exposure to smoke.

50. A method of preventing and/or treating a disease or condition associated with damage mediated by an α,β-unsaturated aldehyde in a subject, the method including the step of administering to the subject a therapeutically effective amount of an agent that inhibits cross-linking of molecules by the α,β-unsaturated aldehyde.

51. A method according to claim 50, wherein the α,β-unsaturated aldehyde is acrolein, malondialdehyde, a 4-hydroxyalkenal, a dienal, a 2-alkenal, or the reactive α,β-unsaturated aldehyde tautomers of any of these compounds.

52. A method according to claim 50, wherein the agent inhibits cross-linking of proteins or inhibits cross-linking of a protein to a nucleic acid.

53. A method according to claim 50, wherein the agent inhibits cross-linking by inhibiting formation of an adduct of a first molecule with an α,β-unsaturated aldehyde and/or by inhibiting reaction of the adduct with a second molecule to cross-link the molecules.

54. A method according to claim 53, wherein the agents inhibits reaction of a carbonyl group on the adduct with a reactive group on the second molecule.

55. A method according to claim 50, wherein the agent is a hydrazino compound.

56. A method according to claim 55, wherein the hydrazino compound is a compound with the following chemical formula: 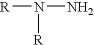
or a salt thereof; wherein R is H; aryl; substituted aryl including hydrazino-substituted aryl, hydroxy-substituted aryl, and nitro-substituted aryl; heteroaryl; substituted heteroaryl including hydrazino-substituted heteroaryl, hydroxy-substituted heteroaryl, and nitro-substituted heteroaryl; benzyl; anilino; alkylbenzene; C₁ to C₈ alkyl; or C₅ to C₈ cycloalkyl.

57. A method according to claim 56, wherein the hydrazino compound is selected from the group consisting of 1,1-diphenylhydrazine, hydrazinoisoquinoline, naphthylhydrazine, phenylhydrazine, hydrazinoquinazoline, hydrazinoquinoline, dihydralazine, hydralazine, 1,2-diphenylhydrazine, 2,4-dinitro-phenylhydrazine, benzylhydrazine, hydrazinopyridine, dimethylhydrazine, and aminoguanidine.

58. A method according to claim 50, wherein the disease is a neurodegenerative disease.

59. A method according to claim 50, wherein the condition is associated with cyclophosphamide chemotherapy.

60. A method according to claim 50, wherein the condition is acute or chronic exposure to smoke.

61. A method of determining the extent of damage mediated by an α,β-unsaturated aldehyde in a biological system, the method including the step of determining the concentration of one or more cross-linked molecules in the biological system.

62. A method according to claim 61, wherein the α,β-unsaturated aldehyde is acrolein, malondialdehyde, a 4-hydroxyalkenal, a dienal, a 2-alkenal, or the reactive α,β-unsaturated aldehyde tautomers of any of these compounds.

63. A method according to claim 61, wherein the determination of the concentration of the one or more cross-linked molecules includes the use of an antibody to detect the cross-linked molecules

64. A method of identifying a molecule that inhibits cross-linking of molecules by an α,β-unsaturated aldehyde, the method including the steps of:
(a) exposing a substrate to an α,β-unsaturated aldehyde;
(b) determining the ability of a test molecule to inhibit cross-linking of the substrate by the α,β-unsaturated aldehyde to another molecule; and
(c) identifying the test molecule as a molecule that inhibits cross-linking of molecules by an α,β-unsaturated aldehyde by the ability of the test molecule to inhibit cross-linking of the substrate.

65. A method according to claim 64, wherein the α,β-unsaturated aldehyde is acrolein, malondialdehyde, a 4-hydroxyalkenal, a dienal, a 2-alkenal, or the reactive α,β-unsaturated aldehyde tautomers of any of these compounds.

66. A method according to claim 64, wherein the substrate is a protein.

67. A method according to claim 66, wherein the protein is cross-linked to another protein or cross-linked to a nucleic acid.

68. A method according to claim 64, wherein the inhibition of cross-linking of the substrate occurs in a cell.

69. A molecule identified according to the method of claim 64.

70. An antibody, or an antigen binding portion thereof, that binds to an α,β-unsaturated aldehyde-hydrazino compound adduct.
